SEQUENCE LISTING (25 sequences)

SEQ ID NO: 1 - DNA, 20 bp, Artificial Sequence
  Description of Artificial Sequence Synthetic primer
gagcaagggcgaggagctgt

SEQ ID NO: 2 - DNA, 24 bp, Artificial Sequence
  Description of Artificial Sequence Synthetic primer
tcttcttcaaggacgacggcaact

SEQ ID NO: 3 - DNA, 20 bp, Artificial Sequence
  Description of Artificial Sequence Synthetic primer
tgctgcccgacaaccactac

SEQ ID NO: 4 - DNA, 18 bp, Artificial Sequence
  Description of Artificial Sequence Synthetic primer
gagggcgagggcgatgcc

SEQ ID NO: 5 - DNA, 20 bp, Artificial Sequence
  Description of Artificial Sequence Synthetic primer
gcttgccctgtccagttaat

SEQ ID NO: 6 - DNA, 20 bp, Artificial Sequence
  Description of Artificial Sequence Synthetic primer
ccgtaggtcagggtggtcac

SEQ ID NO: 7 - DNA, 24 bp, Artificial Sequence
  Description of Artificial Sequence Synthetic primer
ttgtagttgtactccagcttgtgc

SEQ ID NO: 8 - DNA, 20 bp, Artificial Sequence
  Description of Artificial Sequence Synthetic primer
gtacagctcgtccatgccga

SEQ ID NO: 9 - DNA, 18 bp, Artificial Sequence
  Description of Artificial Sequence Synthetic primer
gccttcgggcatggcgga

SEQ ID NO: 10 - DNA, 20 bp, Artificial Sequence
  Description of Artificial Sequence Synthetic primer
tagctcagctgcacccttta

SEQ ID NO: 11 - DNA, 18 bp, Artificial Sequence
  Description of Artificial Sequence Synthetic incorporated RNA substrate sequence
  Description of Combined DNA/RNA Molecule Synthetic incorporated RNA substrate sequence
gatctgagcctgggagct

SEQ ID NO: 12 - DNA, 18 bp, Artificial Sequence
  Description of Artificial Sequence Synthetic incorporated RNA substrate sequence
agctcccaggctcagatc

SEQ ID NO: 13 - DNA, 18 bp, Artificial Sequence
  Description of Artificial Sequence Synthetic DNARNA substrate sequence
gatctgagcctgggagct

SEQ ID NO: 14 - DNA, 20 bp, Artificial Sequence
  Description of Artificial Sequence Synthetic primer
ccggtgagaagcgcagtcgg

SEQ ID NO: 15 - DNA, 18 bp, Artificial Sequence
  Description of Artificial Sequence Synthetic primer
ttcggattcccgcagtgt

SEQ ID NO: 16 - DNA, 20 bp, Artificial Sequence
  Description of Artificial Sequence Synthetic primer
tgccaggaagccaaatgagt

SEQ ID NO: 17 - DNA, 20 bp, Artificial Sequence
  Description of Artificial Sequence Synthetic primer
gggtgtccttaggcagaaca

SEQ ID NO: 18 - DNA, 20 bp, Artificial Sequence
  Description of Artificial Sequence Synthetic primer
cctctcgcccttagaaatcg

SEQ ID NO: 19 - DNA, 20 bp, Artificial Sequence
  Description of Artificial Sequence Synthetic primer
cccaagcccgaccccgagta

SEQ ID NO: 20 - DNA, 21 bp, Artificial Sequence
  Description of Artificial Sequence Synthetic primer
tcactttcccccctttatcca

SEQ ID NO: 21 - DNA, 19 bp, Artificial Sequence
  Description of Artificial Sequence Synthetic primer
tccctcttggcaacatcca

SEQ ID NO: 22 - DNA, 20 bp, Artificial Sequence
  Description of Artificial Sequence Synthetic primer
atggaaacctgtttggcttg

SEQ ID NO: 23 - DNA, 19 bp, Artificial Sequence
  Description of Artificial Sequence Synthetic primer
tagacgctcctggaacctc

SEQ ID NO: 24 - DNA, 18 bp, Artificial Sequence
  Description of Artificial Sequence Synthetic dsRNA substrate sequence
gatctgagcctgggagct

SEQ ID NO: 25 - DNA, 18 bp, Artificial Sequence
  Description of Artificial Sequence Synthetic dsDNA substrate sequence
gatctgagcctgggagct

(19) United States
(12) Patent Application Publication
(10) Pub. No.: US 2021/0003580 A1
(43) Pub. Date: Jan. 7, 2021

(54) METHODS OF DETECTION AND TREATMENT OF HYPER TRANSCRIPTION DISEASES

(71) Applicant: BOARD OF REGENTS OF THE UNIVERSITY OF TEXAS SYSTEM, Austin, TX (US)

(72) Inventors: Alexander James BISHOP, Houston, TX (US); Aparna GORTHI, Houston, TX (US); July Carolina ROMERO, Houston, TX (US); Liesl A. LAWRENCE, Houston, TX (US)

(21) Appl. No.: 15/733,349

(22) PCT Filed: Jan. 9, 2019
(86) PCT No.: PCT/US2019/012875

§ 371 Date: Jul. 8, 2020

Related U.S. Application Data
(60) Provisional application No. 62/615,339, filed on Jan. 9, 2018.

(51) Int. Cl. G01N 33/574

(52) U.S. Cl. CPC G01N 33/57496; A61K 45/06

(57) ABSTRACT

Described here are methods of treatment of hyper transcription diseases in patients by administering a combination of an inhibitor of Ataxia Telangiectasia and Rad3 related Serine/Threonine protein kinase and an inhibitor of poly (ADP-ribose) polymerase (1). Described here are methods of treatment of hyper transcription diseases in patients by administering a combination of an inhibitor of Checkpoint kinase (1) and an inhibitor of poly (ADP-ribose) polymerase (1). Also provided are methods for diagnosing hyper transcription diseases in a subject by analyzing a tissue sample from the subject for changes in the plurality of R-loops or the phosphorylation levels of RNA polymerase II.

### CROSS-REFERENCE TO RELATED APPLICATIONS

This application claims the benefit of U.S. Provisional Application No. 62/615,339, filed Jan. 9, 2018, which is incorporated by reference herein in its entirety.

### GOVERNMENT LICENSE RIGHTS

This invention was made with government support under grant number CA148724 awarded by the National Institutes of Health. The government has certain rights in the invention.

### FIELD

This disclosure relates to compositions and methods of detection and treatment of hyper transcription diseases.

### BACKGROUND

Diseases associated with high levels of transcription include a variety of cancers, such as Ewing sarcoma, acute myeloid leukemia and breast cancer as well as myelodysplastic syndrome and possibly Alzheimer's and amyotrophic lateral sclerosis. Ewing sarcoma (EwS) is a heterogeneous family of highly malignant, undifferentiated mesenchymal origin tumors that mainly affect children and young adults. Current treatment is multimodal, incorporating surgery, radiation, and (neo-) adjuvant chemotherapy including etoposide. Though systemic polychemotherapy has improved overall survival for patients with localized disease, the long-term ramifications of this aggressive regime are significant. Further, chemoresistant and metastatic patients have poor prognosis (<20% survival) and lack alternative therapeutic options.

### SUMMARY

Compounds and methods addressing the shortcomings of the art are disclosed here, which may provide additional or alternative advantages. Described herein are compounds, compositions, and methods for treatment of hyper transcription diseases. Certain embodiments include methods of detecting hyper transcription diseases.

Ewing sarcoma (EwS) is an aggressive pediatric bone and soft tissue cancer. It is driven by a fusion oncogene involving the EWSR1 gene and the Friend leukemia integration 1 transcription factor (FLI1), predominantly EWS-FLI1. EwS displays alterations in damage-induced transcription regulation and an accumulation of R-loops. An increase in replication stress but impaired homologous recombination was detected. An enriched interaction between breast cancer type 1 susceptibility protein (BRCA1) and the elongating transcription machinery was demonstrated as a possible underlying cause for impaired recombination. Transcription profiling of EwS and its putative cells of origin reveal high expression of genes, including heterotrimeric type II ribonuclease H enzyme (RNASEH2), Flap endonuclease 1 (FEN1) and Fanconi Anemia pathway, which compensate for EWS-FLI1 induced toxicity. A novel role for EWSR1 in transcriptional response to damage, suppression of R-loops, and promotion of homologous recombination was determined. Disclosed here are methods of detection of R-loops or phosphorylation status of RNA polymerase II as a biomarker for hyper transcription diseases, such as EwS. In certain embodiments, the method involves an immunohistochemical assay for detecting R-loops.

Disclosed here are methods of treatment of cancer in a subject by administering the subject with a therapeutically effective amount of an inhibitor of Ataxia Telangiectasia and Rad3 related Serine/Threonine protein kinase (ATR) and an inhibitor of poly (ADP-ribose) polymerase 1 (PARP1) or pharmaceutical acceptable salts thereof. Also disclosed here are methods for diagnosing and treating cancer in a subject. An embodiment of the method includes the steps of analyzing a tissue sample from the subject for presence or absence of a plurality of R-loops, and if the plurality of R-loops is detected, the subject is diagnosed with the cancer. The method further includes the step of administering the subject with a therapeutically effective amount of an inhibitor of ATR and an inhibitor of PARP1 or pharmaceutical acceptable salts thereof to the diagnosed subject. In another embodiment of a method for diagnosing and treating cancer in a subject, the tissue sample from the subject is analyzed for phosphorylation levels of RNA polymerase II. If an increase in phosphorylation levels of RNA polymerase II is detected, the subject is diagnosed with the cancer. The method further includes the step of administering such a subject with a therapeutically effective amount of an inhibitor of ATR and an inhibitor of PARP1 or pharmaceutical acceptable salts thereof to the diagnosed subject. Disclosed here are methods of stratifying patients by immunohistochemical assays to assess relative levels of R-loops and phosphorylated RNA polymerase II. Disclosed here are methods of treating Ewing sarcoma tumors using combination of an ATR inhibitor and a PARP1 inhibitor.

Disclosed here are methods of treatment of cancer in a subject by administering the subject with a therapeutically effective amount of an inhibitor of Checkpoint kinase (CHK1) and an inhibitor of poly (ADP-ribose) polymerase 1 (PARP1) or pharmaceutical acceptable salts thereof. Also disclosed here are methods for diagnosing and treating cancer in a subject. The method includes the steps of analyzing a tissue sample from the subject for the presence or absence of a plurality of R-loops, and if the plurality of R-loops is detected, the subject is diagnosed with the cancer. The method further includes the step of administering the subject with a therapeutically effective amount of an inhibitor of CHK1 and an inhibitor of PARP1 or pharmaceutical acceptable salts thereof to the diagnosed subject. In another embodiment of a method for diagnosing and treating cancer in a subject, the tissue sample from the subject is analyzed for phosphorylation levels of RNA polymerase II. If an increase in phosphorylation levels of RNA polymerase II is detected, the subject is diagnosed with the cancer. The method can also include the step of administering such a subject with a therapeutically effective amount of an inhibitor of CHK1 and an inhibitor of PARP1 or pharmaceutical acceptable salts thereof to the diagnosed subject. Disclosed here are methods of stratifying patients by immunohistochemical assays to assess relative levels of R-loops and phosphorylated RNA polymerase II. Disclosed here are methods of treating Ewing sarcoma tumors using combination of a CHK1 inhibitor and a PARP1 inhibitor. Numerous other aspects, features and benefits of the present disclosure may be made apparent from the following detailed description taken together with the formulae and tables.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIGS. 1A and 1B list the control cell lines and the EwS cell lines used in the experiments described in this application, in accordance with various embodiments.

FIGS. 2A-2E are graphical representations displaying cell viability measured after 72 hours exposure to etoposide (FIG. 2A), Zeocin (radiomimetic, (FIG. 2B)), Doxorubicin (Topoisomerase II poison, (FIG. 2C)), Mitomycin C (crosslinking agent, (FIG. 2D)), Hydroxyurea (replication stressor, (FIG. 2E)) and Methyl methane sulfonate (MMS) (alkylating agent, (FIG. 2F)).

FIG. 3A is a graphical representation demonstrating that EWS-FLI1 knockdown using siRNA against EWS-FLI1 (siFLI1) alleviated the cytotoxicity of TC32 in response to etoposide compared to cells transfected with scrambled siRNA (siCtrl) 24 hours prior to treatment. FIG. 3B is a bar graph showing level of cell death caused due to knockdown of EWS-FLI1 alone in TC32 cells. FIG. 3C is a photograph of an immunoblot, where the top right panel shows extent of knockdown (approximately 60% decrease) in EWS-FLI1 protein.

FIG. 4A is a graphical representation of cell viability of U2OS cells transfected either with empty vector (EV) or EWS-FLI1 (blue line) for 24 hours prior to etoposide exposure for further 48 hours. FIG. 4B is a photgraph of an immunoblot showing the transfection efficiency of empty vector (EV) or EWS-FLI1.

FIGS. 5A and 5B are box-and-whisker plots depicting the IC50 levels of etoposide and mitomycin, respectively, in EWS-FLI1 mutant versus pan-cancer cell lines.

FIG. 6A is a heatmap of basal gene expression profile in control and EwS cell lines after hierarchical clustering. FIG. 6B is a table capturing the top enriched pathways from Gene Set Enrichment Analysis (GSEA) of the differences between EwS and control cell lines. FIGS. 6C-6F are the relevant GSEA signature plots. FIGS. 6G-6I are illustrations of a cross-screen pathway comparison of top survival hits from RNAi screens in Drosophila Kc167 cells exposed to MMS, bleomycin or etoposide, respectively.

FIG. 7A is a heatmap of genes that are at least 2-fold altered in control (IMR90) cells in response to damage but not in the EwS cells (sorted by gene expression in IMR90). FIG. 7B presents tables of genes that are upregulated in IMR90 following treatment by etoposide.

FIG. 8 is a graphical representation depicting the degree of inhibition of phospho-Ser2 deposition by CDK9 kinase activity by either buffer (vehicle) or recombinant EWSR1/FUS proteins on two independent substrates (CDKtide-shorter triheptad repeat peptide, or CTD—the C terminal domain of RNAPII).

FIG. 9A is a graphical representation of the effects of inhibition of CDK9 kinase activity on RNAPII CTD fragment under various treatments—vehicle treated, staurosporine (a chemical pan-kinase inhibitor), recombinant FUS protein or EWSR1 protein. FIG. 9B is a photograph of an immunoblot depicting the phosphorylation of RNAPII CTD fragment used as the substrate in FIG. 9A. Recombinant EWSR1 and hypophosphorylated RNAPII are also displayed. FIG. 9C is a graphical representation of the effects of the inhibition of CDK9 kinase activity on a smaller tri-heptad repeat of RNAPII CTD (CDKtide, 21-mer) under the same conditions as in FIG. 9A.

FIG. 10A is a graphical representation of the percentage of inhibition of CDK7 kinase activity (phosphorylation on Ser5 of RNAPII CTD repeats) with or without EWSR1 on the two substrates. FIG. 10B is a graphical representation of the effects of inhibition of CDK9 kinase activity by buffer (vehicle) or recombinant EWS-FLI1 protein on the two RNAPII CTD substrates. FIG. 10C is a photograph of an immunoblot confirming the kinase activity measured by the assay. The blots show amount of recombinant EWS-FLI1 protein and its effect on the phosphorylation of the bigger CTD domain fragment (45kDa). Hypophosphorylated RNAPII is also indicated. FIG. 10D is a graphical representation of the effects of inhibition of CDK7 kinase activity by buffer (vehicle) or recombinant EWS-FLI1 protein on the two RNAPII CTD substrates.

FIG. 11A is a photograph of an immunoblot depicting levels of phosphorylated Ser2/Ser5 at CTD of RNAPII in U2OS cells with and without EWSR1 knockdown. FIG. 11B is a photograph of an immunoblot depicting levels of phosphorylated Ser2/Ser5 at CTD of RNAPII in control IMR90 cells versus TC32 cells. FIG. 11C is a photograph of an immunoblot depicting levels of phosphorylated Ser2/Ser5 at CTD of RNAPII in TC32 cells with and without EWS-FLI1 knockdown.

FIG. 12 is a photograph of an immunoblot depicting phosphorylated RNAPII in a panel of EwS cell lines.

FIG. 13A is a graphical representation of a cytotoxicity profile of control, EwS and EWSR1-depleted cells exposed to increasing doses of camptothecin, measured 72 hours after treatment. FIG. 13B is a graphical representation of a cytotoxicity profile of control, EwS and EWSR1-depleted cells exposed to increasing doses of DRB, measured 72 hours after treatment. FIG. 13C is a box-and-whisker plots depicting the IC50 levels of etoposide or mitomycin in EWS-FLI1 mutant (15 cell lines) versus pan-cancer (132 cell lines).

FIG. 14 is a graphical representation of the results from the transcription restart assay measured in in control IMR90 cells versus TC32 cells.

FIG. 15 is a graphical representation of the results from the transcription restart assay measured in U2OS cells transfected with either scrambled siRNA or EWSR1 siRNA.

FIG. 16A is a histogram of the percentage occupancy of RNAPII in the promoter regions (<=1000, <=2000 and <=3000 bp upstream of the transcription start site) in RNAPII ChIP sample of IMR90 cells. FIG. 16B is a histogram of the percentage occupancy of RNAPII in the promoter regions (<=1000, <=2000 and <=3000 bp upstream of the transcription start site) in RNAPII ChIP sample of EWS502 cells. FIG. 16C is a histogram of the percentage occupancy of RNAPII in the promoter regions (<=1000, <=2000 and <=3000 bp upstream of the transcription start site) in RNAPII ChIP sample of TC32 cells.

FIG. 17A is a graphical representation of the quantification of R-loops detected using S9.6 antibody on dot blot of genomic DNA from IMR 90, TC32, EWS502, and CHLA10 cells. FIGS. 17B-17D are representative dot blot indicating levels of RNA:DNA hybrids or single-stranded DNA (ssDNA). The dot blots of FIGS. 17B and 17C were probed with S9.6 antibody. In FIG. 17B, Panel 1 contains genomic DNA and in FIG. 17C, Panel 2 is genomic DNA that had been further treated with RNaseH enzyme. In FIG. 17D, Panel 3 has denatured genomic DNA that was probed with ssDNA antibody. Each dot serves as a technical replicate. FIG. 17E is a graphical representation of quantification from a dot blot measuring levels of RNA:DNA hybrids in DNA harvested from TC32 that have been transfected either with an empty vector (EV) or RNASEH1 (RNH1). R-loop signal was normalized to its loading control (ssDNA). FIG. 17F is a photograph of an immunoblot that indicates the RNASEH1 transfection efficiency. FIG. 17G is a graphical representation of quantification of RNA:DNA hybrids in DNA harvested from TC32 cells that have been depleted of EWS-LI1 (siFLI1). FIG. 17H is a graphical representation of the quantification of R-loops detected using S9.6 antibody on dot blot of genomic DNA from U2OS cells transfected with either siRNA against EWSR1 or EWS-FLI1 expression vector. FIG. 171 is a graphical representation of quantification of RNA:DNA hybrids in U2OS cells with shRNA mediated depletion of EWSR1. FIG. 17J is a schematic of the EWS-FLI1 R2L2 construct. Arginine residues 383 and 386 (indicated by black vertical bars) in EWS-FLI1 are converted to Leucine to render the fusion oncogene deficient in DNA binding. FIG. 17K is a graphical representation of quantification of RNA:DNA hybrids in U2OS cells expressing either empty vector (EV), EWS-FLI1 or EWS-FLI1 R2L2.

FIG. 18A is a graphical representation of the fold change in R-loop signal after damage (etoposide, 6 hours) in control vs. EwS cells. FIG. 18B is a graphical representation of the fold change in R-loop signal after damage (etoposide, 6 hours) in U2OS cells with either EWSR1 depletion or EWS-FLI1 expression.

FIGS. 19A-19D present immunofluorescence images of cells stained with DAPI for the nucleus. FIGS. 19E-19H present immunofluorescence images of cells stained with S9.6 for the R-loops. FIGS. 19I-19L present immunofluorescence images of cells stained with nucleolin for the nucleoli. FIGS. 19M-19P present immunofluorescence images of cells that merges the staining from DAPI (nucleus), S9.6 (R-loops) and nucleolin (nucleoli). Scale bar (white line) provided in FIG. 19M represents 25 μm.

FIG. 20 is a graphical representation of the quantification of nucleoplasmic R-loops in the immunofluorescence images.

FIG. 21 is a graphical representation of the quantification of cell viability of U2OS cells measured using Celltiter-glo after 48 hours of transfection with EWS-FLI1 or RNASEH1 or both. Error bars represent SE from 8 independent transfections.

FIG. 22A is a flow chart of the algorithm used to determine consensus DRIP regions for each sample. FIG. 22B is a flow diagram of methodology designed to evaluate statistical significance of correlation between DRIP-Seq, ChIP-Seq (BRCA1 and EWS-FLI1) and expression data using Kolmogorov-Smirnov (KS) test.

FIG. 23 is a schematic illustration of the Kolmogorov-Smirnov Test.

FIG. 24 is a schematic representation of a 35 kb region surrounding the SON gene.

FIGS. 25A and 25B are chromosome wide maps of R-loop sites in IMR90 and TC32 untreated DRIP samples as determined by the MACS2 program. FIGS. 25C-25E are representative pie charts depicting the differences in enrichment of DRIP between IMR90 and the EwS cell line EWS502. Technical biases during IP and sequence alignment can be evaluated based on the Input genome. FIG. 25F is a representation of an 8 Mbp region of chromosome 8 surrounding the HSF1/DGAT genes.

FIG. 26A is a graphical representation of the quantification of DRIP (coverage of DRIP region multiplied by reads in that region) across all the samples. Y-axis is graphed in logarithmic scale. FIGS. 26B-26C are graphical representations of the fold enrichment of quantitative-PCR (qPCR) product from ChIP experiments done with RNAPII antibody in control and EwS cell lines. FIG. 26D is a photograph of representative agarose gel blots testing validity of DRIPs by PCR at a well-known DRIP site in the target gene APOE. SNRPN serves as a negative control.

FIG. 27A is a representative heatmap of genes (whole genome) centered around transcription start site (TSS) ordered by average expression of EwS cells. FIG. 27B is a probability density graph plotted with a Gaussian smoothing kernel of the distribution of DRIP peaks and EWS-FLI1 ChIP peaks at EWS-FLI1 bound genes relative to uniform distribution. FIG. 27C is a photograph of immunoblots showing increased levels of phospho-ATR, phospho-CHK1 and phospho-RPA2 in TC32 compared to IMR90.

FIG. 28A is a probability density plot of the distribution of DRIP peaks and expression profile at the corresponding peaks across all genes relative to uniform distribution. In FIG. 28B, the data was sorted by DRIP sites in EWS502.

FIG. 29A is a photograph of immunoblots depicting a panel of EwS cell lysates evaluated for proteins that are activated (phosphorylated) in response to replication stress (ATR/CHK1/RPA2). FIG. 29B is a graphical representation of the quantification of the immunoblots from two independent experiments of FIG. 29A. FIG. 29C is a photograph of representative immunoblots evaluating decrease in the ATR kinase pathway activation upon overexpression of RNASEH1 in TC32 cells. FIGS. 29D and 29E are representations of DNA content analysis using flow cytometry on TC32 cells with and without RNASEH1 overexpression respectivly. FIG. 29F is a graphical representation of the cell viability plot displaying proliferation rate of TC32 cells with and without RNASEH1 overexpression over time after treatment with either vehicle or an ATR inhibitor (VE-821, ATRi). FIG. 29G is a graphical representation of the cell viability plot based on confluence measurements by IncuCyte showing increased sensitivity to ATR inhibitor in U2OS cells transfected with EWS-FLI1 compared to empty vector (EV) transfected cells.

FIG. 30A is a graphical representation of the cell viability response to ATR inhibitor (VE-821) in control, EwS and EWSR1 depleted U2OS cells. FIG. 30B is a graphical representation of the replication stress as quantified by evaluating number of stalled replication forks (Schematic, IdU label only) using DNA fiber spread method.

FIG. 31A is a set of immunofluorescence images taken at 40× magnification to detect number of DSBs (53BP1 foci) and IR-induced RAD51 foci. Scale bar (white line) provided at the bottom right represents 25 μm. FIG. 31B is a graphical representation of the quantification of number of cells with >5 53BP1 foci and FIG. 31C is a graphical representation of the quantification of number of cells with >5 RAD51 foci. n=3 sets of 80 nuclei each.

FIG. 32A is a schematic illustration of the DR-GFP construct integrated into U2OS cells used to measure HR. FIG. 32B is a graphical representation of HR frequency, measured as number of GFP positive cells (flow cytometry) with transient transfection of EWS-FLI1, EWS-ERG, EWSR1-full length (EWSR1) and EWSR1 N terminal domain only (EWSR1 N term) relative to empty vector (EV) DR-GFP U2OS cells. FIG. 32C is a graphical representation of the results from a DR-GFP assay used to evaluate effect of silencing EWSR1 with two independent siRNA on HR.

FIGS. 33A-33B are graphical representations of quantification of percentage of cells in the different phases of cell cycle with either knockdown of EWSR1 (FIG. 33A) or expression of EWS-FLI1 (FIG. 33B). FIG. 33C is a graphical representation of quantification of HR frequency following expression of either EWS-FLI1 or DNA binding mutant EWS-FLI1 R2L2. FIG. 33D is a graphical representation of quantification of HR frequency in DR-GFP U2OS cells transfected with either scrambled siRNA (siCtrl), FUS siRNA (siFUS) or TAF15 siRNA(siTAF15). FIG. 33E is a photograph of an immunoblot showing the levels of knockdown of FUS siRNA (siFUS) or TAF15 siRNA(siTAF15).

FIG. 34A is a graphical representation of RNA-seq data of BRCA1 transcript levels in EwS cell lines compared to IMR90. FIGS. 34B, 34C, 34D, and 34E are photographs of immunoblots demonstrating level of transfection of various siRNA and expression constructs used in FIGS. 35A-35C. FIG. 34F is a box-and-whisker plot depicting the IC50 levels of olaparib in EWS-FLI1 mutant versus just breast cancers or pan-cancer cell lines. FIG. 34G is a graphical representation of cell viability demonstrating the role of EWS-FLI1 in mediating exquisite sensitivity to olaparib in U2OS cells transfected either with the oncogene or empty vector (EV). FIGS. 34H, 34I, and 34J are photographs of immunoblots demonstrating level of transfection of various siRNA and expression constructs used in FIG. 35E. FIG. 34K is a photograph of an immunoblot depicting level of 53BP1 knockdown in TC32 for cell viability studies in FIG. 35F.

FIG. 35A is a graphical representation of measurements of HR frequency in DR-GFP U2OS cells expressing EWS-FLI1 with either siRNA against BRCA1 or overexpression of BRCA1 (transfection efficiency demonstrated in FIGS. 34B-34E). FIG. 35B is a graphical representation of the fold change in HR frequency in DR-GFP U2OS cells with EWSR1 depletion in combination with either siRNA against BRCA1 or BRCA1 overexpression. FIG. 35C is a graphical representation of the fold change in HR frequency in DR-GFP U2OS cells with EWSR1 overexpression in the presence of BRCA1 knockdown relative to siCtrl+EV transfected cells (transfection efficiency demonstrated in FIGS. 34H-34J). FIG. 35D is a cytotoxicity plot of IMR90 and EwS cells exposed to increasing doses of Olaparib. Cell viability was measured 72 hours post treatment. FIG. 35E is a graphical representation of the depletion of 53BP1 partially rescued the HR defect conferred by either EWS-FLI1 expression or EWSR1 knockdown in the DR-GFP assay. FIG. 35F is a graphical representation of the 53BP1 knockdown improved EwS (TC32) survival in response to damage.

FIG. 36A is a graphical representation of quantification of HR frequency in DR-GFP U2OS cells transfected with EWS-FLI1, RNASEH1 or both. FIG. 36B is a photograph of immunoblots of whole cell lysates and various subcellular fractions from U2OS cells with and without EWSR1 depletion. FIG. 36C is a photograph of immunoblots of chromatin and nuclear fractions of EwS and U2OS cell lines with and without etoposide treatment (2 hours). FIG. 36D is a photograph of immunoblots of IMR90 and EWS502 nuclear lysates with and without exposure to etoposide (2 hours). FIG. 36E is a photograph of immunoblots following co-immunoprecipitation with phosphorylated (Ser5) RNAPII in nuclear lysates from U2OS and TC32 with and without exposure to etoposide (2 hours).

FIG. 37A is a photograph of immunoblots showing equivalent levels of BRCA1 in whole cell lysates from control and EwS cells with and without etoposide treatment (2 hours). FIG. 37B is a photograph of immunoblots showing the differences in BRCA1 redistribution in subcellular fractions of U2OS or TC32 cells. FIG. 37C is a photograph of immunoblots showing BRCA1 and FLI1 in the nuclear and chromatin fractions of TC32 with and without EWS-FLI1 knockdown. FIG. 37D is a photograph of immunoblots showing immunoprecipitations with antibodies against BRCA1 or IgG in nuclear lysates of U2OS and TC32 with or without exposure to etoposide (2 hours). Input represents 10% of the amount used for immunoprecipitation. FIG. 37E is a graphical representation of the relative abundance of BRCA1 occupancy in DR-GFP U2OS cells transfected with either empty vector (EV, grey bars) or EWS-FLI1 (red bars) at various sites near the ISceI induced DSB. FIG. 37F is a heatmap representation of correlation between gene expression, RNAPII and BRCA1 binding sites, and DRIP locations across the entire genome of IMR90, TC32 and EWS502 cells ordered by DRIP loci centered around the transcription start site (TSS).

FIG. 38 is a heatmap showing a strong concordance between the ENCODE BRCA1 ChIP datasets from HeLa and human embryonic stem cells (hESC) and the ChIP data generated in-house (IMR90, TC32, EWS502 and U2OS).

FIG. 39A is a graphical representation of results from real-time qPCR analysis of BRCA-ChIP samples processed from the different control and EwS cell lines with and without etoposide treatment, using primers within FEN1. FIG. 39B is a diagrammatic representation of gene expression (red tracks), R-loop sites (black and grey tracks) and BRCA1 binding sites (blue tracks) across the FEN1 gene clearly demonstrated the enrichment of R-loops and BRCA1 in the region amplified by the primers in FIG. 39A. FIG. 39C is a graphical representation of results from qPCR analysis similar to FIG. 39A with primers amplifying a region within the PARP8 gene. FIG. 39D is a graphical representation of results from rqPCR analysis as in FIG. 39A with primers targeting a well-known R-loop region within APOE gene. FIG. 39E is a diagrammatic representation of gene expression (red tracks), R-loop sites (black and grey tracks) and BRCA1 binding sites (blue tracks) across the APOE gene again demonstrated the enrichment of R-loops and BRCA1 in the region amplified by the primers in FIG. 39D. FIG. 39F is a graphical representation of results from qPCR analysis as in FIG. 39C but using RNAPII ChIP samples indicating levels of RNAPII occupancy among the different cell lines with and without treatment. FIG. 39G is a photograph of agarose gel blots evaluating amplicons generated using EWS502 DRIPs with primers against FEN1 and PARP8 genes.

FIGS. 40A and 40B are graphical representations of the distribution of RNAPII abundance bound to DNA across the genome for IMR90 and TC32, respectively. FIGS. 40C and 40D are graphical representations of the distribution of BRCA1 abundance across the genome for IMR90 and TC32, respectively. FIGS. 40E and 40F are scatter plots representing the correlation of RNAPII (FIG. 40E) and BRCA1 (FIG. 40F) peak heights between TC32 and IMR90. FIGS. 40G and 40H are scatter plots representing the relationship between co-localized BRCA1 and RNAPII peaks as a function of BRCA1 peak height (X-axis) and level of expression of the gene associated with these binding sites (Y-axis) in IMR90 and TC32 cells, respectively.

FIG. 41A is a heatmap representing genome-wide localization of RNAPII Binding, BRCA1 binding and R-loops centered around the transcription start site (TSS). FIGS. 41B-41E are KS plots to demonstrate enrichment of BRCA1, RNAPII and R-loops at highly expressed genes. These KS plots demonstrate empirical distribution of the top 13.8% of DRIP and ChIP peaks and higher expression relative to uniform distribution. FIG. 41F is an illustration of the data as sorted by BRCA1 ChIP, n=3,066 genes.

FIG. 42A is a probability density plot of the distribution of EWS-FLI1 binding peaks as a function of underlying sequence characteristic across all the 1762 unique genes (associated with the EWS-FLI1 binding sites) relative to uniform distribution. FIG. 42B presents heatmaps of gene expression, RNAPII, BRCA1 and R-loop sites at EWS-FLI1 bound genes (1762) centered on EWS-FLI1 peaks. FIG. 42C is a set of KS plots depicting enrichment of BRCA1, RNAPII and R-loops within the top 7.5% of EWS-FLI1 binding sites. Data was ordered by distance to TSS.

FIG. 43A is a heatmap of gene expression, BRCA1 and R-loop sites at genes associated with EWS-FLI1 binding proximal (<10 kb) to its TSS (n=284 sites or 280 unique genes; Table 12). FIG. 43B is a set of KS plots depicting enrichment of BRCA1 and R-loops within the top 35% of promoter-proximal EWS-FLI1 binding sites.

FIG. 44A is a diagrammatic representation of the BRCA1 interactions in normal cells. FIG. 44B is a diagrammatic representation of the BRCA1 interactions in Ewing sarcoma cells.

FIG. 45A is a graphical representation of the upregulation at transcript levels of genes in the RNASEH2A complex. FIG. 45B is a graphical representation of the upregulation at transcript levels of genes in the RNASEH2B complex. FIG. 45C is a graphical representation of the upregulation at transcript levels of genes in the RNASEH2C complex. FIG. 45D is a graphical representation of the upregulation at transcript levels of FEN1. FIGS. 45E-45N are graphical representations of the upregulation at transcript levels of genes in the Fanconi Anemia pathway: FANCA (FIG. 45E); FANCB (FIG. 45F); FANCC (FIG. 45G); BRCA2 (FIG. 45H); FANCD2 (FIG. 45I); FANCE (FIG. 45J); FANCG (FIG. 45K); FANCL (FIG. 45L); FANCI (FIG. 45M); and FANCM (FIG. 45N).

FIG. 46A is a photograph of an immunoblot showing increased basal expression of RNASEH2A in EwS cell lines. FIG. 46B is a graphical representation of the levels of RNASEH2A activity in hydrolyzing RNA in RNA:DNA hybrids measured using a fluorometric assay with whole cell lysates and substrate as indicated in the schematic. Signals were normalized relative to IMR90 activity. FIG. 46C is a graphical representation of cell viability curves in response to increasing doses of RNASEH2 inhibitor R11 measured 72 hours after addition of the drug. FIG. 46D is a photograph of an immunoblot showing increased levels of FEN1 in EwS cells. FIG. 46E is a graphical representation of cell viability measured 72 hours after incubation with an inhibitor of FEN1 endonuclease activity caused significant cell death in EwS. R-loops were evaluated as a potential substrate for FEN1. FIG. 46F is a graphical representation of R-loop signal intensity in genomic DNA of cells that were treated with 10 μM FEN1 inhibitor (FEN1i) for 6 hours, as measured by dot blot assay and quantified using ImageJ software. FIG. 46G is a photograph of an immunoblot showing levels and activation of proteins involved in FA pathway in whole cell lysates of control and EwS (TC32); the mono-ubiquitination of FANCI and FANCD2 are indicated by black triangles. FIG. 46H is a graphical representation of the knockdown of several FA genes induced significantly more cell death in TC32 compared to IMR90 measured 72 hours after knockdown. FIGS. 46I-46L are box and whisker plots depicting gene expression levels of upregulated factors across normal tissues (triplicate) and 32 primary EwS tumor samples. FIG. 46M is a set of representative immunohistochemistry images showing R-loops (S9.6) staining across a sarcoma tissue microarray.

FIG. 47A is a graphical representation of gene expression of RNASEH2A across independently generated datasets. FIG. 47B is a graphical representation of gene expression of FEN1 across independently generated datasets. FIG. 47C is a graphical representation of gene expression of FANCI across independently generated datasets. FIG. 47D is a graphical representation of gene expression of FANCD2 across independently generated datasets.

FIGS. 48A-48C are photographs of immunohistochemical images depicting R-loop staining by S9.6 antibody on sections derived from fixed TC32 cell pellets. Sections were either treated with buffer (FIG. 48A), RNaseH (FIG. 48B) or RNaseA (FIG. 48C) after antigen retrieval. FIGS. 48D-48O are photographs of Representative IHC images from a pan-sarcoma tissue microarray.

FIG. 49 is an immunoblot showing levels of c-AID (AICDA gene) enzyme across a panel of control and EwS whole cell lysates.

### DETAILED DESCRIPTION

In the following description, numerous specific details are set forth in order to provide a thorough understanding of the various embodiments. In other instances, well-known processes and methods may not be described in particular detail in order not to unnecessarily obscure the embodiments described here. Additionally, illustrations of embodiments here may omit certain features or details in order to not obscure the embodiments described here.

In the following detailed description, reference is made to the accompanying formulae and tables that form a part of the specification. Other embodiments may be utilized, and logical changes may be made without departing from the scope of the disclosure. Therefore, the following detailed description is not to be taken in a limiting sense.

Disclosed here are methods of treating Ewing sarcoma tumors using combination of an ATR inhibitor and a PARP1 inhibitor. Disclosed here are methods of treating Ewing sarcoma tumors using combination of a CHK1 inhibitor and a PARP1 inhibitor. PARP1 inhibition can impact BRCA1 deficiency resultant from mutation or lack of expression, resulting in a DNA repair deficiency. PARP1 inhibition either results in trapping PARP1 on DNA or blocks its ability to bind DNA. During DNA replication, this allows conversion of single strand breaks into double strand DNA breaks that require BRCA1-promoted homologous recombination to repair appropriately. Problems in DNA integrity can result in problems during DNA replication, which induces replication stress response (ATR, CHK1, TOPBP1, ETAA1, VCP/p97 pathway). Use of inhibitors targeting components of this pathway, such as ATR, CHK1 and VCP, can exasperate replication stress and is deleterious to cells with high levels of lesions in their DNA.

Gene changes that alter control of RNA polymerase II transcription activity result in both increased problems in transcription progression and trapping of the BRCA1 protein with the transcription complex. BRCA1 normally interacts with phosphorylated RNA polymerase II. But, upon damage RNA polymerase II globally loses phosphorylation and BRCA1 is released to promote homologous recombination at double strand breaks. With high levels of transcription, there is increased opportunity for nascent RNA to bind template DNA, producing a heterodimer that is more thermodynamically stable than DNA:DNA homodimer. This event results in a structure consisting of a RNA:DNA hybrid and a displaced single stranded non-template DNA strand, or an “R-Loop.” These R-loops present a DNA lesion that causes replication stress. As such, dysregulating transcription results in both replication stress and a homologous recombination defect, which are targeted using inhibitors of the replication stress response and PARP1 inhibitors for synergistic effect. Mutations that can cause transcriptional dysregulation are mutation, deletion (even heterozygous deletion), fusion and sometimes amplification of genes such as EWSR1, FUS, TAF15, CREBBP, c-MYC, n-MYC, CDK7, CDK9, and CDK12. Either loss of EWSR1 or expression of the EWSR1-FLI1 fusion protein (found in Ewing sarcoma) results in high levels of RNA polymerase II phosphorylation, accumulation of R-loops, and a homologous recombination repair defect through BRCA1 protein trapping. Based on known functions, amplification or increased expression of c-MYC, n-MYC, CDK7, CDK9 and CDK12 is expected to result in the same phenotype. Disclosed here are combinations of inhibitors of ATR and PARP1 for the treatment of hyper transcription diseases. Disclosed here are combinations of inhibitors of CHK1 and PARP1 for the treatment of hyper transcription diseases. Diseases associated with high levels of transcription include a variety of cancers, such as Ewing sarcoma, acute myeloid leukemia and breast cancer as well as myelodysplastic syndrome and possibly Alzheimer's and amyotrophic lateral sclerosis. Those with a replicative component are susceptible to the combinations of inhibitors of ATR and PARP1.

EwS ensues from a chromosomal translocation, predominantly t(11;22)(q24;q12), that fuses the N terminal transactivation domain of the constitutively expressed Ewing sarcoma break point region 1 (EWSR1) gene with the C terminal DNA binding domain of the rarely expressed friend leukemia integration 1 (FLI1) gene. The resultant oncogene EWS-FLI1 is a potent transcription factor that drives tumor initiation and maintenance, while the second EWSR1 allele is usually intact. The N-terminal domain of EWSR1 is involved in oncogenic translocations in several cancers, with over twenty fusion partners. Wild-type EWSR1 is an RNA binding protein affecting RNA metabolism presumably through its regulation of RNA polymerase II (RNAPII) and coupling with the splicing machinery. In addition to a gain-of-function, prior studies also indicate a dominant-negative role for EWS-FLI1 over EWSR1 function, especially in the context of splicing regulation. EWSR1 is also involved in genome stability, with in vivo knockout models displaying ionizing radiation (IR) hypersensitivity and mitotic/meiotic defects. EWSR1 may play a direct role in genome surveillance as indicated by its ability to bind single-stranded DNA and promote homologous pairing. Aberrant regulation of transcription is an important source of endogenous DNA damage. One consequence of altered transcriptional regulation is an R-loop. R-loops are stable structures involving an RNA:DNA heteroduplex and a displaced complementary single stranded DNA loop. R-loops are physiologically relevant for class-switch recombination, maintenance of unmethylated promoter CpG islands, and transcription termination. However, their accumulation has pathological consequences in terms of replication stress, hyper-recombination, and genome instability. An intricate network of processes coordinates R-loop metabolism. For example, RNA processing factors engage the nascent mRNA to prevent re-annealing with the DNA template, while nucleases such as RNASEH1/2 and RNA helicases such as senataxin facilitate their dissolution. Multiple DNA repair factors have been implicated in R-loop resolution, including breast cancer 1 and 2, early onset (BRCA1/BRCA2) genes in homologous recombination (HR). EWSR1, EWS-FLI1, and BRCA1 interact with RNAPII to regulate transcription, thus advocating a potential role for these proteins in R-loop accumulation. An embodiment of the method includes the steps of analyzing a tissue sample from the subject for presence or absence of a plurality of R-loops, and if the plurality of R-loops is detected, the subject is diagnosed with the cancer. The method further includes the step of administering the subject with a therapeutically effective amount of an inhibitor of ATR and an inhibitor of PARP1 or pharmaceutical acceptable salts thereof to the diagnosed subject. In an embodiment, the method further includes the step of administering the subject with a therapeutically effective amount of an inhibitor of CHK1 and an inhibitor of PARP1 or pharmaceutical acceptable salts thereof to the diagnosed subject. Disclosed here are methods of stratifying patients by immunohistochemical assays to assess relative levels of R-loops and phosphorylated RNA polymerase II.

The loss of EWSR1 function, due to either direct silencing or through interference by EWS-FLI1, results in widespread R-loop accumulation, increased replication stress, and HR deficiency. PARP1 inhibition is known to be synthetic lethal with BRCA1 deficiency, a phenotype shared by EwS. The HR defect in EwS results from retention of BRCA1 with transcriptional complexes. To counteract the detrimental consequences of EWS-FLI1 expression, EwS relies on the activity of ATR, RNASEH2, Fanconi Anemia pathway, and FEN1, all of which are previously unknown dependencies that may provide novel therapeutic targets as described here.

The cytotoxicity of a well-established panel of EwS cell lines (Table 1) was evaluated against a broad range of genotoxic agents to determine chemosensitivity. Table 1 describes the type of EWS-FLI1 fusion and the exons in EWSR1 and FLI1 where the breakpoint occurs for each of the cell lines used in the study.

TABLE 1
Cell Line Characteristics.

| Cell Line | Fusion Type | EWSR1 breakpoint | FLI1 breakpoint |
|---|---|---|---|
| TC32 | Typ | 7 | 6 |
| EWS502 | Typ | 7 | 6 |
| CHLA10 | Typ | 7 | 6 |
| CHLA2 | Typ | 7 | 6 |
| IMR90 | — | wild type | Very low expression |
| U2OS | — | wild type | Not expressed |

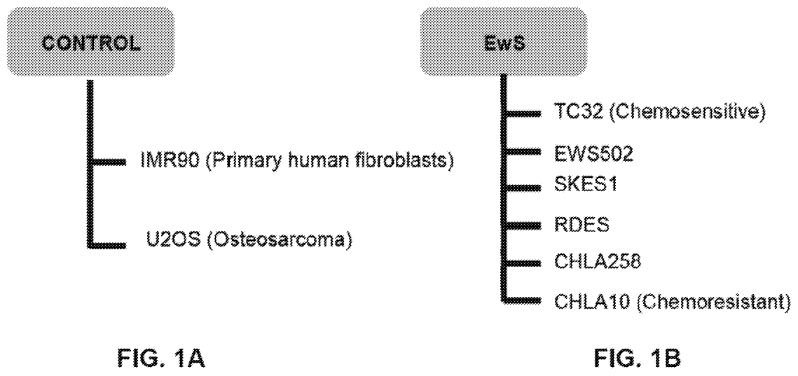

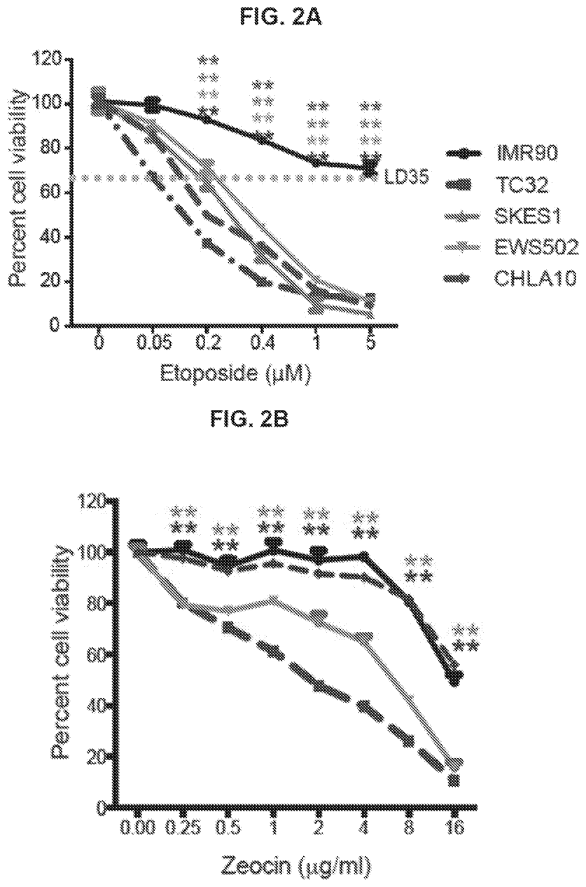

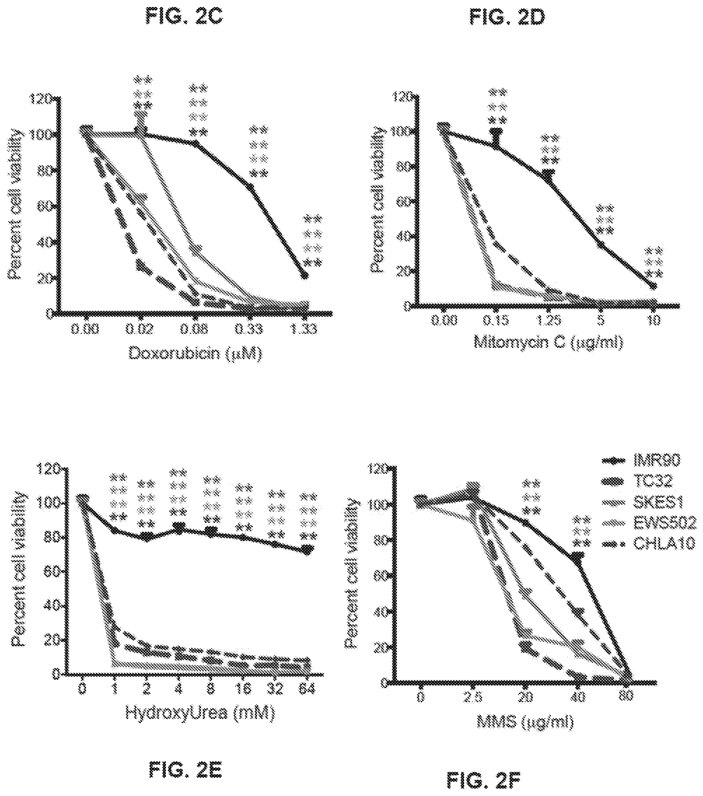

A primary fibroblast (IMR90) and a pediatric osteosarcoma cell line (U2OS) were used as controls (FIG. IA) in addition to six EwS cell lines (FIG. 1B). Compared to the controls, EwS cell lines were acutely sensitive to most forms of damage, including etoposide (topoisomerase II inhibitor) and hydroxyurea (replication stress) (FIGS. 2A-2E). FIGS. 2A-2E are graphical representations displaying cell viability measured after 72 hours exposure to etoposide, Doxorubicin (Topoisomerase II poison), Mitomycin C (crosslinking agent), Zeocin (radiomimetic), Hydroxyurea (replication stressor) and Methyl methane sulfonate (MMS, alkylating agent). Etoposide dose causing 35% lethality (LD35, indicated by the dotted gray line) was used for all further experiments unless otherwise indicated. EwS cell lines were significantly more sensitive to the wide range of DNA damaging agents. CHLA10 (a radioresistant cell line) demonstrated similar insensitivity to zeocin as the control cell line.

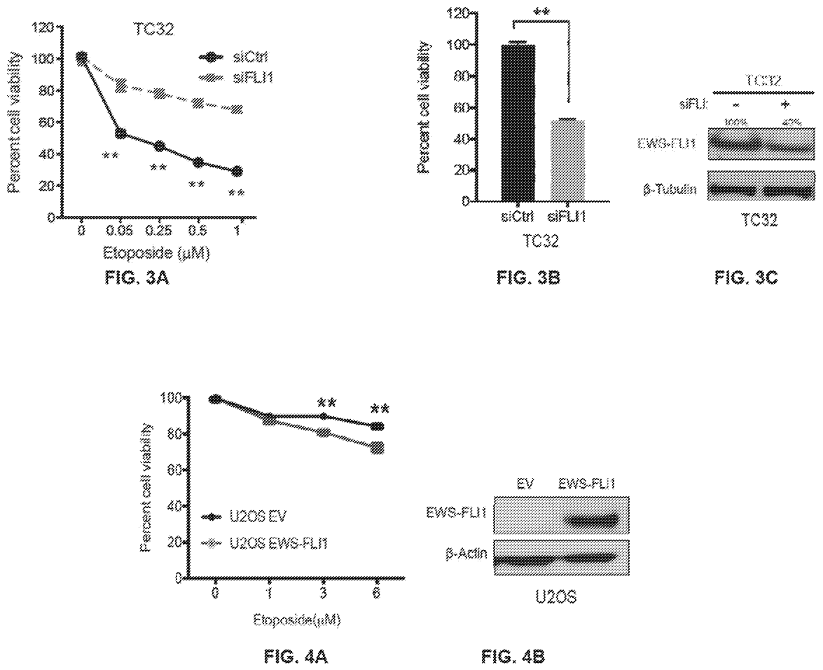

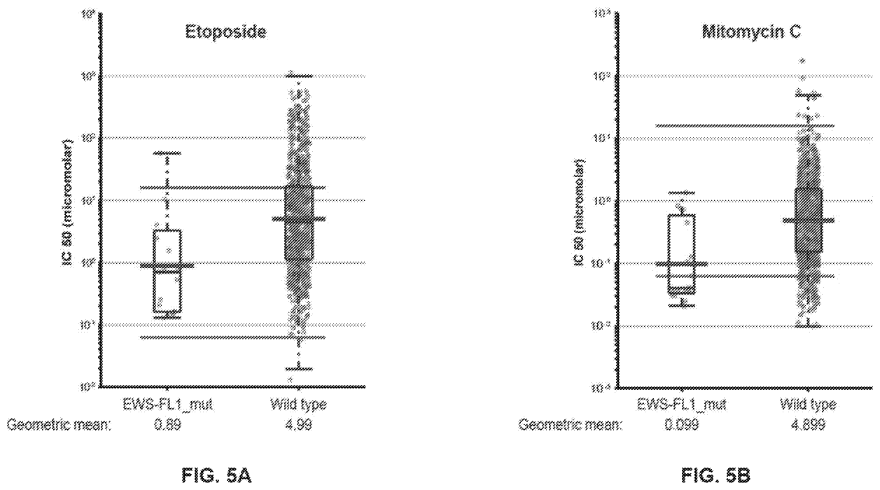

Importantly, EWS-FLI1 knockdown using siRNAs diminished etoposide toxicity (FIG. 3A) in spite of the toxicity incurred due to oncogene knockdown (FIGS. 3B and 3C), implicating a direct role of the fusion protein in mediating chemosensitivity. FIG. 3A is a graphical representation demonstrating that EWS-FLI1 knockdown using siRNA against EWS-FLI1 (siFLI1) alleviated the cytotoxicity of TC32 in response to etoposide compared to cells transfected with scrambled siRNA (siCtrl) 24 hours prior to treatment. FIG. 3B is a bar graph showing level of cell death caused due to knockdown of EWS-FLI1 alone in TC32 cells. FIG. 3C is a photograph of an immunoblot, where the top right panel shows extent of knockdown (approximately 60% decrease) in EWS-FLI1 protein. FIG. 4A is a graphical representation of cell viability of U2OS cells transfected either with empty vector (EV) or EWS-FLI1 (blue line) for 24 hours prior to etoposide exposure for further 48 hours. Transfection efficiency is indicated by the immunoblot, shown in FIG. 4B. FIGS. 5A and 5B are box-and-whisker plots depicting the IC50 levels of etoposide and mitomycin, respectively, in EWS-FLI1 mutant versus pan-cancer cell lines. The EWS-FLI1 mutant group contains 16 cell lines and the pan-cancer group contains 143 cell lines. The brown lines depict the range of screening concentrations of the drug. The red lines represent the geometric mean of the drug concentration. *P<0.05, **P<0.005, the colors of the * match that used for the cell line in the multi-cell line cytotoxicity plots and were tested against control. Line graphs display means with SE, n=4. Expression of EWS-FLI1 in U2OS resulted in a similar increase in chemosensitivity compared to cells transfected with empty vector (FIGS. 4A and 4B).

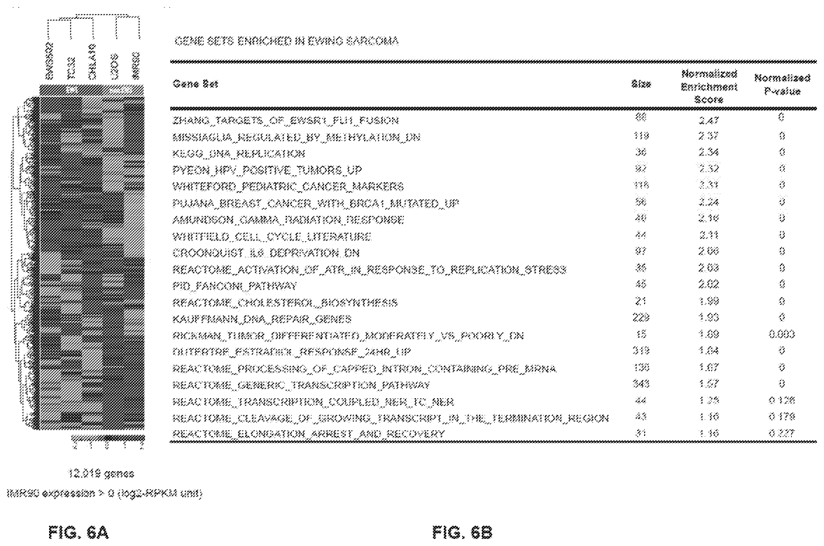

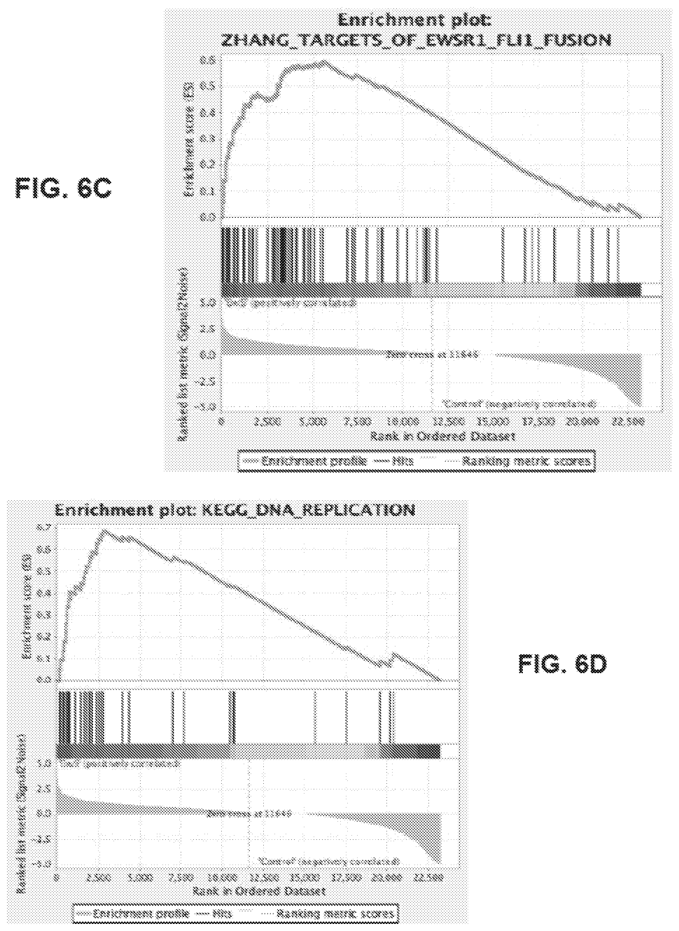

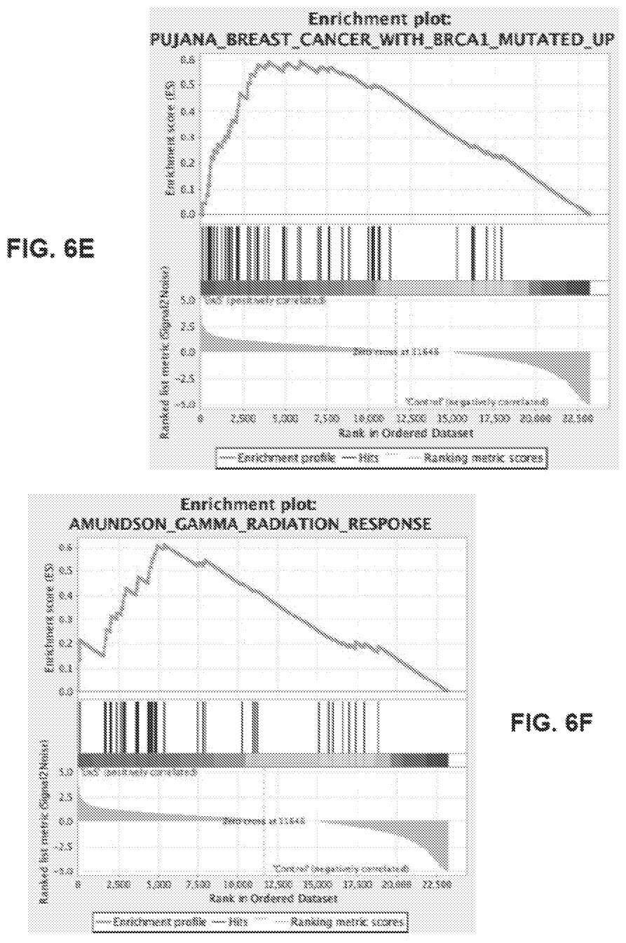

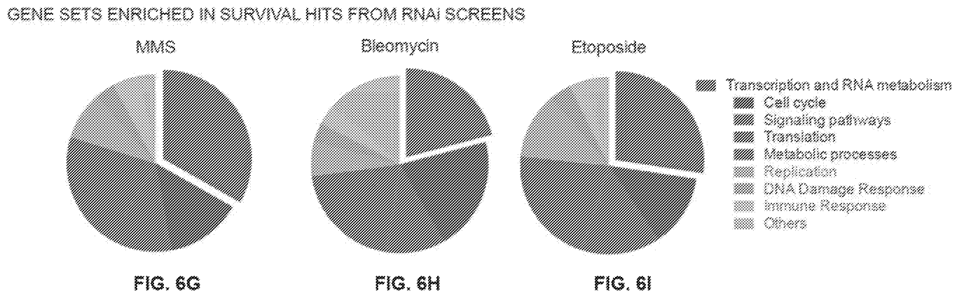

The Genomics of Drug Sensitivity in Cancer database was probed. The IC₅₀ of drugs inducing transcription and replication blocks in EWS-FLI1 associated cancers was nearly 5-fold lower compared to a pan-cancer dataset (FIGS. 5A and 5B). In order to identify pathways that contribute to the chemosensitivity in EwS, gene expression was analyzed over time following etoposide exposure in EwS and control cell lines (FIG. 6A). FIG. 6A is a heatmap of basal gene expression profile in control and EwS cell lines after hierarchical clustering. Gene set enrichment analysis of the differences in gene expression profile between EwS and control cell lines under basal conditions (no damage) extracted the expected EwS pathways and additionally, defects in replication, transcription and repair pathways (FIGS. 6B-6I). FIG. 6B is a table capturing the top enriched pathways from Gene Set Enrichment Analysis (GSEA) of the differences between EwS and control cell lines. FIGS. 6C-6F are the relevant GSEA signature plots. Apart from the expected enrichment of EWS-FLI1 driven genes and pediatric cancer signature, we found differential upregulation of replication stress, BRCA1-mutation driven network and altered transcription regulation pathways in EwS. FIGS. 6G-6I are illustrations of a cross-screen pathway comparison of top survival hits from RNAi screens in Drosophila Kc167 cells exposed to MMS, bleomycin or etoposide. Nearly a third of the top 5% hits in each screen were enriched for genes involved in transcription and RNA metabolism highlighting the importance of this pathway in DNA damage survival.

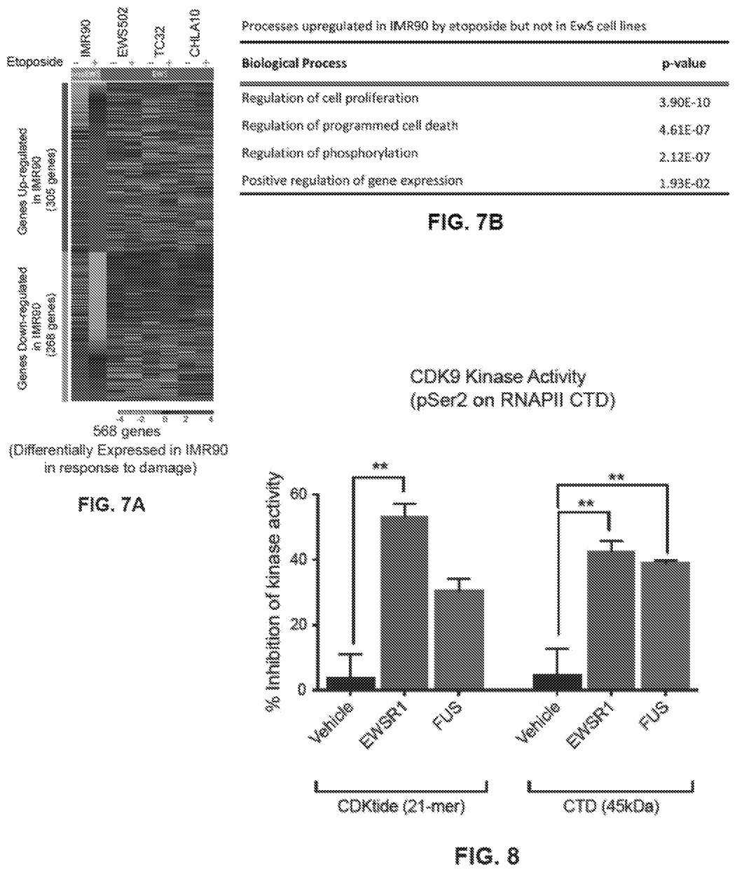

The differences in transcriptional response to damage was analyzed (FIGS. 7A and 7B, and Table 2). FIG. 7A is a heatmap of genes that are at least 2-fold altered in control (IMR90) cells in response to damage but not in the EwS cells (sorted by gene expression in IMR90). The selected genes were classified by the Gene Ontology annotation of biological processes (P<0.05) and the most significantly enriched pathways are listed in the tables of FIG. 7B.

Table 2 presents the damage-induced changes in gene expression between IMR90 and EwS cell lines. Differential expression of genes with etoposide treatment (6 hours) over untreated was generated using DESeq software and a minimum of two-fold change in gene expression was used as cut-off. Table 2 provides a summary of number of genes that were either similarly expressed or not similarly expressed between IMR90 and EwS.

TABLE 2

| Comparison of Damage-induced expression | |
|---|---|
| changes in IMR90 versus EwS | Number of genes |
| Genes common to IMR90 and EwS | 29 |
| Genes that respond to damage in IMR90 but | 568 |
| do not alter in a similar pattern in EwS | |
| Genes that respond to damage in EwS but | 133 |
| do not alter in a similar pattern in IMR90 | |

Table 3 provides a list of genes that were similarly altered by gene expression in response to damage between IMR90 and EwS.

TABLE 3
Log₂ [Etoposide_6 hr_treated/Untreated]

| GENE | IMR90 | TC32 | EWS502 | CHLA10 |
|---|---|---|---|---|
| HIST1H2BF | 3.252 | 1.36 | −0.85 | 5.158 |
| MT1F | 2.585 | 2.76 | 2.305 | 6.229 |
| HIST1H4E | 2.083 | 1.01 | 0.511 | 3.895 |
| MTUS2 | 1.668 | −1.861 | 1.627 | 1.252 |
| MIR210HG | −1.002 | −1.262 | −3.543 | −1.768 |
| STON1 | −1.026 | −1.525 | −2.54 | −1.403 |
| LOC284276 | −1.055 | — | −2.965 | −0.918 |
| CHL1 | −1.158 | — | −1.695 | −1.399 |
| MERTK | −1.18 | −0.762 | −1.836 | −1.016 |
| PIK3IP1 | −1.273 | −1.559 | 0.198 | −1.533 |
| ANKRD6 | −1.28 | −0.142 | −1.474 | −1.05 |
| SOCS1 | −1.412 | −1.358 | −1.455 | −0.105 |
| EYA4 | −1.426 | −0.197 | −2.314 | −1.112 |
| SNORA14B | −1.502 | −1.525 | −0.043 | −3.021 |
| CCDC89 | −1.524 | 0.934 | −1.28 | −1.958 |
| PIF1 | −1.613 | 0.34 | −1.42 | −1.635 |
| FAM65C | −2.017 | −1.114 | −1.855 | −1.193 |
| ABCG1 | −2.055 | −1.444 | −1.28 | −1.858 |
| RHPN1 | −2.502 | −1.09 | −1.206 | −0.401 |
| LOC143666 | −2.69 | −1.497 | −1.42 | −0.231 |
| CHST6 | −2.725 | −0.455 | −1.218 | −1.333 |
| NDP | −3.087 | −2.256 | −2.783 | −0.886 |
| TMEM100 | −4.087 | −1.322 | −2.599 | −0.887 |
| ASB11 | — | −1.822 | −0.588 | −1.417 |
| HOXD12 | — | −2.59 | −1.039 | −0.878 |
| MIR4664 | — | −2.424 | −2.28 | 0.422 |
| SYK | — | — | −1.206 | −1.312 |
| CRYBB2 | — | — | 2.689 | 1.65 |
| MT1G | — | −0.766 | — | — |

Table 4 provides a list of genes that were transcriptionally altered in response to damage exclusively in EwS cells. The median fold change from all EwS cell lines was used for evaluation.

TABLE 4
Log₂ [Etoposide_6 hr_treated/Untreated]

| GENE | IMR90 | TC32 | EWS502 | CHLA10 |
|---|---|---|---|---|
| PLCXD3 | 4.726 | −1.268 | 2.305 | −1.051 |
| LOC80054 | 2.89 | −3.218 | −2.553 | — |
| BEGAIN | 2.22 | −1.655 | −2.017 | 0.252 |
| CCL20 | 1.82 | 2.934 | −1.114 | −3.333 |
| KAL1 | 1.53 | — | −3.155 | −1.249 |
| VWA3A | 1.404 | 0.349 | −1.503 | −1.155 |
| MGP | 1.187 | −1.272 | −0.572 | −1.463 |
| CXCL14 | 1.083 | −1.027 | −3.28 | 1.711 |
| CYP24A1 | 1.083 | −1.088 | −1.503 | −0.246 |
| MT1E | 0.9 | 2.622 | 0.305 | 2.178 |
| ZNF792 | 0.873 | −1.388 | −1.21 | −1.014 |
| SPATA18 | 0.82 | 1.145 | −2.28 | −1.556 |
| ACTG2 | 0.669 | 0.076 | 1.374 | 1.111 |
| MYEOV | 0.623 | −1.917 | −1.391 | −0.324 |
| SLC30A1 | 0.6 | 3.415 | 1.107 | 1.507 |
| UBE2D4 | 0.59 | 0.917 | 1.095 | 1.551 |
| MT2A | 0.578 | 1.767 | 0.308 | 2.394 |
| PIK3CD | 0.565 | −1.247 | −1.461 | −0.641 |
| IL4I1 | 0.556 | — | −1.874 | −0.135 |
| CRYM | 0.535 | — | 0.305 | −1.163 |
| RBM24 | 0.53 | −0.066 | −2.632 | −3.556 |
| ICAM5 | 0.507 | −1.351 | −1.604 | −0.679 |
| STMN4 | 0.404 | −1.246 | −2.312 | −0.048 |
| ADAP1 | 0.358 | −1.557 | −1.036 | 0.752 |
| IGDCC4 | 0.282 | 1.612 | −2.259 | −1.28 |
| COL8A2 | 0.274 | −0.809 | −2.182 | −1.296 |
| C14orf49 | 0.269 | −0.569 | −1.104 | −1.534 |
| PRR19 | 0.252 | 0.083 | 1.183 | 1.478 |
| RPS28 | 0.251 | −2.646 | −2.378 | 0.079 |
| COL9A3 | 0.182 | −1.05 | −0.507 | −1.227 |
| LMCD1 | 0.153 | −1.416 | −1.425 | −0.34 |
| SDC1 | 0.114 | 0.973 | −2.54 | −1.485 |
| EGR1 | 0.109 | −1.315 | 1.385 | 2.2 |
| MIR4767 | 0.083 | 0.578 | 1.667 | 5.206 |
| GPR88 | 0.083 | −1.728 | — | 1 |
| AK7 | 0.083 | −0.164 | −1.28 | −1.514 |
| EPHX2 | 0.083 | 0.519 | −2.017 | −1.435 |
| LGI2 | 0.083 | 0.976 | −1.074 | −1.283 |
| RTN4RL1 | 0.083 | −1.766 | −1.632 | 0.091 |
| SERTM1 | 0.083 | −1.213 | −1.105 | −0.655 |
| SNORA36A | 0.083 | 0.934 | — | −2.748 |
| SYCP1 | 0.083 | −1.066 | −0.515 | −1.303 |
| SYNGR3 | 0.083 | −1.079 | −2.327 | −0.468 |
| TDRD9 | 0.083 | −2.233 | −1.396 | −0.4 |
| TMEM204 | 0.077 | −1.693 | 1.305 | 1.126 |
| FKRP | 0.06 | 0.088 | 1.008 | 1.081 |
| ZEB1-AS1 | 0.054 | 0.319 | −1.495 | −1.066 |
| BRPF3 | 0.052 | 0.333 | 1.033 | 1.162 |
| HNMT | 0.047 | −1.146 | −0.538 | −1.72 |
| FAP | 0.042 | −1.066 | −0.66 | −1.365 |
| RPL9 | 0.032 | −0.313 | −1.333 | −1.222 |
| ADM | 0.015 | −1.394 | −1.695 | −0.279 |
| ATP8B1 | 0.012 | −1.175 | −1.025 | −0.594 |
| GIPR | 0.012 | 0.519 | −2.943 | −1.717 |
| TMC6 | 0.012 | −1.596 | −1.435 | −0.129 |
| LOC613037 | 0.006 | 1.006 | 0.081 | 1.549 |
| MMP17 | −0.013 | −1.174 | −1.005 | 0.325 |
| CCDC80 | −0.023 | −1.091 | −1.078 | −0.718 |
| NPIPL3 | −0.043 | 1.182 | 0.066 | 1.851 |
| CLU | −0.043 | 1.177 | 0.116 | 1.023 |
| SCARNA27 | −0.069 | −1.525 | −2.017 | −2.655 |
| CCDC136 | −0.082 | −0.94 | −1.468 | −1.329 |
| SCARNA15 | −0.087 | — | 2.305 | −2.471 |
| PADI2 | −0.11 | 1.3 | −0.881 | 1.075 |
| RAPGEF5 | −0.11 | −1.507 | −2.017 | −1.713 |
| CRYAB | −0.131 | 1.963 | −0.335 | 1.788 |
| ORAI3 | −0.136 | −1.241 | −1.314 | −0.149 |
| ATHL1 | −0.228 | −0.282 | −1.16 | −1.108 |
| TST | −0.23 | −1.138 | −1.02 | −0.399 |
| TMEM171 | −0.232 | −2.001 | −1.695 | 0.193 |
| ABCA1 | −0.249 | 1.381 | −1.57 | −1.819 |
| EMID1 | −0.258 | −2.273 | −1.155 | 0.252 |
| FBLN7 | −0.264 | −2.772 | — | −0.312 |
| C9orf172 | −0.284 | 0.326 | 1.128 | 1.714 |
| MEPCE | −0.287 | 0.139 | 1.098 | 1.138 |
| PGM5 | −0.306 | −1.48 | −1.396 | −0.669 |
| RASD2 | −0.317 | −1.186 | −1.727 | −0.482 |
| RPS27 | −0.321 | −0.481 | 2.627 | 1.469 |
| C1QL2 | −0.332 | −1.066 | 2.527 | 1.125 |
| CXCL10 | −0.332 | — | −1.122 | −1.013 |
| DCX | −0.332 | 1.197 | −1.068 | −1.748 |
| LRRC4 | −0.358 | 1.422 | 1.372 | 1.805 |
| SLC39A10 | −0.36 | −1.151 | −0.717 | −1.109 |
| LOC221442 | −0.382 | 1.037 | 1.262 | −1.238 |
| CXCL12 | −0.401 | − | −1.104 | −1.112 |
| ASGR1 | −0.403 | −1.393 | −0.813 | −1.569 |
| RASSF2 | −0.446 | −0.554 | −1.795 | −1.033 |
| PPARA | −0.465 | −1.857 | −1.29 | −0.53 |
| PDGFRL | −0.476 | 1.741 | −1.532 | −1.261 |
| CPS1 | −0.482 | 0.324 | −1.469 | −1.181 |
| PKNOX2 | −0.497 | — | −1.381 | 0.158 |
| ODF3B | −0.502 | −1.721 | −1.759 | 0.444 |
| UNC93B1 | −0.514 | −1.469 | −0.406 | −1.029 |
| PTGIS | −0.561 | — | −1.352 | −1.381 |
| GSDMB | −0.568 | 0.08 | −1.058 | −1.4 |
| SECTM1 | −0.572 | −1.523 | −3.905 | 0.339 |
| NEAT1 | −0.603 | 0.038 | −1.655 | −2.145 |
| C14orf39 | −0.609 | 0.419 | −1.671 | −1.353 |
| N4BP2L1 | −0.628 | −2.09 | −1.979 | −1.177 |
| TXNIP | −0.681 | −1.442 | −0.517 | −1.463 |
| HIST2H2BF | −0.693 | 1.197 | −0.371 | 2.609 |
| AIM1 | −0.705 | 0.493 | −2.117 | −1.117 |
| LOC255512 | −0.733 | −2.254 | −1.162 | 0.252 |
| DENND2A | −0.743 | −1.206 | −1.561 | −0.818 |
| VCAM1 | −0.751 | — | −1.131 | −1.226 |
| PLEKHH2 | −0.752 | −0.036 | −1.606 | −1.502 |
| FBXO32 | −0.765 | −1.064 | −1.022 | −1.057 |
| TNFRSF4 | −0.765 | — | −1.604 | −1.546 |
| NFATC4 | −0.781 | −0.405 | −2.322 | −1.579 |
| TMEM200B | −0.797 | −1.538 | — | 0.837 |
| PDGFRB | −0.812 | −1.041 | −1.714 | −0.528 |
| RASSF4 | −0.825 | −1.066 | −2.052 | −0.773 |
| MIAT | −0.829 | 0.497 | −1.163 | −1.358 |
| ACSS1 | −0.832 | −2.304 | −1.298 | −0.949 |
| RNF112 | −0.836 | −1.388 | — | −2.176 |
| SAMD13 | −0.85 | −0.568 | −1.138 | −1.376 |
| KCNK1 | −0.891 | — | — | −1.2 |
| TRIL | −0.917 | −2.503 | −3.269 | −0.668 |
| POLR2J2 | −0.935 | −2.008 | −2.482 | 0.367 |
| MIR4461 | −1.2 | 3.229 | −1.649 | 2.474 |
| MTRNR2L2 | −1.239 | 1.246 | −0.28 | 2.791 |
| MESP2 | −2.033 | 1.004 | −0.671 | 1.522 |
| HIST1H2AB | −2.2 | 3.072 | −0.28 | 3.059 |
| HSP90AA6P | −2.976 | 1.344 | 0.166 | 2.273 |
| APOL4 | — | 0.934 | −1.251 | −1.169 |
| CCNA1 | — | 0.461 | −1.336 | −1.695 |
| CLEC2L | — | −1.885 | −1.695 | 1.351 |
| ISLR2 | — | −2.057 | −1.695 | 0.048 |
| KCNJ4 | — | −1.718 | −1.695 | −0.011 |
| LOC100505483 | — | 0.934 | −1.503 | −1.29 |
| LOC100507351 | — | 1.671 | −1.049 | −1.06 |
| LRRC10B | — | −1.933 | −1.471 | 0.376 |
| TEKT2 | — | −0.829 | −1.165 | −1.779 |

The most significant difference was noted in the set of genes that responded to damage in IMR90 but displayed little to no response in EwS. The genes that were transcriptionally altered after etoposide exposure in control cells (IMR90) were mapped. From this dataset, a subset of genes were selected that did not display the same trend in response to etoposide in EwS (568 genes, FIGS. 7A and 7B, Table 5).

Table 5 provides a list of genes that are at least two-fold altered upon etoposide treatment in IMR90 but not in EwS cell lines. The criterion for evaluation of EwS cells was as follows: genes that were upregulated atleast 2-fold in IMR90 but less than 0-fold (no change or downregulated) in EwS and vice versa.

TABLE 5
Log₂ [Etoposide_6 hr_treated/Untreated]

| GENE | IMR90 | TC32 | EWS502 | CHLA10 |
|---|---|---|---|---|
| LCE1F | 8.1 | 1.2 | 0.3 | 0.2 |
| MIR3189 | 7.6 | 0.5 | 0.3 | 0.2 |
| SNORD4B | 6.3 | 1.9 | 1.7 | −0.9 |
| SNORD116-26 | 5.7 | −2.6 | 0.4 | 0.5 |
| MIR450B | 5.3 | 0.4 | 0.2 | 0.2 |
| MIR23A | 5.3 | −2.4 | 0.3 | 0.5 |
| ADM2 | 5.0 | 0.6 | −0.5 | −0.8 |
| MIR21 | 4.9 | 1.1 | −2.7 | 0.2 |
| MIR4519 | 4.8 | −0.9 | −2.8 | 2.3 |
| NPPC | 4.5 | 1.5 | 0.4 | 0.3 |
| SNORA38 | 4.4 | −5.3 | 2.4 | 0.4 |
| GDF15 | 4.0 | 2.3 | 1.0 | 0.1 |
| PTGS2 | 3.9 | 1.4 | 0.4 | 0.3 |
| ULBP1 | 3.9 | 0.6 | −0.1 | −0.1 |
| SNORD12B | 3.7 | 1.0 | 0.4 | 0.1 |
| XIRP1 | 3.6 | 0.2 | 1.1 | 0.1 |
| ANGPTL4 | 3.5 | 0.7 | −0.2 | 1.7 |
| ASNS | 3.5 | 0.8 | 0.2 | −0.6 |
| CSF2 | 3.2 | −2.6 | 0.2 | 0.1 |
| RRAD | 3.2 | 1.0 | 1.1 | −1.0 |
| LCE2C | 3.2 | 1.2 | 0.3 | 0.2 |
| TMEM156 | 3.0 | 0.4 | −4.0 | −3.8 |
| INHBE | 2.9 | 2.5 | 0.3 | −1.9 |
| ESM1 | 2.9 | −3.8 | 0.6 | 0.1 |
| TRIB3 | 2.9 | −0.3 | 0.1 | −0.1 |
| SLC6A9 | 2.9 | 0.2 | 0.3 | 0.2 |
| PTHLH | 2.8 | 1.2 | 1.1 | −0.5 |
| PCK2 | 2.8 | 0.3 | −0.2 | −0.8 |
| KRT34 | 2.8 | 1.0 | −4.0 | 4.3 |
| SLC7A5 | 2.8 | 0.3 | 0.1 | 0.5 |
| MIR3117 | 2.7 | 0.4 | −2.7 | −3.8 |
| DDIT4 | 2.7 | 0.4 | −1.7 | −0.9 |
| TAGLN3 | 2.7 | −0.3 | −1.4 | 0.2 |
| PSAT1 | 2.7 | 0.4 | 0.1 | −0.5 |
| KRTAP4-12 | 2.6 | −3.9 | −2.9 | 0.1 |
| SNORD95 | 2.6 | 0.4 | 1.3 | −0.2 |
| C4orf32 | 2.5 | 0.6 | −0.7 | 0.3 |
| VWCE | 2.5 | 0.3 | −0.5 | 0.2 |
| VLDLR | 2.5 | 1.5 | 0.3 | −1.0 |
| KCNG1 | 2.5 | 0.8 | −0.6 | 1.0 |
| LRFN5 | 2.4 | 0.1 | −0.2 | 0.0 |
| ATF3 | 2.4 | 1.1 | 0.7 | 0.0 |
| SNORD116-1 | 2.3 | −2.0 | 0.0 | −1.1 |
| CLDN1 | 2.3 | 1.4 | −0.1 | 0.0 |
| C10orf47 | 2.3 | 0.1 | −0.1 | 0.6 |
| UNC5B | 2.3 | 0.0 | 0.8 | 0.2 |
| SERPINB2 | 2.3 | 0.0 | 1.5 | 0.8 |
| HBEGF | 2.2 | 0.3 | 0.4 | 0.6 |
| SESN2 | 2.2 | 1.2 | 0.1 | −0.2 |
| CTH | 2.2 | 0.4 | 0.1 | 0.0 |
| TRIML2 | 2.2 | −2.6 | 0.1 | −2.6 |
| TCTEX1D4 | 2.2 | 0.7 | −0.9 | −1.0 |
| KRTAP2-4 | 2.2 | 0.9 | 0.2 | 0.1 |
| LOC284023 | 2.2 | −0.5 | 0.8 | 0.3 |
| HIST1H4E | 2.2 | 1.2 | 0.6 | 4.6 |
| SLC4A5 | 2.2 | 0.3 | 0.0 | −1.0 |
| MIR27A | 2.2 | 0.1 | 0.2 | 0.2 |
| SNORD116-12 | 2.2 | 4.0 | −1.2 | 0.4 |
| SNORD116-16 | 2.2 | 1.1 | −4.9 | 0.4 |
| MIR3613 | 2.1 | −2.6 | 2.2 | 4.6 |
| MURC | 2.1 | −0.1 | −0.5 | 0.1 |
| C6orf105 | 2.1 | 4.0 | 0.1 | −2.5 |
| MTHFD2 | 2.1 | 0.3 | 0.3 | −0.5 |
| PRDM1 | 2.1 | 1.3 | 0.3 | −0.6 |
| BTG2 | 2.0 | 1.2 | 0.3 | 0.1 |
| SNORA71C | 2.0 | 1.3 | 0.1 | −0.1 |
| LAMC2 | 2.0 | 2.1 | −1.3 | −3.0 |
| ACER2 | 2.0 | 1.0 | 0.7 | 0.5 |
| EDN1 | 2.0 | 0.3 | −0.6 | 2.0 |
| HCG23 | 2.0 | 6.2 | −0.4 | 1.1 |
| RPL7 | 2.0 | −2.3 | 0.1 | −0.5 |
| ANKRD1 | 2.0 | −1.5 | 0.3 | −0.5 |
| TSPAN2 | 2.0 | −0.5 | −0.3 | −0.2 |
| SLC1A4 | 2.0 | −0.7 | 0.0 | 0.7 |
| NGF | 1.9 | 3.8 | 0.4 | −0.7 |
| THSD1 | 1.9 | −0.4 | 0.5 | 0.5 |
| LOC375295 | 1.9 | −1.1 | 0.5 | −0.5 |
| CD274 | 1.9 | 4.2 | 0.0 | −3.4 |
| PSPH | 1.9 | 0.1 | −0.1 | −0.7 |
| AMZ1 | 1.9 | −5.9 | −4.4 | −0.3 |
| FCRLA | 1.9 | 4.0 | −2.6 | 0.1 |
| MIR4467 | 1.9 | 1.6 | −0.5 | −1.2 |
| TNFRSF10C | 1.9 | 2.1 | 0.8 | −0.4 |
| CDKN1A | 1.9 | 2.7 | −0.1 | 0.0 |
| NSFP1 | 1.9 | 2.0 | 0.1 | −5.1 |
| ITPKA | 1.9 | −0.9 | 0.0 | 0.5 |
| TSLP | 1.9 | −2.6 | 2.6 | 1.3 |
| F3 | 1.9 | 0.4 | 0.3 | 0.0 |
| BBC3 | 1.8 | 0.4 | 0.1 | −0.1 |
| ADAMTS6 | 1.8 | 0.1 | −2.0 | −1.0 |
| AMIGO2 | 1.8 | −0.9 | 0.6 | −0.9 |
| NRG1 | 1.8 | 0.1 | −0.3 | 0.1 |
| UCN2 | 1.8 | 3.9 | −0.2 | 0.6 |
| SNORA39 | 1.8 | −1.7 | 0.6 | 0.5 |
| SLC7A11 | 1.8 | 0.7 | 0.5 | −0.5 |
| SNORD42A | 1.8 | 1.1 | 0.5 | −2.8 |
| MIR4677 | 1.8 | 6.4 | 1.2 | 3.3 |
| SNORD46 | 1.7 | −1.6 | −0.4 | −0.3 |
| MIR181A2HG | 1.7 | 0.9 | −0.2 | −0.2 |
| PLEK2 | 1.7 | 1.7 | 3.3 | −0.4 |
| HAS2 | 1.7 | −1.1 | 3.3 | 0.1 |
| IL11 | 1.7 | 1.5 | −1.9 | −0.7 |
| VEGFA | 1.7 | 0.7 | −0.4 | −0.3 |
| SIK1 | 1.7 | −0.4 | 0.5 | 0.6 |
| SPHK1 | 1.7 | −0.9 | −1.2 | 1.7 |
| TNFRSF10A | 1.7 | 0.1 | −2.5 | 0.0 |
| CRIP1 | 1.7 | −0.2 | −0.2 | 0.7 |
| PMAIP1 | 1.7 | 0.7 | 0.0 | −0.2 |
| TIMP3 | 1.7 | 0.8 | −0.1 | 0.3 |
| OSTN | 1.7 | −1.8 | 0.5 | 0.3 |
| IFLTD1 | 1.7 | −2.5 | −1.5 | 0.1 |
| OLFM2 | 1.7 | −0.7 | −0.3 | 0.4 |
| CYFIP2 | 1.6 | 0.5 | −0.4 | 0.4 |
| OSBPL6 | 1.6 | 0.1 | −0.3 | −0.1 |
| PLK3 | 1.6 | 1.2 | 0.1 | 0.8 |
| UPP1 | 1.6 | −1.0 | 0.1 | −0.1 |
| LOC283050 | 1.6 | 0.3 | −0.5 | −1.3 |
| SNORD63 | 1.6 | 1.0 | −0.4 | −0.5 |
| C5orf30 | 1.6 | −0.3 | 0.0 | −0.3 |
| C3orf52 | 1.6 | 0.1 | 0.0 | −0.5 |
| CITED4 | 1.6 | 0.4 | −0.5 | 2.0 |
| ERRFI1 | 1.6 | −0.6 | 0.2 | −0.3 |
| DIRAS3 | 1.6 | 0.8 | 0.5 | 0.8 |
| NRXN3 | 1.6 | −4.1 | 3.3 | 3.2 |
| ALPL | 1.5 | 0.6 | 0.0 | 1.3 |
| BHLHE40 | 1.5 | 0.4 | −1.3 | 0.2 |
| SDPR | 1.5 | −0.2 | −1.2 | −1.3 |
| PDGFA | 1.5 | −1.2 | 3.2 | 4.0 |
| RELB | 1.5 | −0.6 | 0.2 | 1.5 |
| KCTD4 | 1.5 | 0.3 | −2.6 | −2.4 |
| SNORA68 | 1.5 | 1.3 | −1.5 | 0.5 |
| IFNE | 1.5 | 0.4 | 3.3 | −6.3 |
| GDNF | 1.5 | −0.4 | −0.2 | 2.6 |
| MIR409 | 1.5 | 0.4 | 0.2 | 3.3 |
| DDIT4L | 1.5 | −0.6 | −1.2 | −0.9 |
| SNORD116-28 | 1.5 | 0.5 | 1.7 | −1.9 |
| TNFAIP8L3 | 1.5 | 0.3 | 2.6 | 0.1 |
| MAMDC4 | 1.5 | 1.4 | −0.3 | 0.0 |
| FDXR | 1.5 | 1.1 | −0.1 | 0.1 |
| CEBPG | 1.5 | 0.1 | 0.1 | −0.5 |
| KCNH1 | 1.5 | −4.1 | 0.1 | −1.1 |
| SLC7A1 | 1.5 | 0.3 | 0.1 | −0.1 |
| LIF | 1.5 | 2.7 | −1.2 | −1.7 |
| ENCI | 1.5 | 1.9 | 0.6 | 0.2 |
| IL13RA2 | 1.5 | 3.9 | 0.1 | −1.1 |
| PHGDH | 1.5 | 0.3 | 0.1 | 0.1 |
| CBS | 1.5 | −0.2 | −0.5 | −0.5 |
| SYT16 | 1.5 | 0.2 | −0.2 | 0.2 |
| ZFHX2 | 1.4 | −0.1 | 0.2 | 1.2 |
| TUBB2B | 1.4 | 0.4 | −0.6 | −1.0 |
| RPLP0P2 | 1.4 | −0.5 | 0.2 | 0.9 |
| ALDH1L2 | 1.4 | 1.0 | −0.3 | −0.8 |
| ADAM19 | 1.4 | −0.8 | −0.2 | 0.0 |
| SCARNA8 | 1.4 | 1.6 | 0.1 | −4.0 |
| SNORD20 | 1.4 | 4.1 | −0.4 | 1.1 |
| ELFN1 | 1.4 | −0.4 | −3.8 | 3.0 |
| MIR24-2 | 1.4 | −2.4 | 0.3 | 0.2 |
| ITGB3 | 1.4 | −1.7 | 0.3 | −0.2 |
| MIR1914 | 1.4 | 0.6 | 0.1 | 0.7 |
| POU2F2 | 1.4 | 1.1 | −0.1 | 0.2 |
| MIR4258 | 1.4 | −0.4 | 0.2 | −0.1 |
| ERN1 | 1.4 | 0.6 | 0.4 | 0.5 |
| PTX3 | 1.4 | −4.6 | 0.1 | −0.2 |
| COL7A1 | 1.3 | −0.5 | −0.6 | −1.7 |
| TSC22D3 | 1.3 | 0.3 | 0.4 | −0.5 |
| RAP1GAP2 | 1.3 | −0.8 | 1.3 | −3.5 |
| NPAS1 | 1.3 | 0.5 | 0.1 | 0.7 |
| SYBU | 1.3 | 0.3 | 0.9 | −0.3 |
| EFHD1 | 1.3 | 2.5 | −0.6 | 0.5 |
| PCDHGC5 | 1.3 | 0.8 | −0.4 | −0.4 |
| C9orf150 | 1.3 | −1.8 | −2.9 | −1.4 |
| ZNF643 | 1.3 | 0.3 | 0.7 | 0.5 |
| CARD9 | 1.3 | −0.1 | 1.2 | 0.1 |
| COL5A3 | 1.3 | −0.9 | 1.8 | −2.4 |
| PDF | 1.3 | −1.4 | 0.2 | 2.0 |
| HAS2-AS1 | 1.3 | 0.3 | 3.3 | 0.1 |
| SNORD34 | 1.3 | 1.3 | 0.2 | 0.5 |
| SLC1A5 | 1.3 | 0.0 | 0.3 | 0.7 |
| STYK1 | 1.3 | 1.2 | 0.3 | 1.5 |
| PSTPIP2 | 1.3 | 0.2 | −0.2 | 0.1 |
| SEMA7A | 1.3 | −1.2 | −0.5 | 0.2 |
| PTPRB | 1.3 | 0.5 | −0.4 | −0.2 |
| CRYGS | 1.3 | 0.7 | 0.1 | 0.4 |
| SNORD55 | 1.3 | −0.3 | 0.1 | 0.9 |
| 42432.0 | 1.3 | −0.6 | 0.6 | 0.1 |
| RNF19B | 1.3 | 0.9 | 0.3 | 0.6 |
| C14orf82 | 1.3 | −0.1 | 0.9 | 0.4 |
| WDR63 | 1.3 | 3.3 | 0.8 | 0.3 |
| HTR7 | 1.3 | −2.5 | −3.8 | 2.9 |
| LCE2B | 1.3 | 1.2 | 0.3 | 0.2 |
| ANXA3 | 1.3 | 0.4 | 0.1 | −1.9 |
| ICAM1 | 1.3 | −0.4 | −1.1 | 0.5 |
| RHBDF2 | 1.3 | −0.2 | −0.2 | 0.9 |
| GADD45A | 1.3 | 1.0 | 0.1 | 0.0 |
| IL12A | 1.2 | 0.1 | −0.7 | 0.7 |
| ZNF311 | 1.2 | 1.1 | −0.4 | −3.0 |
| MDM2 | 1.2 | 1.2 | 0.4 | −0.2 |
| TMEM154 | 1.2 | 1.3 | −5.5 | −0.3 |
| JHDM1D | 1.2 | −0.2 | −0.4 | −0.2 |
| SH3BP5L | 1.2 | −0.1 | 0.2 | 0.2 |
| BPGM | 1.2 | 0.6 | 0.5 | 0.0 |
| NOX4 | 1.2 | 0.2 | 0.6 | 0.9 |
| IL6 | 1.2 | −2.6 | 2.6 | 0.1 |
| S1PR1 | 1.2 | −2.5 | −1.4 | −2.5 |
| NUPR1 | 1.2 | 2.3 | 0.4 | −0.8 |
| TBC1D2 | 1.2 | −0.2 | 0.7 | 0.7 |
| CHST2 | 1.2 | 1.8 | 0.7 | 2.6 |
| NXPH4 | 1.2 | 0.9 | −0.1 | 1.4 |
| MIR369 | 1.2 | −3.3 | 0.3 | 0.3 |
| MIR3677 | 1.2 | −1.5 | 4.6 | −2.8 |
| MSC | 1.2 | 0.9 | −0.3 | −0.1 |
| TNFSF4 | 1.2 | 0.6 | 0.5 | 0.1 |
| PMEPA1 | 1.2 | −0.9 | 0.7 | 0.4 |
| SLC3A2 | 1.2 | 0.2 | 0.2 | 0.2 |
| ADRB2 | 1.2 | 0.3 | 0.6 | 0.1 |
| FRS3 | 1.2 | 0.1 | −0.4 | 0.2 |
| SNORD99 | 1.2 | 1.0 | 1.4 | −0.5 |
| FZD8 | 1.2 | 0.6 | 0.0 | 1.2 |
| PRSS3 | 1.2 | 0.5 | 0.1 | −0.8 |
| SNORD114-26 | 1.2 | 0.6 | 0.3 | 0.2 |
| HIVEP3 | 1.2 | 0.5 | 1.2 | 1.6 |
| PKP1 | 1.2 | 1.2 | 0.0 | 0.1 |
| NCF2 | 1.2 | 0.4 | 2.2 | −2.4 |
| FAS | 1.2 | 0.0 | −0.6 | 0.0 |
| SNORD116-23 | 1.2 | −1.5 | 0.6 | 0.5 |
| MIR3605 | 1.2 | 1.0 | 1.0 | −1.0 |
| PGAM2 | 1.2 | −0.7 | 0.8 | 0.1 |
| C6orf132 | 1.2 | −0.5 | −0.4 | −5.1 |
| CBLB | 1.2 | −0.1 | −0.1 | 0.0 |
| SPSB1 | 1.2 | −0.1 | 0.5 | 0.9 |
| CARS | 1.1 | 0.2 | 0.4 | −0.4 |
| LOC554202 | 1.1 | −2.6 | 3.3 | −0.3 |
| MAST4 | 1.1 | 0.0 | −0.4 | −0.1 |
| RUSC2 | 1.1 | 0.1 | −0.1 | 0.6 |
| SNORA69 | 1.1 | 1.0 | 0.6 | −0.4 |
| ADRA1D | 1.1 | 0.1 | −0.7 | 0.8 |
| SULF2 | 1.1 | 0.4 | 0.3 | −0.6 |
| DUSP5 | 1.1 | −0.9 | 0.9 | 0.1 |
| SERPINE1 | 1.1 | 0.7 | 0.7 | 0.2 |
| GAL | 1.1 | 0.5 | −0.3 | 1.0 |
| TUBE1 | 1.1 | 0.7 | 0.2 | −0.4 |
| DSP | 1.1 | 0.6 | −1.2 | 0.5 |
| MIR4737 | 1.1 | 0.2 | 1.0 | −1.0 |
| SLC9A1 | 1.1 | 0.5 | 0.8 | 1.5 |
| ZFPM1 | 1.1 | 0.5 | −0.2 | 2.9 |
| SNORA71B | 1.1 | −0.9 | 1.5 | −0.7 |
| KLF2 | 1.1 | −0.3 | 0.3 | 2.7 |
| EXTL1 | 1.1 | −0.7 | −1.8 | −1.0 |
| GEMC1 | 1.1 | −0.3 | −0.5 | −0.4 |
| C21orf122 | 1.1 | −0.5 | −0.5 | −0.2 |
| IER3 | 1.1 | −0.5 | −0.4 | 0.0 |
| PNP | 1.1 | 0.1 | −0.1 | 0.0 |
| PIDD | 1.1 | 0.1 | 0.0 | 0.5 |
| GALNTL4 | 1.1 | 4.0 | 0.1 | −4.6 |
| FGF2 | 1.1 | 0.2 | 0.1 | 0.1 |
| MARS | 1.1 | 0.5 | 0.2 | −0.1 |
| C3orf67 | 1.1 | 0.9 | −0.8 | −0.3 |
| HIST1H2AE | 1.1 | 0.8 | 0.0 | 1.5 |
| TFEB | 1.1 | −0.8 | −0.9 | −0.1 |
| SLC35F2 | 1.1 | 0.5 | 0.1 | 0.4 |
| GARS | 1.1 | 0.6 | 0.3 | 0.2 |
| ULBP2 | 1.1 | 8.0 | 0.5 | −0.2 |
| PKIA | 1.1 | −0.1 | 0.0 | −0.2 |
| LOC100287314 | 1.1 | −2.5 | 0.1 | 1.8 |
| NLRP10 | 1.1 | −2.6 | 0.1 | 0.1 |
| PIP5KL1 | 1.1 | −0.1 | 0.4 | 1.4 |
| KBTBD8 | 1.1 | 0.7 | 1.3 | 0.7 |
| ZNF600 | 1.1 | 0.3 | −0.3 | 0.0 |
| LOC148189 | 1.1 | −0.4 | 0.3 | 0.4 |
| NLRP1 | 1.1 | 0.1 | 0.0 | −0.4 |
| SERTAD1 | 1.1 | −0.1 | 0.8 | 0.8 |
| RGS20 | 1.1 | 3.0 | 0.4 | 1.3 |
| RPL23AP64 | 1.1 | 0.2 | 0.9 | −0.6 |
| C15orf37 | 1.1 | 0.0 | 0.9 | 0.8 |
| TARS | 1.1 | 0.1 | 0.0 | −0.5 |
| BAK1 | 1.1 | 0.5 | 0.2 | 0.5 |
| XYLT1 | 1.1 | 0.8 | 0.3 | 0.5 |
| MAFF | 1.0 | −0.9 | −0.3 | 0.4 |
| CHIC2 | 1.0 | −0.5 | 0.2 | −0.2 |
| LGALS9 | 1.0 | −0.1 | 0.2 | 0.0 |
| RGS4 | 1.0 | −1.4 | 0.5 | 0.4 |
| CCDC3 | 1.0 | 0.9 | 1.0 | 0.2 |
| CCNJL | 1.0 | −0.1 | 0.1 | 0.1 |
| SGK1 | 1.0 | −0.1 | 0.3 | 0.4 |
| SNORD124 | 1.0 | 0.8 | −1.7 | 0.8 |
| SLC4A11 | 1.0 | −0.1 | −0.9 | 1.0 |
| FLJ46906 | 1.0 | 0.4 | −0.5 | −1.7 |
| LCE2A | 1.0 | 4.4 | 0.3 | 0.2 |
| C12orf5 | 1.0 | 1.8 | 0.3 | −0.2 |
| BATF3 | 1.0 | 0.1 | −1.6 | 0.4 |
| SEL1L3 | 1.0 | 0.3 | −0.4 | −0.7 |
| TNFRSF10B | 1.0 | 0.3 | −0.2 | −0.8 |
| E2F7 | 1.0 | 0.7 | 0.7 | 0.6 |
| SNORD33 | 1.0 | 0.4 | −0.1 | 0.0 |
| FAM180A | 1.0 | 0.4 | 0.1 | 0.5 |
| EFNB2 | 1.0 | 0.9 | 0.3 | 0.3 |
| ZNF583 | 1.0 | 0.5 | 1.2 | 0.2 |
| SNORD100 | 1.0 | 0.0 | 1.7 | 0.4 |
| RHPN2 | 1.0 | 0.0 | −0.5 | −0.3 |
| PDLIM3 | 1.0 | 0.2 | −0.5 | −1.5 |
| PVR | 1.0 | 0.2 | 0.2 | 0.5 |
| PTGER4 | 1.0 | 1.6 | −0.4 | 0.3 |
| SARS | 1.0 | 0.6 | −0.1 | −0.1 |
| NEDD9 | 1.0 | −0.9 | 0.1 | 0.4 |
| MIR25 | −1.0 | −0.1 | −0.8 | −1.3 |
| DAAM2 | −1.0 | −0.3 | −0.8 | −0.4 |
| C12orf59 | −1.0 | 6.3 | 0.2 | 1.3 |
| PTCH1 | −1.0 | −0.5 | 0.0 | 0.0 |
| FOXF1 | −1.0 | −1.5 | 1.3 | −1.3 |
| KLHL13 | −1.0 | 0.1 | −0.3 | −0.4 |
| LIPE | −1.0 | 0.0 | −1.0 | 0.8 |
| LOC728743 | −1.0 | −0.4 | −0.1 | 0.2 |
| WEE1 | −1.0 | −0.2 | −0.2 | −0.3 |
| ANLN | −1.0 | 0.8 | −0.4 | −0.3 |
| INCENP | −1.0 | 0.7 | −0.4 | 0.0 |
| EVI2A | −1.0 | 4.1 | 0.1 | −2.5 |
| SAMD11 | −1.0 | −0.1 | 0.7 | 0.3 |
| SPATA6 | −1.0 | 0.1 | −0.7 | −0.2 |
| MRVI1 | −1.0 | −0.5 | −0.7 | −0.1 |
| NPTX1 | −1.0 | 1.3 | 0.3 | 1.0 |
| AURKB | −1.0 | 0.4 | −0.3 | −0.2 |
| CBR3 | −1.0 | −0.1 | −0.4 | −0.1 |
| HMSD | −1.0 | −0.2 | −0.9 | 0.0 |
| MID1 | −1.0 | 0.2 | 0.0 | 0.1 |
| FGL2 | −1.1 | 0.2 | −4.4 | 0.2 |
| CKAP2 | −1.1 | 0.5 | −0.1 | −0.1 |
| MAN1C1 | −1.1 | −0.6 | −0.8 | 0.5 |
| FBXO36 | −1.1 | 0.4 | −0.8 | −0.6 |
| NUSAP1 | −1.1 | 0.5 | −0.4 | −0.4 |
| PBK | −1.1 | 0.3 | −0.3 | −1.0 |
| TACC3 | −1.1 | 0.4 | −0.4 | −0.1 |
| CTTNBP2 | −1.1 | −0.2 | 0.1 | −0.2 |
| CCNE2 | −1.1 | 0.2 | 0.7 | 0.3 |
| DEPDC1 | −1.1 | 0.6 | −0.4 | −0.8 |
| BARD1 | −1.1 | −0.1 | 0.2 | −0.3 |
| CCNB2 | −1.1 | 0.2 | −0.3 | −0.6 |
| CIT | −1.1 | 0.6 | −0.5 | −0.3 |
| MEIS1 | −1.1 | 0.1 | −0.3 | −0.5 |
| MITF | −1.1 | 0.9 | 0.5 | 0.4 |
| RNF39 | −1.1 | −1.1 | 0.0 | 0.6 |
| TNFAIP8L1 | −1.1 | −0.1 | −1.4 | −0.6 |
| SNAP25 | −1.1 | 1.6 | 0.1 | 1.0 |
| TMPO | −1.1 | −0.1 | 0.0 | −0.3 |
| PRSS35 | −1.1 | −0.3 | −0.1 | −0.4 |
| VWA5A | −1.1 | −0.5 | −0.2 | 1.3 |
| CASC5 | −1.1 | 0.8 | −0.1 | −0.4 |
| HR | −1.1 | −0.4 | −0.9 | 0.0 |
| RNF122 | −1.1 | −0.3 | 0.1 | 0.5 |
| CCDC102B | −1.1 | −0.4 | −1.2 | −0.1 |
| MIR4461 | −1.1 | 3.3 | −1.5 | 2.9 |
| SLC29A2 | −1.1 | −0.2 | −0.6 | 0.2 |
| SSX2IP | −1.1 | 0.1 | −0.2 | −0.9 |
| AVPI1 | −1.1 | −0.5 | −0.1 | 0.6 |
| STIL | −1.1 | 0.8 | 0.0 | 0.0 |
| BTN3A1 | −1.1 | 0.0 | −0.4 | −0.6 |
| FAM72B | −1.1 | 0.9 | −0.5 | −0.5 |
| SLC2A12 | −1.1 | −0.1 | −0.3 | −0.9 |
| MAB21L2 | −1.1 | −2.5 | 0.0 | 0.1 |
| PIK3R3 | −1.1 | 1.1 | 0.4 | 0.2 |
| CDKN1C | −1.1 | −2.2 | −1.3 | −0.7 |
| MIR27B | −1.1 | −4.5 | −4.9 | 0.5 |
| CNKSR3 | −1.1 | −0.7 | 0.2 | 0.0 |
| GATS | −1.1 | −0.5 | −0.4 | 0.4 |
| CHRM2 | −1.1 | −1.0 | 0.1 | 0.4 |
| RPSA | −1.1 | −0.9 | 1.2 | −1.1 |
| AURKAPS1 | −1.1 | 0.7 | −0.8 | −0.7 |
| GAS2L3 | −1.1 | 0.9 | −1.1 | −0.2 |
| NCKAP5 | −1.1 | −0.1 | −0.5 | −0.5 |
| CDCA3 | −1.2 | 0.5 | −0.4 | −0.7 |
| SMC4 | −1.2 | 0.5 | 0.0 | −0.5 |
| SLFN11 | −1.2 | 0.1 | −0.1 | −0.4 |
| SH2B2 | −1.2 | 0.1 | −0.8 | 1.1 |
| KIF23 | −1.2 | 0.7 | −0.3 | −0.5 |
| KIAA1199 | −1.2 | 0.1 | 0.4 | 0.6 |
| IL6R | −1.2 | −0.1 | −0.9 | −0.7 |
| PKDCC | −1.2 | −0.2 | −0.7 | −0.4 |
| SPIRE2 | −1.2 | −0.5 | −0.1 | 0.1 |
| TRIM59 | −1.2 | 0.1 | −0.3 | −0.5 |
| ANKRD32 | −1.2 | 0.1 | 0.4 | −0.3 |
| HS6ST1 | −1.2 | 0.2 | 0.4 | 0.8 |
| RNU6ATAC | −1.2 | 0.8 | −0.3 | −0.4 |
| GTSE1 | −1.2 | 0.2 | −0.4 | −0.3 |
| SAP30 | −1.2 | −0.1 | −0.1 | −0.3 |
| RACGAP1 | −1.2 | 0.3 | −0.4 | −0.6 |
| GCNT4 | −1.2 | 0.1 | 0.0 | 0.0 |
| KIF15 | −1.2 | 0.5 | −0.1 | −0.6 |
| ZNF395 | −1.2 | −1.2 | −0.9 | −0.1 |
| SYTL2 | −1.2 | 0.4 | 1.5 | 0.8 |
| MIR614 | −1.2 | 0.4 | −2.8 | −2.7 |
| HLA-F-AS1 | −1.2 | −0.2 | −1.2 | −1.2 |
| LRRC20 | −1.2 | −0.2 | −0.5 | 0.4 |
| TCF21 | −1.2 | 0.2 | −2.8 | −2.6 |
| KIFC1 | −1.2 | 0.5 | −0.2 | −0.3 |
| LOC255480 | −1.2 | −0.8 | 0.6 | −0.4 |
| ARHGAP11B | −1.2 | 0.1 | −0.8 | −0.7 |
| BAHCC1 | −1.2 | −0.5 | −0.2 | 0.3 |
| CXXC5 | −1.2 | −1.3 | 0.2 | −0.6 |
| HIST1H2BJ | −1.2 | 0.6 | 0.3 | 0.9 |
| MEIS2 | −1.2 | −0.4 | −0.4 | −0.2 |
| PIK3IP1 | −1.2 | −1.5 | 0.5 | −1.6 |
| WDR76 | −1.2 | 0.2 | 0.1 | −0.2 |
| NUF2 | −1.2 | 0.5 | −0.5 | −1.2 |
| ARHGAP11A | −1.2 | 0.4 | −0.2 | −0.6 |
| DBF4 | −1.2 | 0.8 | −0.2 | −0.1 |
| E2F8 | −1.2 | 0.4 | 0.3 | 0.2 |
| ETV1 | −1.2 | −0.2 | −0.8 | 0.3 |
| FOXO4 | −1.2 | −0.3 | −0.2 | −0.4 |
| GSG2 | −1.2 | 0.6 | 0.3 | 0.5 |
| ORC1 | −1.2 | 0.0 | 0.6 | 0.5 |
| HMGB2 | −1.2 | 0.2 | −0.4 | −1.1 |
| NCAPG | −1.2 | 0.5 | −0.2 | −0.6 |
| PRRX2 | −1.2 | 0.0 | −1.2 | 1.0 |
| SMAD9 | −1.2 | −0.4 | 0.3 | −0.1 |
| NEURL1B | −1.3 | 0.3 | −1.0 | −0.1 |
| CDC25B | −1.3 | 0.4 | −0.6 | 0.2 |
| C20orf94 | −1.3 | 0.8 | 1.6 | 0.5 |
| VASH2 | −1.3 | 0.1 | −0.8 | −0.3 |
| ATP8B3 | −1.3 | −0.2 | 0.0 | −1.8 |
| ERCC6L | −1.3 | 0.8 | −0.1 | −0.3 |
| FAM117A | −1.3 | 0.1 | −0.9 | 0.2 |
| DDX39B | −1.3 | −0.3 | 1.4 | 1.4 |
| LOC100128071 | −1.3 | −0.5 | 0.5 | 1.3 |
| PLCE1 | −1.3 | 0.9 | 0.4 | −0.2 |
| ID4 | −1.3 | 0.2 | 0.0 | −0.3 |
| KIAA1377 | −1.3 | −0.5 | −0.5 | −0.4 |
| ZNF367 | −1.3 | −0.1 | 0.5 | 0.6 |
| SHISA3 | −1.3 | 0.3 | 1.2 | −1.2 |
| GUCY1B3 | −1.3 | 1.5 | −1.7 | −0.7 |
| SOCS1 | −1.3 | −1.3 | −1.2 | 0.0 |
| PBX1 | −1.3 | −0.2 | −0.5 | −0.5 |
| SLC7A14 | −1.3 | 0.2 | 3.2 | 3.3 |
| SNORD67 | −1.3 | −1.1 | −1.7 | −0.4 |
| LPPR4 | −1.3 | 0.5 | −0.7 | −0.5 |
| HES1 | −1.3 | −0.3 | −0.5 | 0.3 |
| HIST2H2BA | −1.3 | 1.4 | 0.3 | −0.9 |
| LNP1 | −1.3 | −0.1 | −0.7 | −0.5 |
| PCDH18 | −1.3 | −0.7 | −0.4 | −0.7 |
| KIF2C | −1.4 | 0.5 | −0.4 | −0.4 |
| GALNTL2 | −1.4 | −2.7 | −2.9 | 2.3 |
| KIF20B | −1.4 | 0.9 | −0.1 | −0.5 |
| GLIS1 | −1.4 | −7.0 | 2.2 | 0.5 |
| GLI3 | −1.4 | 0.4 | 0.1 | 0.0 |
| EYA4 | −1.4 | −0.1 | −2.0 | −1.2 |
| ROR2 | −1.4 | −5.7 | −1.5 | 0.7 |
| GPSM2 | −1.4 | 0.3 | −1.1 | −0.7 |
| BUB1 | −1.4 | 0.6 | −0.5 | −0.6 |
| GABRB1 | −1.4 | −1.8 | −2.6 | 1.2 |
| KIF18B | −1.4 | 0.8 | 0.1 | 0.2 |
| CDCA7 | −1.4 | −0.2 | 0.0 | −0.4 |
| SNORA14B | −1.4 | −1.3 | 0.2 | −3.4 |
| CCNF | −1.4 | 0.5 | −0.6 | −0.2 |
| MYBL1 | −1.4 | 1.1 | 1.0 | 0.0 |
| HSD17B2 | −1.4 | −0.1 | 0.2 | −0.6 |
| TSHZ1 | −1.4 | −2.3 | −0.2 | −0.1 |
| RAB27B | −1.4 | 0.2 | −1.1 | 0.5 |
| CDC25C | −1.4 | 0.5 | −0.7 | −0.6 |
| OIP5 | −1.4 | 0.4 | 0.0 | −0.3 |
| CDCA2 | −1.4 | 0.7 | −0.3 | −0.8 |
| CCDC89 | −1.4 | 0.7 | −1.5 | −2.1 |
| SNORD119 | −1.4 | 4.0 | −0.6 | 0.2 |
| AURKA | −1.4 | 0.6 | −0.7 | −0.7 |
| BMF | −1.4 | −1.2 | −0.6 | −0.7 |
| CENPF | −1.4 | 0.4 | −0.6 | −1.1 |
| CEBPD | −1.4 | 0.1 | 0.4 | 2.1 |
| PDE3B | −1.5 | −0.7 | 0.0 | −0.1 |
| ID2B | −1.5 | −0.3 | −0.3 | 1.3 |
| SMAD3 | −1.5 | −0.5 | −0.2 | −0.3 |
| HMMR | −1.5 | 0.4 | −0.8 | −1.0 |
| TOX | −1.5 | −3.7 | 0.6 | −0.4 |
| CCNB1 | −1.5 | 0.3 | −0.5 | −0.9 |
| KCNN2 | −1.5 | −2.5 | 0.1 | 1.4 |
| HJURP | −1.5 | 1.0 | −0.4 | −0.3 |
| PSRC1 | −1.5 | 0.8 | −1.0 | −0.8 |
| TOP2A | −1.5 | 0.5 | −0.6 | −1.0 |
| BTN3A3 | −1.5 | 0.0 | −0.5 | −0.4 |
| KIF11 | −1.5 | 0.5 | −0.2 | −0.2 |
| C3orf47 | −1.5 | −0.6 | 0.5 | −0.8 |
| C9orf140 | −1.5 | −0.1 | −0.7 | −0.5 |
| MYLIP | −1.5 | −0.6 | −0.3 | −1.3 |
| LOC400550 | −1.5 | −4.8 | 0.0 | −2.9 |
| LRIG3 | −1.5 | −0.4 | 0.1 | −0.5 |
| PIF1 | −1.5 | 0.4 | −1.3 | −1.8 |
| CDC20 | −1.5 | 0.3 | −0.7 | −0.2 |
| CDCA8 | −1.6 | 0.6 | −0.2 | −0.4 |
| FAM83D | −1.6 | 0.3 | −0.4 | −0.5 |
| MIR3685 | −1.6 | 1.6 | 0.5 | −0.4 |
| RGS2 | −1.6 | 0.3 | 0.3 | −0.6 |
| HLA-DMB | −1.6 | 0.9 | −1.4 | −1.9 |
| CA11 | −1.6 | −0.4 | −0.5 | −0.2 |
| DLL4 | −1.6 | 1.5 | 3.2 | 2.9 |
| EFCAB7 | −1.6 | −0.2 | 0.1 | −0.6 |
| SNORD98 | −1.6 | 0.1 | −1.4 | −5.0 |
| ID2 | −1.6 | −0.4 | −0.2 | 0.2 |
| TTK | −1.6 | 0.6 | −0.5 | −0.8 |
| GAS1 | −1.6 | −0.7 | −0.7 | −0.3 |
| MKI67 | −1.6 | 0.7 | −0.3 | 0.0 |
| SGOL2 | −1.6 | 0.8 | −0.5 | −0.6 |
| CCNA2 | −1.7 | 0.7 | −0.2 | −0.2 |
| CENPE | −1.7 | 0.6 | −0.7 | −1.2 |
| C10orf10 | −1.7 | −0.8 | −0.9 | 0.5 |
| LOC100128191 | −1.7 | 0.6 | 0.2 | 0.8 |
| HSD17B7 | −1.7 | 0.3 | −0.7 | −0.1 |
| TMEM119 | −1.7 | 0.7 | −2.6 | 0.5 |
| HOXA5 | −1.7 | −0.8 | 0.5 | 0.1 |
| SLC40A1 | −1.7 | −0.2 | 0.5 | −0.6 |
| FAM111B | −1.7 | 0.3 | 0.3 | 0.2 |
| CXCL1 | −1.7 | 0.5 | 2.6 | 0.1 |
| C8orf4 | −1.7 | 1.2 | −0.2 | −1.4 |
| LMNB1 | −1.7 | 0.2 | 0.2 | −0.4 |
| NEK2 | −1.7 | 0.3 | −0.6 | −0.9 |
| IFIT2 | −1.7 | 0.2 | −0.1 | 0.1 |
| SLC16A14 | −1.7 | 0.4 | −1.1 | −0.5 |
| HOXA3 | −1.7 | −0.3 | 3.2 | 1.6 |
| NDC80 | −1.8 | 0.5 | −0.2 | −0.9 |
| DEPDC1B | −1.8 | 0.6 | −0.3 | −0.4 |
| ZNF608 | −1.8 | 0.1 | −0.6 | −0.4 |
| PLK1 | −1.8 | 0.6 | −0.8 | 0.1 |
| CDKN2C | −1.8 | 0.2 | 0.2 | 0.1 |
| PPAP2B | −1.8 | −0.2 | 0.2 | −2.7 |
| ATOH8 | −1.8 | −1.1 | −0.1 | 0.3 |
| MIR26A2 | −1.8 | −2.5 | 4.7 | 0.5 |
| KIF18A | −1.78 | 0.38 | −0.37 | −0.51 |
| METTL7A | −1.78 | 1.21 | −1.61 | −1.04 |
| KIT | −1.79 | −0.43 | −0.36 | −0.05 |
| SLC12A8 | −1.8 | 0.41 | −0.2 | −0.12 |
| BUB1B | −1.82 | 0.48 | −0.42 | −0.62 |
| TBX5 | −1.82 | −0.33 | −0.07 | 1.94 |
| C5orf4 | −1.83 | −0.9 | 1.65 | 0.08 |
| ASPM | −1.83 | 0.36 | −0.79 | −0.97 |
| PDE5A | −1.83 | −0.72 | −0.01 | −1.14 |
| MXD3 | −1.84 | −0.14 | −0.84 | −0.55 |
| BHLHE41 | −1.84 | −3.23 | −0.15 | 0.05 |
| SOX5 | −1.85 | 0 | −0.05 | −0.37 |
| CENPA | −1.88 | 0.47 | −0.63 | −1.01 |
| RPS29 | −1.94 | −3.55 | −0.47 | −0.83 |
| BORA | −1.94 | 0.51 | −0.39 | −0.75 |
| DLGAP5 | −1.95 | 0.53 | −0.85 | −1.24 |
| SNORD116-8 | −1.97 | −3.55 | −2.86 | 1.67 |
| SNORD42B | −1.98 | 2.15 | 0.66 | 1.57 |
| KIF20A | −1.99 | 0.66 | −0.73 | −0.75 |
| SMAD6 | −1.99 | 0.01 | 0.66 | 1.91 |
| HCG25 | −2 | −0.18 | 0.25 | −0.28 |
| IFIT1 | −2 | 1 | −0.56 | −1.17 |
| LOC100507547 | −2.01 | −0.03 | −0.62 | −2.29 |
| RASL11A | −2.03 | −0.23 | 1.37 | 0.06 |
| SNORA60 | −2.04 | −2.71 | 0.2 | −1.73 |
| MIR3186 | −2.08 | −0.5 | −0.37 | −2.47 |
| HOXA2 | −2.12 | 0.51 | 0.08 | 1.08 |
| TMEM155 | −2.19 | 1.22 | 2.96 | 0.93 |
| MIR3911 | −2.25 | −3.52 | −0.37 | −0.74 |
| MIR1281 | −2.25 | −0.79 | 0.44 | −0.68 |
| PDE7B | −2.27 | 0.04 | −0.93 | −0.46 |
| HCG4B | −2.36 | −4.49 | −0.76 | −1.88 |
| MIR3153 | −2.37 | 1.61 | −0.39 | 0.2 |
| SNORD116-15 | −2.4 | −2.57 | 0.26 | 0.53 |
| SNORD116-27 | −2.4 | 1.02 | 0.26 | −3.84 |
| POU5F1 | −2.48 | 0.58 | 0.58 | 0.75 |
| NR4A2 | −2.65 | −0.18 | 0.41 | 0.38 |
| MAP2K6 | −2.69 | −0.54 | −0.82 | −0.77 |
| MIR4754 | −2.76 | −0.53 | 1.53 | 3.48 |
| NR4A3 | −2.82 | 0.28 | 0.22 | 0.2 |
| HSP90AA6P | −2.89 | 1.4 | 0.15 | 2.52 |
| HOXA6 | −2.96 | −1.11 | 0.17 | 1.44 |
| HTR2B | −3.1 | 4.67 | −0.57 | 0 |
| ADAMTS15 | −3.46 | 0.85 | 3.3 | 1.08 |
| MIR199A1 | −5.01 | 3.91 | 0.27 | 3.28 |
| MIRLET7I | −5.46 | −2.53 | −2.78 | 0.26 |
| SNORD58C | −5.47 | 2.05 | 2.88 | −0.67 |

Following functional annotation analysis, there was a significant enrichment of genes involved in transcription regulation and RNA metabolism, implying that these pathways were dysregulated upon damage in EwS. This is because EWS-FLI1 drives an aberrant transcriptional program, and loss of EWSR1 function leads to RNA splicing defects. Cross-examination of a genome-wide RNAi survival screen was done in Drosophila Kc167 cells exposed to various damaging agents (etoposide, methyl methane sulfonate (alkylating damage) and bleomycin (radiomimetic)), highlighted RNA metabolism as a consistent top hit in damage survival pathways (FIG. 8, Table 6). FIG. 8 is a graphical representation depicting the degree of inhibition of phospho-Ser2 deposition by CDK9 kinase activity by either buffer (vehicle) or recombinant EWSR1/FUS proteins on two independent substrates (CDKtide-shorter triheptad repeat peptide, or CTD—the C terminal domain of RNAPII). This implies that damage-induced changes in transcriptional regulation and RNA processing are critical and conserved damage survival pathways.

Table 6 The top 5% hits in the Drosophila kc167 RNAi screens. Data was collected as percent survival upon damage induction (3 days) for each RNAi. The loci that mapped to NCBI gene IDs are listed.

TABLE 6

| MMS | Bleomycin | Etoposide |
|---|---|---|
| Survival Hits | Survival Hits | Survival Hits |
| Aats-his | 5-HT1B | Aats-ala |
| Aats-val | Aats-ala | Aats-his |
| ac | Aats-trp | Aats-val |
| AdoR | Aats-val | Ac13E |
| Aly | ac | Ada |
| aop | Act5C | Adk2 |
| Aos1 | Adk2 | alpha-Est7 |
| aret | Aldh-III | alphaPS4 |
| aur | alpha-Est6 | alphaTub85E |
| betaTry | alphaTub84B | AlstR |
| bnl | alphaTub85E | aop |
| br | AlstR | aret |
| brn | Aly | aur |
| btz | aop | Bub1 |
| Cdc27 | aret | BubR1 |
| CDC45L | Argk | Bx42 |
| Cf2 | betaTub56D | caz |
| CG10178 | Brf | Cdc27 |
| CG10254 | Bub3 | Cdep |
| CG10754 | BubR1 | Cdk7 |
| CG11913 | Bx42 | Cdk9 |
| CG12279 | cdc16 | Cf2 |
| CG13298 | cdc2 | CG10133 |
| CG14641 | Cdc27 | CG10425 |
| CG14899 | CDC45L | CG10754 |
| CG17266 | Cdk7 | CG10996 |
| CG18672 | CG10178 | CG11070 |
| CG18673 | CG10581 | CG11134 |
| CG2685 | CG10754 | CG12267 |
| CG31002 | CG10909 | CG12279 |
| CG31158 | CG10960 | CG12398 |
| CG31687 | CG11070 | CG12602 |
| CG31688 | CG11255 | CG13384 |
| CG32251 | CG1129 | CG13397 |
| CG3290 | CG11425 | CG13890 |
| CG3292 | CG11438 | CG13900 |
| CG33138 | CG11874 | CG14023 |
| CG33671 | CG11913 | CG14516 |
| CG34139 | CG11943 | CG14899 |
| CG40351 | CG11985 | CG14934 |
| CG42304 | CG12237 | CG17119 |
| CG42400 | CG12279 | CG17266 |
| CG42458 | CG13298 | CG18789 |
| CG42575 | CG13384 | CG2964 |
| CG5380 | CG13397 | CG30105 |
| CG5432 | CG14023 | CG30463 |
| CG5857 | CG14463 | CG3061 |
| CG6154 | CG14542 | CG31547 |
| CG6262 | CG14550 | CG32251 |
| CG6418 | CG16733 | CG32626 |
| CG6453 | CG17119 | CG33138 |
| CG7375 | CG17266 | CG33158 |
| CG8771 | CG18003 | CG34139 |
| CG9350 | CG18193 | CG3605 |
| CG9769 | CG18789 | CG42304 |
| CG9799 | CG2972 | CG42400 |
| Chrac-14 | CG31002 | CG42575 |
| Cht8 | CG31158 | CG4988 |
| Cip4 | CG3194 | CG5044 |
| Cp1 | CG32251 | CG5613 |
| CycE | CG32812 | CG6194 |
| CycT | CG33138 | CG6227 |
| Cyp6a2 | CG34139 | CG6262 |
| dco | CG3446 | CG6847 |
| decay | CG4119 | CG7026 |
| Dgk | CG42304 | CG7185 |
| dm | CG42400 | CG7329 |
| DNApol-delta | CG42458 | CG7757 |
| dnc | CG42575 | CG8029 |
| Droj2 | CG4752 | CG8117 |
| Dscam | CG5018 | CG9065 |
| Ef1alpha100E | CG5432 | CG9107 |
| eIF-3p66 | CG5550 | CG9232 |
| eIF-4E | CG6262 | CG9452 |
| eIF3-S8 | CG6418 | CG9471 |
| eIF4G | CG6737 | CG9547 |
| Eip75B | CG6914 | CG9799 |
| emc | CG7185 | CG9906 |
| Fip1 | CG8029 | CG9914 |
| foxo | CG8177 | CG9940 |
| fu | CG9107 | Cha |
| fzy | CG9232 | Cyp1 |
| Gclc | CG9547 | Cyp6a19 |
| Gclm | Cha | Cyp6a22 |
| gfzf | Cht7 | Cyp6a8 |
| Glycogenin | Csas | DAAM |
| Gnf1 | CstF-64 | decay |
| Gyc-89Da | CtBP | dm |
| hay | CycB | dnc |
| Hex-t1 | cyp33 | Dsp1 |
| Hr38 | Cyp6a17 | E2f |
| hrg | Cyp6a19 | eas |
| Hs3st-A | Cyp6a2 | Ef1alpha48D |
| Hsc70Cb | Cyp6a22 | eIF-4E |
| Hsp70Aa | Cyp6a8 | Elf |
| Hsp70Ab | decay | emc |
| Hsp70Ba | Dgk | epsilonCOP |
| Hsp70Bb | dm | epsilonTry |
| Hsp70Bbb | DNApol-epsilon | Fen1 |
| Hsp70Bc | dnc | Fib |
| Hsp83 | Droj2 | foxo |
| hyd | Ef1alpha48D | fu12 |
| kay | egg | Fur1 |
| l(1)10Bb | eIF-4E | fzy |
| Lgr3 | eIF-5A | garz |
| mago | epsilonCOP | GckIII |
| mam | Fas2 | Gip |
| Mat1 | Fib | GlcAT-P |
| Mdr65 | foxo | GlyP |
| Med | Fs(2)Ket | Gyc-89Da |
| MED1 | fu | hay |
| Mov34 | fu12 | HDAC6 |
| mRNA-capping- | fzy | HmgZ |
| enzyme | | |
| msi | Gef26 | Hnf4 |
| ND5 | Glu-RIB | Hs3st-A |
| Nf-YA | GlyP | Hsc70-4 |
| Nf-YC | grk | Hsp70Aa |
| Nop56 | Hex-t1 | Hsp70Ab |
| Nup107 | Hml | Hsp70Ba |
| Nup133 | hrg | Hsp70Bb |
| Nup58 | Hsc70-1 | Hsp70Bbb |
| Nup62 | Hsp70Aa | Hsp70Bc |
| Nup75 | Hsp70Ab | IP3K1 |
| Octbeta3R | Hsp70Ba | Ipp |
| Pabp2 | Hsp70Bb | Irbp |
| Patr-1 | Hsp70Bbb | l(1)G0007 |
| Pcaf | Hsp70Bc | lat |
| Pcf11 | Hsp83 | laza |
| Pdp1 | Ilp2 | M(2)21AB |
| Pect | jhamt | mAcR-60C |
| PHGPx | l(1)G0020 | mam |
| phl | l(1)G0148 | Mcm2 |
| pont | l(2)k09022 | Mdr49 |
| Pros26.4 | lat | Med |
| Pros54 | Egr3 | Mlc-c |
| PyK | M(2)21AB | Mocs2 |
| Rbf | mam | Mov34 |
| rdx | Mcm7 | mRNA-capping- |
| | | enzyme |
| Rho1 | Mdr49 | Ncc69 |
| rl | Mdr50 | NELF-B |
| RnpS1 | mGluRA | Nf-YA |
| RnrS | Mo25 | Nf-YC |
| Roc1a | Mocs2 | nrv3 |
| Rpb10 | Mov34 | Nup107 |
| Rpb4 | mRNA-capping- | Nup153 |
| | enzyme | |
| Rpb5 | msi | Nup98 |
| Rpb7 | NAT1 | oho23B |
| Rpb8 | ND5 | orb |
| RpII140 | Nf-YC | Orc1 |
| RpII15 | Not1 | Pak |
| RpII18 | Nup107 | Pcaf |
| RpL12 | Nup133 | Pcf11 |
| RpL18 | Nup153 | Pde11 |
| RpL19 | Nup98 | Pdp1 |
| RpL22 | oa2 | Pect |
| RpL31 | Orc1 | Pka-C1 |
| RpL7 | Orc4 | pont |
| Rpn1 | Past1 | PP2A-B′ |
| Rpn5 | pav | Pp2B-14D |
| Rpn6 | Pcaf | Pp2C1 |
| Rpn7 | Pcf11 | ppan |
| RpS12 | Pde1c | Pros28.1 |
| RpS15Aa | Pdp1 | Psf1 |
| RpS15Ab | Pect | Rab11 |
| RpS18 | Pgant35A | Rbp2 |
| RpS19b | Pgk | rdx |
| RpS25 | phl | rin |
| RpS3 | Pomp | RnrS |
| RpS30 | pont | Roc1a |
| RpS3A | PP2A-B′ | Rpb5 |
| RpS9 | pr | RpL10Aa |
| Rpt1 | Pros26 | RpL12 |
| Rpt3 | Pros28.1 | RpL13 |
| Rpt3R | Prosbeta5R | RpL14 |
| Rpt4 | Pvr | RpL26 |
| rut | r-1 | RpL41 |
| sdt | Rab11 | RpL7A |
| sec63 | raptor | Rpn11 |
| SF2 | Rh2 | Rpn6 |
| sfl | Rho1 | Rpn9 |
| sima | RhoGAP93B | RpS14a |
| Sin | Rm62 | RpS14b |
| Sin3A | RnrL | Rpt1 |
| sinah | RnrS | Rrp6 |
| siz | Roc1a | Rtc1 |
| Sk1 | RpA-70 | RunxB |
| skpA | RpII18 | rut |
| slmb | RpL12 | sec15 |
| Slu7 | RpL13 | Sec61alpha |
| Smr | RpL18 | SF2 |
| SNF1A | RpL19 | shrb |
| sns | RpL23 | Sin |
| Sos | RpL39 | Slu7 |
| spt4 | RpL7A | Smr |
| Spt5 | RpLP1 | snf |
| Ssl1 | RpLP2 | Socs16D |
| stg | Rpn1 | sog |
| T-cp1 | Rpn11 | spel1 |
| tacc | Rpn12 | Sply |
| Taf1 | Rpn2 | Spt5 |
| Taf10 | Rpn3 | Spx |
| Taf10b | Rpn5 | sqd |
| Taf4 | Rpn6 | sws |
| Taf5 | Rpn7 | T-cp1 |
| Taf7 | Rpn9 | Taf8 |
| Tbp-1 | RpS12 | Tbp |
| Tcp-1eta | RpS13 | tex |
| tex | RpS14a | TfIIFalpha |
| Tfb1 | RpS14b | Tm2 |
| TfIIB | RpS3 | trr |
| TfIIEalpha | RpS9 | Tsp29Fa |
| TfIIEbeta | Rpt1 | tsr |
| TfIIFalpha | Rpt4 | tw |
| TfIIFbeta | rut | U2af50 |
| tws | sc | vih |
| U2A | sdt | vri |
| Uba2 | sec13 | wbl |
| Ugt86Dd | sec15 | wee |
| usp | Sec61alpha | Wnt5 |
| Vha44 | sec63 | |
| vri | shrb | |
| wda | sima | |
| wdb | Sin | |
| Wnt5 | siz | |
| yin | skpA | |
| | Smr | |
| | smt3 | |
| | snf | |
| | SNF1A | |
| | Spt5 | |
| | Spx | |
| | stg | |
| | Su(var)2-10 | |
| | Syx8 | |
| | tacc | |
| | Taf1 | |
| | Taf10 | |
| | Taf10b | |
| | Taf2 | |
| | Taf4 | |
| | Taf7 | |
| | TER94 | |
| | TfIIEbeta | |
| | TfIIFalpha | |
| | trpgamma | |
| | trr | |
| | tsr | |
| | U2A | |
| | Uba2 | |
| | Vha14 | |
| | vih | |
| | Vps25 | |

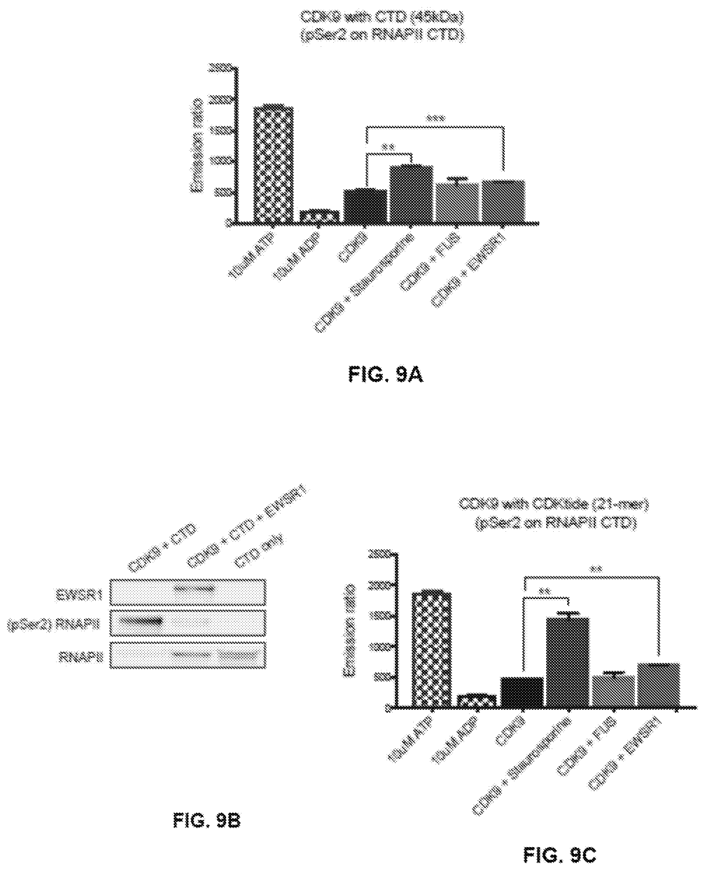

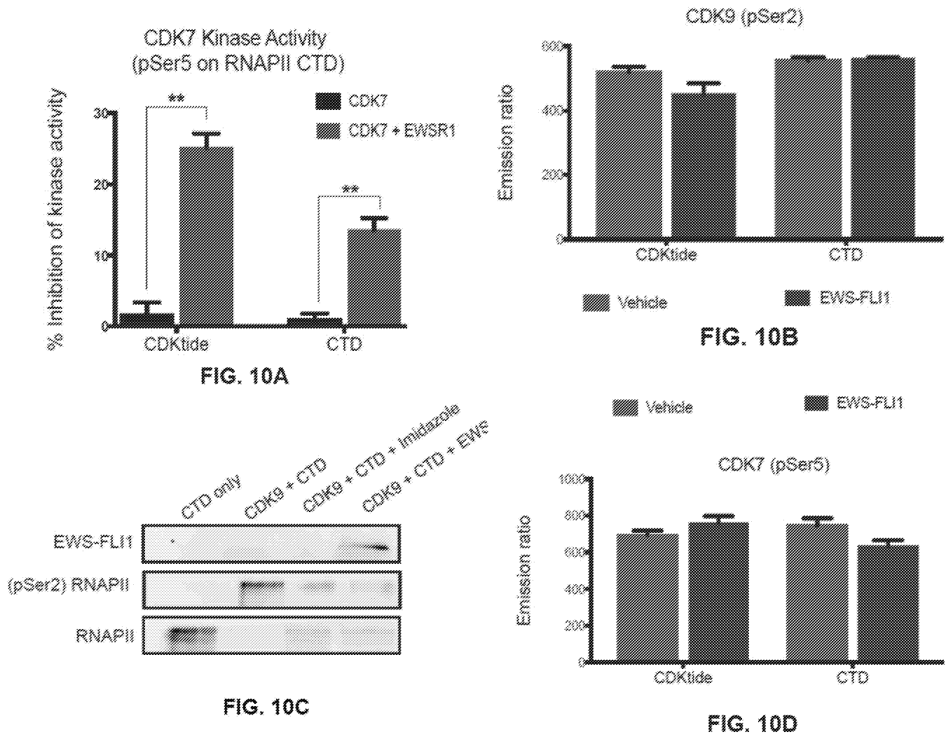

EWS-FLI1 and EWSR1 are known to interact with each other as well as with various subcomponents of the transcription machinery. While EWSR1 is largely believed to act as an adaptor molecule coupling the transcription and splicing machinery, it has been suggested that EWS-FLI1 acts in a dominant-negative manner to interfere with splicing. However, the role of these two proteins in directly controlling RNAPII activity has not been actively studied. The largest subunit of RNAPII is hyperphosphorylated at Ser2 and Ser5 of the heptapeptide repeats in the C-terminal domain (CTD) during active transcription, Ser5 phosphorylation (by CDK7/cyclin H in the TFIIH machinery) occurring early during initiation and Ser2 phosphorylation (by CDK9/cyclin T1) triggering elongation. FUS, another TET family protein with high homology to and interacting with EWSR1, regulates RNAPII phosphorylation to control transcription rates and splicing. Therefore, the altered transcription regulation observed in EwS could be due to EWS-FLI1 interfering with wild type EWSR1 in regulating transcription. In an in vitro kinase assay using purified recombinant proteins, similar to FUS, EWSR1 inhibited the phosphorylation of RNAPII CTD by CDK9 (FIGS. 9A-9C and FIGS. 10A-10C) and to a lesser extent by CDK7 (FIG. 10D). FIGS. 9A-9C is a set of graphical representations of the degree of inhibition of phospho-Ser2 deposition by CDK9 kinase activity by either buffer (vehicle) or recombinant EWSR1/FUS proteins on two independent substrates (CDKtide-shorter triheptad repeat peptide, or CTD—the C terminal domain of RNAPII). FIG. 9A is a graphical representation of the effects of inhibition of CDK9 kinase activity on RNAPII CTD fragment under various treatments—vehicle treated, staurosporine (a chemical pan-kinase inhibitor), recombinant FUS protein or EWSR1 protein. The height of the bars is inversely proportional to the amount of ADP produced by the kinase reaction thus indicating level of inhibition. 10 μM ATP and 10 μM ADP were used as internal negative and positive controls for the assay. FIG. 9B is a photograph of an immunoblot depicting the phosphorylation of RNAPII CTD fragment used as the substrate in FIG. 9A. Recombinant EWSR1 and hypophosphorylated RNAPII are also displayed. FIG. 9C is a graphical representation of the effects of the inhibition of CDK9 kinase activity on a smaller tri-heptad repeat of RNAPII CTD (CDKtide, 21-mer) under the same conditions as in FIG. 9A. FIG. 10A is a graphical representation of the percentage of inhibition of CDK7 kinase activity (phosphorylation on Ser5 of RNAPII CTD repeats) with or without EWSR1 on the two substrates. FIG. 10B is a graphical representation of the effects of inhibition of CDK9 kinase activity by buffer (vehicle) or recombinant EWS-FLI1 protein on the two RNAPII CTD substrates. FIG. 10C is a photograph of an immunoblot confirming the kinase activity measured by the assay. The blots show amount of recombinant EWS-FLI1 protein and its effect on the phosphorylation of the bigger CTD domain fragment (45 kDa). Hypophosphorylated RNAPII is also indicated. Statistical significance is evaluated relative to vehicle treated (CDK9 only) conditions. **P<0.005, bar graph depicts mean with SE, n=3 or 4. FIG. 10D is a graphical representation of the effects of inhibition of CDK7 kinase activity by buffer (vehicle) or recombinant EWS-FLI1 protein on the two RNAPII CTD substrates.

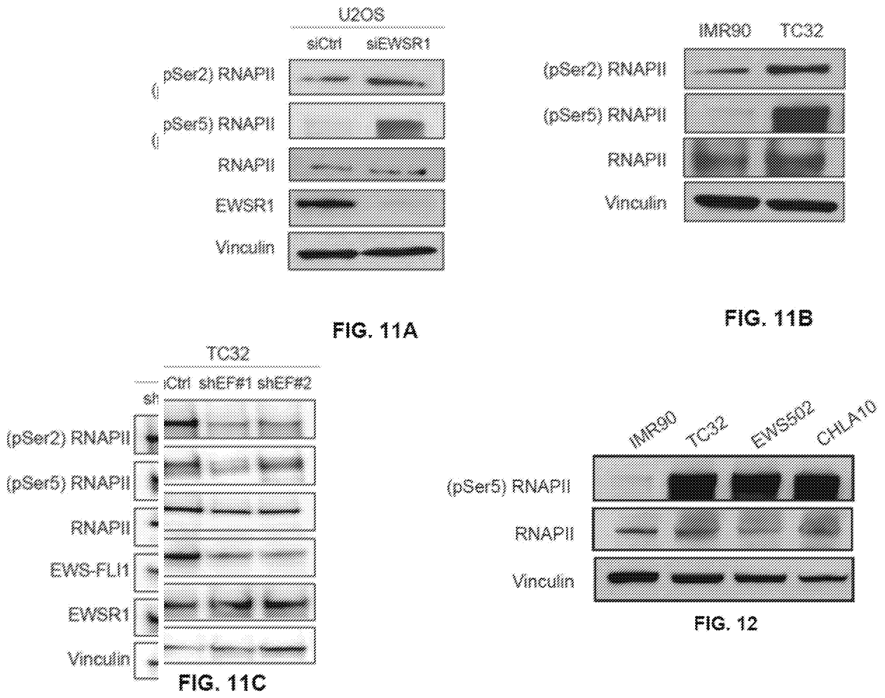

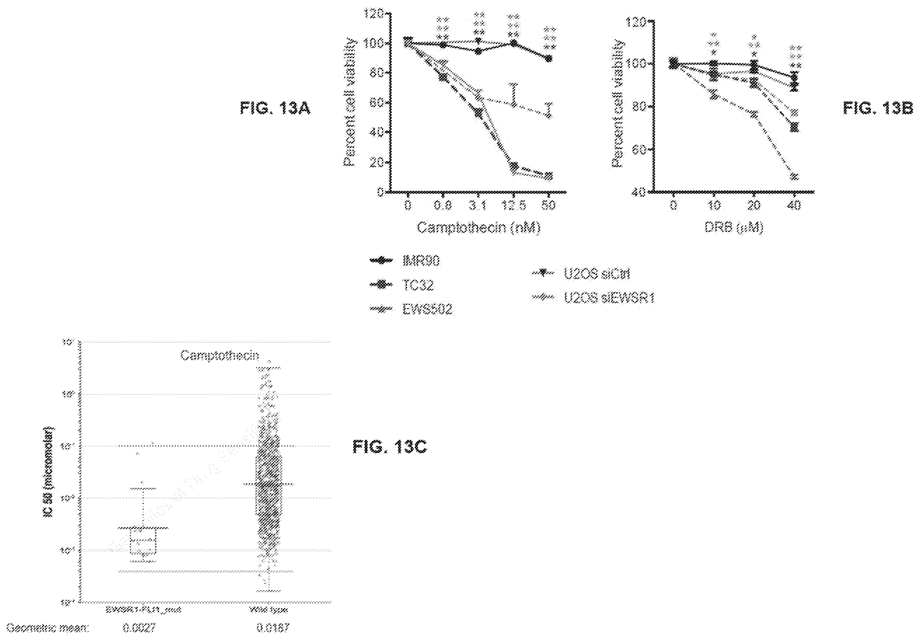

EWS-FLI1, on the other hand, caused no significant change in kinase activity (FIGS. 10C-10E) suggesting that it might not have the same inhibitory potential on RNAPII CTD phosphorylation. Phospho-RNAPII was evaluated in cell lysates. Loss of EWSR1 in U2OS cells caused a significant increase in active RNAPII confirming the results from the kinase assay (FIG. 11A). FIG. 11A is a photograph of an immunoblot depicting levels of phosphorylated Ser2/Ser5 at CTD of RNAPII in U2OS cells with and without EWSR1 knockdown. FIG. 11B is a photograph of an immunoblot depicting levels of phosphorylated Ser2/Ser5 at CTD of RNAPII in control IMR90 cells versus TC32 cells. Immunoblotting of EwS cell lysates indicated even higher levels of both phospho-Ser2 and phospho-Ser5 RNAPII compared to IMR90 (FIG. 11B and FIG. 12). FIG. 11C is a photograph of an immunoblot depicting levels of phosphorylated Ser2/Ser5 at CTD of RNAPII in TC32 cells with and without EWS-FLI1 knockdown. Depletion of EWS-FLI1 in TC32 cells also resulted in a significant decrease in RNAPII phosphorylation (FIG. 11C). The levels of wild-type EWSR1 were not affected by EWS-FLI1 knockdown. EWS-FLI1 increased basal levels of transcription, either directly or indirectly by interfering with EWSR1 activity. FIG. 12 is a photograph of an immunoblot depicting phosphorylated RNAPII in a panel of EwS cell lines. Accordingly, EwS and EWSR1-depleted cell lines were also much more sensitive to camptothecin (topoisomerase I inhibitor) and 5,6-Dichlorobenzimidazole 1- [symbol] -D ribofuranoside (DRB) (FIGS. 13A-13B), which block transcription. EWS-FLI1 dependency on camptothecin sensitivity was validated in an independent pan-cancer dataset (FIG. 13C). FIG. 13A is a graphical representation of a cytotoxicity profile of control, EwS and EWSR1-depleted cells exposed to increasing doses of camptothecin, measured 72 hours after treatment. FIG. 13B is a graphical representation of a cytotoxicity profile of control, EwS and EWSR1-depleted cells exposed to increasing doses of DRB, measured 72 hours after treatment. FIG. 13C is a box-and-whisker plots depicting the IC50 levels of etoposide or mitomycin in EWS-FLI1 mutant (15 cell lines) versus pan-cancer (132 cell lines). The brown lines depict the range of screening concentrations of the drug. The red lines represent the geometric mean of the drug concentration. *P<0.05, **P<0.005, the colors of the * match that used for the cell line in the multi-cell line cytotoxicity plots and were tested against control. Significance for U2OS siEWSR1 cytotoxicity plots was evaluated relative to U2OS siCtrl. Line graphs display means with SE, n=4.

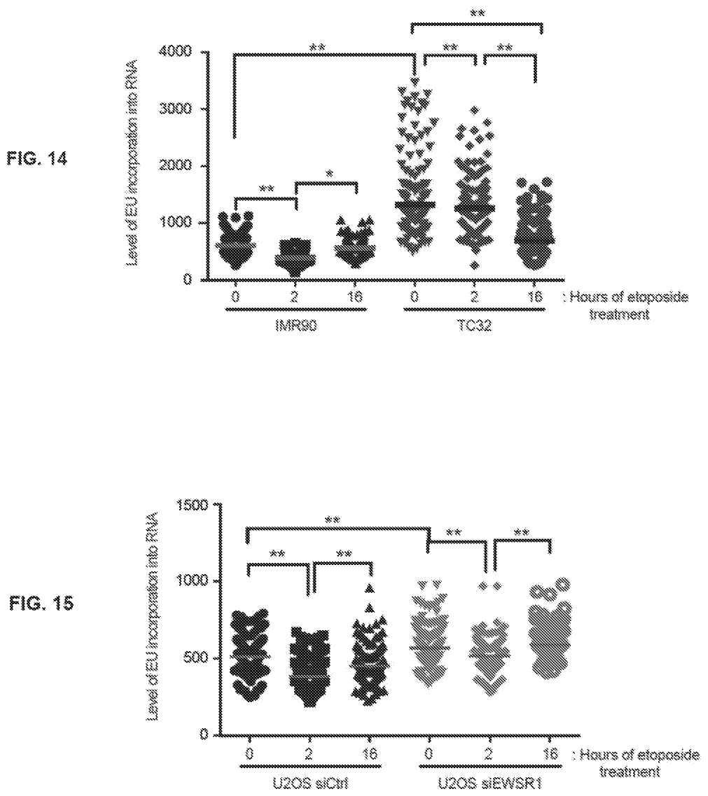

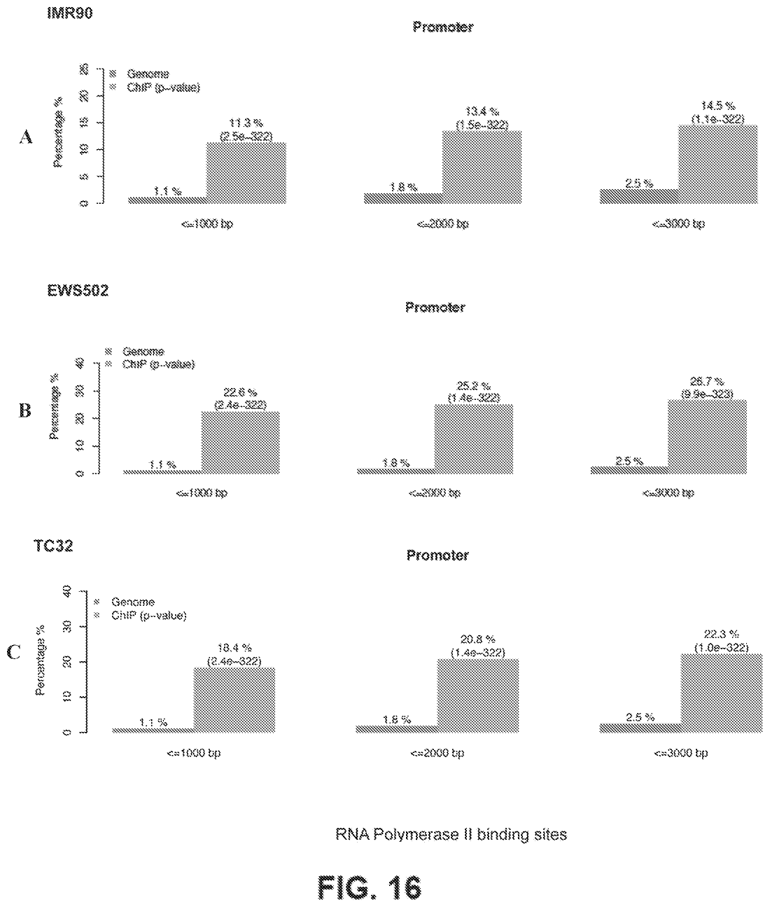

DNA damage induces a global suppression of transcription (involving BRCA1) followed by gradual recovery. As the damage-dependent transcriptional program appeared to be absent in EwS, transcription response and recovery following etoposide exposure were evaluated by measuring incorporation of a uridine analog, ethynyl uridine (EU) into RNA. Transcriptional activity was evaluated in cells exposed to etoposide for 2 or 16 hours. Transcription restart was measured based on level of EU uptake over a 30 min labeling period. The Y-axis represents relative fluorescence units from the EU-Click-iT assay. FIG. 14 is a graphical representation of the results from the transcription restart assay measured in in control IMR90 cells versus TC32 cells. FIG. 15 is a graphical representation of the results from the transcription restart assay measured in U2OS cells transfected with either scrambled siRNA or EWSR1 siRNA. *P<0.05, **P<0.005, the colors of the * match that used for graphs display means with SE, n=4. IMR90 cells demonstrated the characteristic decrease in EU incorporation two hours post-damage followed by recovery at 16 hours. In contrast, TC32 had a significantly higher basal level transcription and a much delayed loss of transcription. EWSR1 knockdown in U2OS also resulted in increased basal transcription and a muted response to damage at two hours (FIG. 15). Finally, as a direct measure of transcriptional activity, RNAPII binding was measured across the genome by chromatin immunoprecipitation sequencing (ChIP-seq). Striking differences were noted in the binding profiles of RNAPII between IMR90 and EwS cells (FIGS. 16A-16C). FIG. 16A is a histogram of the percentage occupancy of RNAPII in the promoter regions (<=1000, <=2000 and <=3000 bp upstream of the transcription start site) in RNAPII ChIP sample of IMR90 cells. FIG. 16B is a histogram of the percentage occupancy of RNAPII in the promoter regions (<=1000, <=2000 and <=3000 bp upstream of the transcription start site) in RNAPII ChIP sample of EWS502 cells. FIG. 16C is a histogram of the percentage occupancy of RNAPII in the promoter regions (<=1000, <=2000 and <=3000 bp upstream of the transcription start site) in RNAPII ChIP sample of TC32 cells. Most importantly, there was a nearly two-fold increase in the level of promoter-bound RNAPII in EwS. Taken together, the above results suggest that either loss of EWSR1 or expression of EWS-FLI1 results in aberrant transcription regulation, particularly in response to DNA damage.

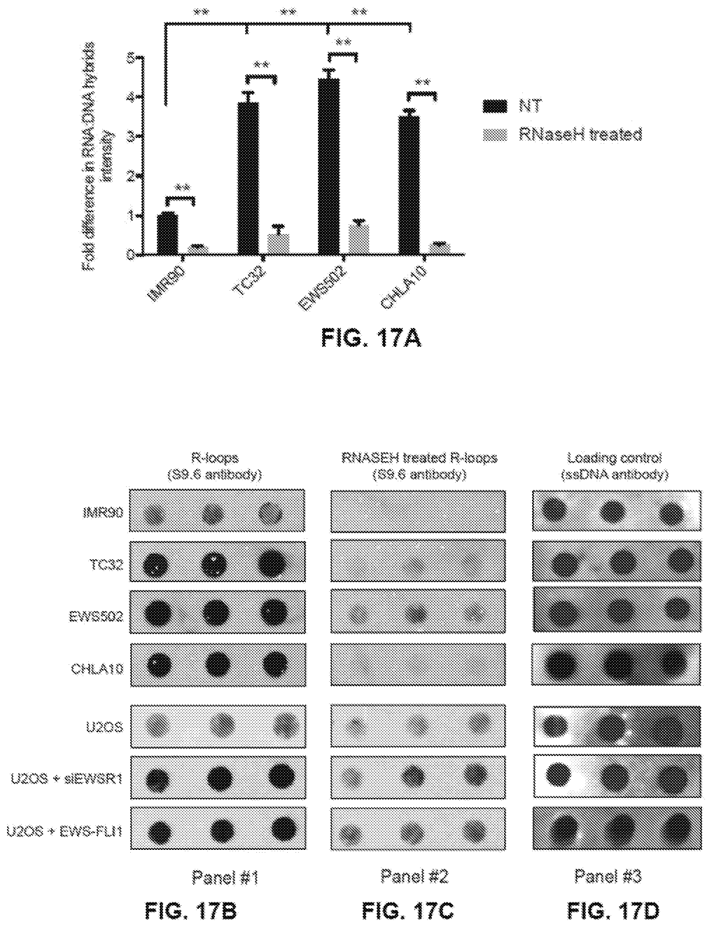

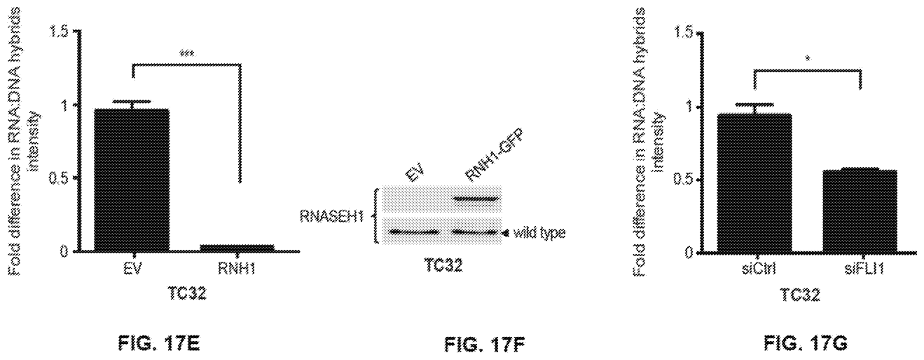

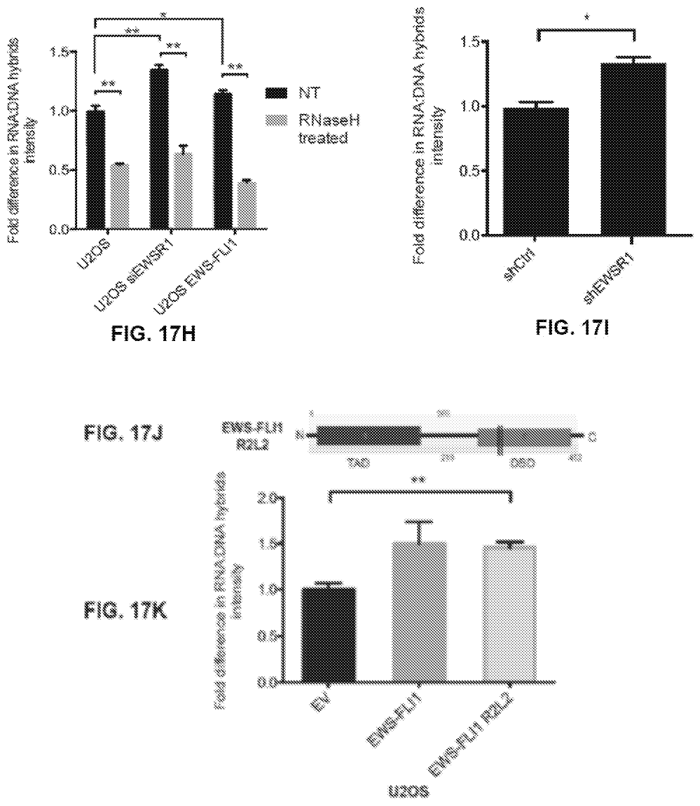

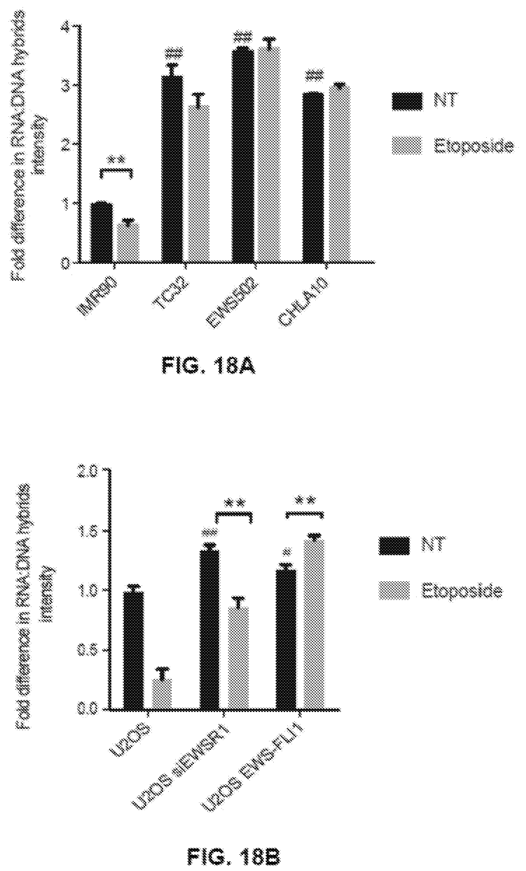

EWSR1 is necessary to prevent accumulation of R-loops Alterations in transcription regulation could result in the accumulation of R-loops. EWSR1 and EWS-FLI1 appear to have a role in regulating R-loop accumulation: both proteins 1) regulate RNAPII elongation; 2) bind G-rich single stranded DNA sequences, and 3) interact with the splicing machinery to control alternative splice events, each phenomenon conducive to R-loop formation. When a RNA:DNA hybrid specific S9.6 antibody was used to probe genomic DNA, EwS cell lines displayed nearly four-fold higher levels of R-loops (FIGS. 17A-17D).

FIG. 17A is a graphical representation of the quantification of R-loops detected using S9.6 antibody on dot blot of genomic DNA from IMR 90, TC32, EWS502, and CHLA10 cells. Fold-difference was measured relative to untreated IMR90. Grey bars measure signal obtained after RNaseH treatment and indicate specificity of signal. NT: No Treatment. FIGS. 17B-17D are representative dot blot indicating levels of RNA:DNA hybrids or single-stranded DNA (ssDNA). The dot blots of FIGS. 17B and 17C were probed with S9.6 antibody. In FIG. 17B, Panel 1 contains genomic DNA and in FIG. 17C, Panel 2 is genomic DNA that had been further treated with RNaseH enzyme. In FIG. 17D, Panel 3 has denatured genomic DNA that was probed with ssDNA antibody and serves as a loading control. Each dot serves as a technical replicate.

R-loop signal was abrogated upon pre-treatment of the DNA with RNaseH indicating specificity of the antibody. As an additional rescue experiment, cells were transfected with an RNASEH1 expression vector, which resulted in a substantial decrease in R-loop signal (FIGS. 17E-17F). FIG. 17E is a graphical representation of quantification from a dot blot measuring levels of RNA:DNA hybrids in DNA harvested from TC32 that have been transfected either with an empty vector (EV) or RNASEH1 (RNH1). R-loop signal was normalized to its loading control (ssDNA). FIG. 17F is a photograph of an immunoblot that indicates the RNASEH1 transfection efficiency. A decrease in R-loop signal was also observed in TC32 cells after EWS-Fill knockdown (FIG. 17G). FIG. 17G is a graphical representation of quantification of RNA:DNA hybrids in DNA harvested from TC32 cells that have been depleted of EWS-LI1 (siFLI1). For direct comparison, a syngeneic model was used, which consisted of U2OS cells with either 2 independent knockdown methods of EWSR1 or expression of EWS-FLI and respective controls (FIGS. 17H and 17I). FIG. 17H is a graphical representation of the quantification of R-loops detected using S9.6 antibody on dot blot of genomic DNA from U2OS cells transfected with either siRNA against EWSR1 or EWS-FLI1 expression vector. Fold-difference was measured relative to untreated IMR90. Grey bars measure signal obtained after RNaseH treatment and indicate specificity of signal. NT: No Treatment. FIG. 17I is a graphical representation of quantification of RNA:DNA hybrids in U2OS cells with shRNA mediated depletion of EWSR1. FIG. 17J is a schematic of the EWS-FLI1 R2L2 construct. Arginine residues 383 and 386 (indicated by black vertical bars) in EWS-FLI1 are converted to Leucine to render the fusion oncogene deficient in DNA binding. FIG. 17K is a graphical representation of quantification of RNA:DNA hybrids in U2OS cells expressing either empty vector (EV), EWS-FLI1 or EWS-FLI1 R2L2. *P<0.05, **P<0.005, bar graphs depict means with SE, n=4. Importantly, wild type EWSR1 was not simultaneously silenced when EWS-FLI1 was expressed. Both genetic manipulations caused accrual of R-loops in U2OS cells. A DNA-binding mutant form of EWS-FLI1 also resulted in similar accrual of R-loops (FIG. 17K), implicating the importance of the N-terminal protein interaction domain in EWSR1 and EWS-FLI1 in promoting R-loop accumulation.

With global decline in transcription following damage, a concomitant decrease in R-loop signal with etoposide (FIGS. 18A and 18B) was observed in IMR90. FIG. 18A is a graphical representation of the fold change in R-loop signal after damage (etoposide, 6 hours) in control vs. EwS cells. FIG. 18B is a graphical representation of the fold change in R-loop signal after damage (etoposide, 6 hours) in U2OS cells with either EWSR1 depletion or EWS-FLI1 expression. The # symbol indicates significance of EwS relative to untreated IMR90 or transfections relative to U20S. NT: No Treatment.

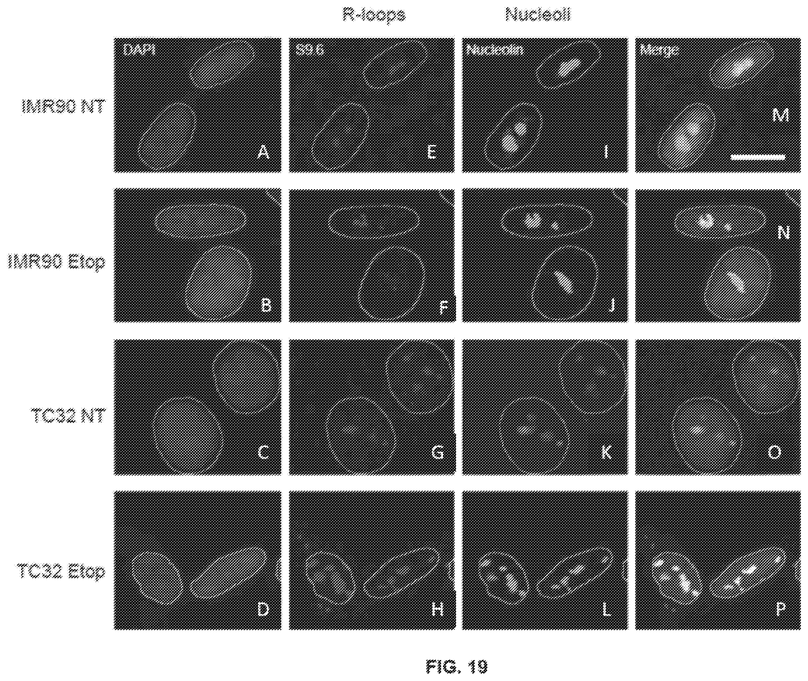

Remarkably, EwS cell lines and U2OS with EWS-FLI1 expression did not display similar damage-induced reduction in R-loops, substantiating the EU-incorporation data (FIGS. 14 and 15). These results were corroborated by immunofluorescence analysis (FIGS. 19A-19P).

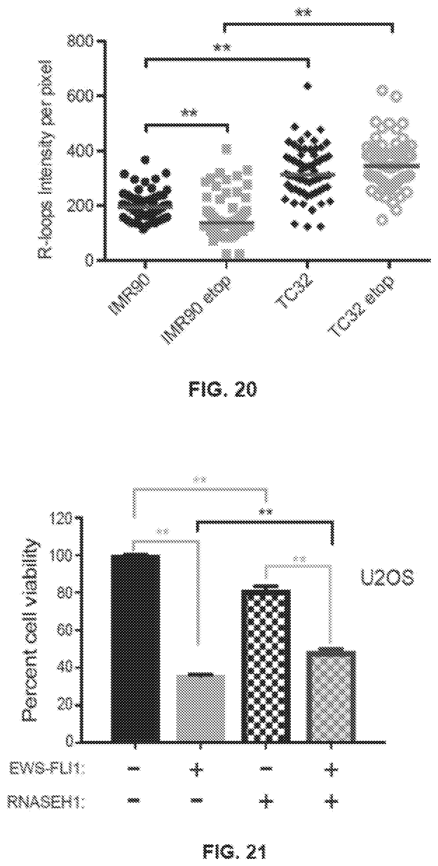

FIGS. 19A-19D present immunofluorescence images of cells stained with DAPI for the nucleus. FIGS. 19E-19H present immunofluorescence images of cells stained with S9.6 for the R-loops. FIGS. 19I-19L present immunofluorescence images of cells stained with nucleolin for the nucleoli. FIGS. 19M-19P present immunofluorescence images of cells that merges the staining from DAPI (nucleus), S9.6 (R-loops) and nucleolin (nucleoli). Scale bar (white line) provided in the bottom right represents 25 μm. FIG. 20 is a graphical representation of the quantification of nucleoplasmic R-loops in the immunofluorescence images (˜80 nuclei per set) that demonstrated clear increase in overall R-loop intensity in EwS cells. Nucleolin signal was used to subtract areas of R-loops present in the nucleoli. S9.6 signal was observed in both nucleoplasm and nucleoli (consistent with published data) and found to be significantly higher in TC32. The fold difference in EwS R-loops by this method was not as high as that obtained by the dot blot method. This difference in magnitude between the two data (FIG. 17A vs FIG. 20) can be ascribed to the inherent differences in the technology used to detect the R-loops as well as the specificity of detection (dot blot methods extracts all R-loops including nucleolar, whereas only the nucleoplasmic regions are evaluated by immunofluorescence).

If EWS-FLI1 driven R-loop accumulation has an adverse pathological effect, then this may explain the severe toxicity observed in heterologous cell systems upon EWS-FLI1 introduction. Overexpression of RNASEH1 would cause cells to be more amenable to EWS-FLI1 expression. This hypothesis was tested in U2OS cells expressing EWS-FLI1, RNASEH1 or both. FIG. 21 is a graphical representation of the quantification of cell viability of U2OS cells measured using Celltiter-glo after 48 hours of transfection with EWS-FLI1 or RNASEH1 or both and error bars represent SE from 8 independent transfections. #,*P<0.05; ##,**P<0.005, bar graphs display means with SE, n=4. Cell viability measured 48 hours post-transfection indicated a partial rescue in EWS-FLI1 mediated toxicity upon overexpression of RNASEH1.

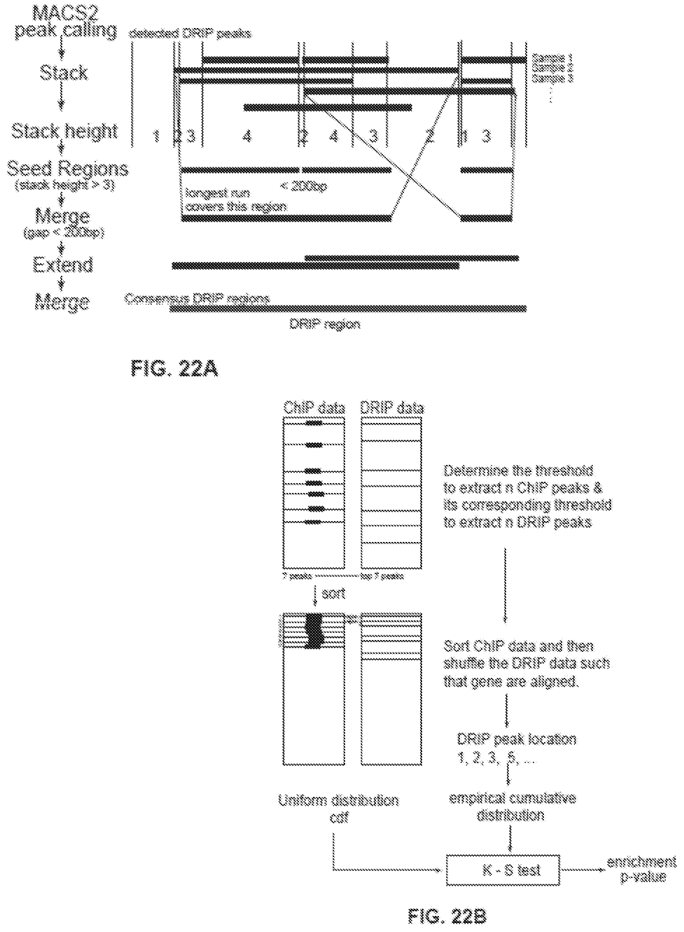

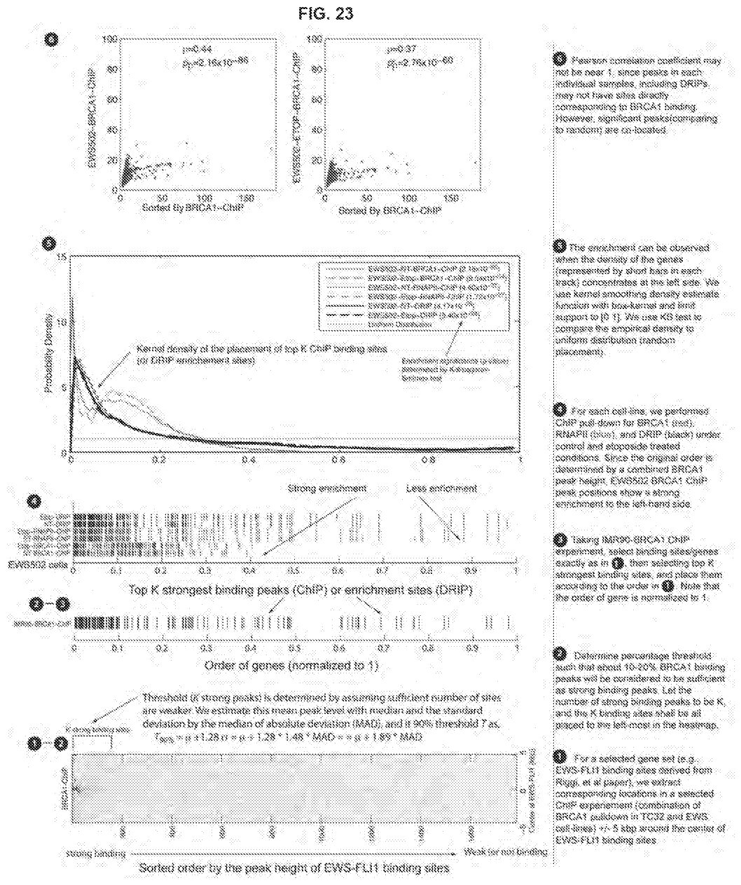

To further characterize the R-loops present in EwS, we conducted high-throughput sequencing and bioinformatics analysis of genomic DNA:RNA hybrids by immunoprecipitation (DRIP-Seq)18 of four EwS and both control cell lines with or without exposure to etoposide for 6 hours (FIGS. 22A-22B and 23A-23E). FIG. 22A is a flow chart of the algorithm used to determine consensus DRIP regions for each sample. Algorithm is detailed in the Methods section. FIG. 22B is a flow diagram of methodology designed to evaluate statistical significance of correlation between DRIP-Seq, ChIP-Seq (BRCA1 and EWS-FLI1) and expression data using Kolmogorov-Smirnov (KS) test (details in Methods and FIG. 23).

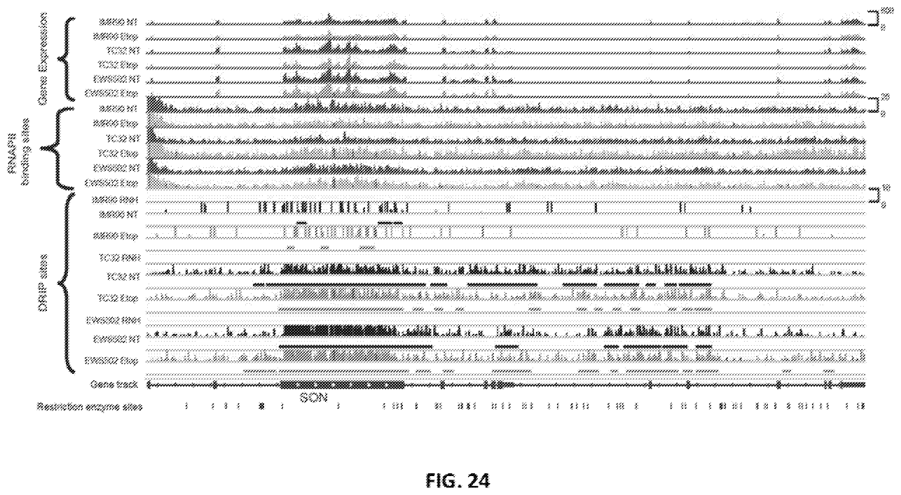

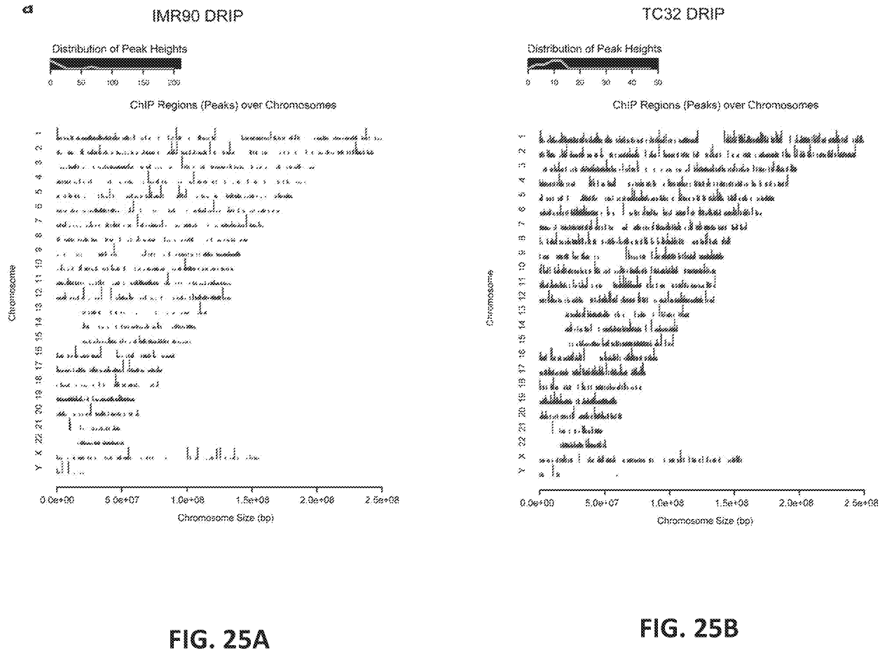

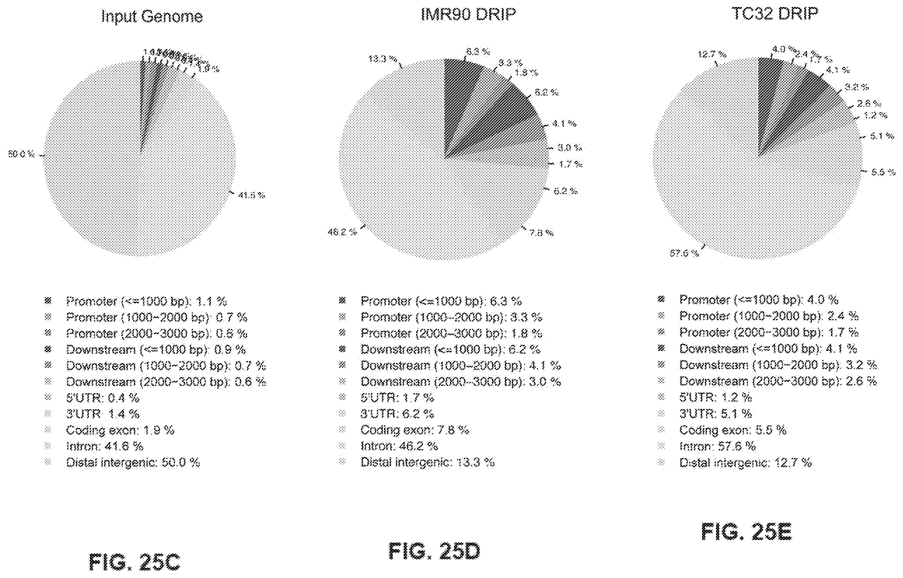

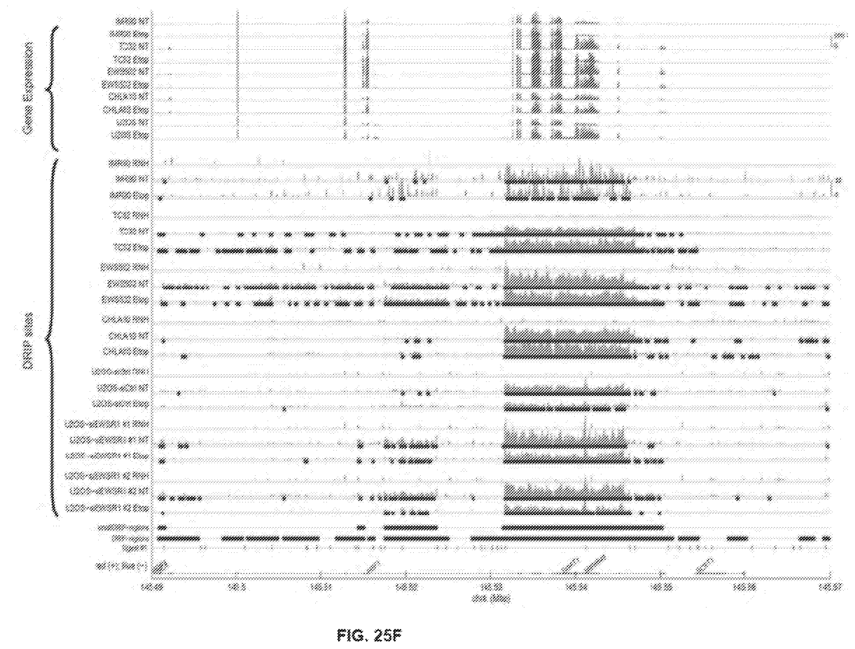

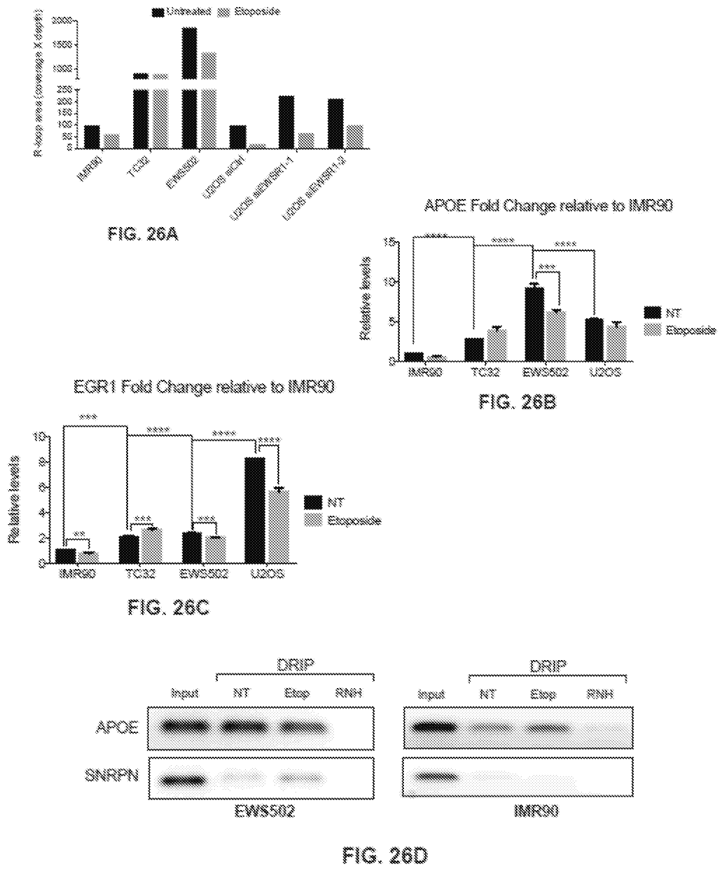

FIG. 23 is a schematic illustration of the Kolmogorov-Smirnov Test. This illustration depicts the analysis pipeline used in the Kolmogorov-Smirnov test to determine significance of enrichment between different probability distributions. This test was used to compare gene expression data, RNAPII binding sites, DRIP loci and BRCA1 binding sites among different cell lines with and without exposure to damage. Harvested DNA treated with RNaseH was also included as a negative control. At the outset, extensive R-loops w ere found throughout the genome (both read coverage and depth of reads) in EWSR1 depleted cells compared to control, and more so in EwS (FIG. 24, FIGS. 25A-25F, and FIG. 26A).

FIG. 24 is a schematic representation of a 35 kb region surrounding the SON gene. All cell lines are presented as individual tracks. RNA-seq data is depicted as red tracks followed by RNAPII ChIP-seq data in blue and DRIP-Seq reads in black. Untreated (NT) or etoposide treated (Etop) samples are presented in darker or lighter colors respectively. RNaseH treated (RNH) controls for DRIPs are also represented along with identified coverage regions (black or grey bars). Restriction enzyme sites track refers to the expected fragment size after restriction enzyme digestion of the genome before sequencing. The height of each track on the Y-axis represents the read counts and is indicated at the right.

FIGS. 25A and 25B are chromosome wide maps of R-loop sites in IMR90 and TC32 untreated DRIP samples as determined by the MACS2 program. FIGS. 25C-25E are representative pie charts depicting the differences in enrichment of DRIP between IMR90 and the EwS cell line EWS502. Technical biases during IP and sequence alignment can be evaluated based on the Input genome. The analysis was performed using MACS2 program. FIG. 25F is a representation of an 8 Mbp region of chromosome 8 surrounding the HSF1/DGAT genes. RNA-seq reads for untreated (NT) and etoposidetreated (6 hours, Etop) that are presented followed by DRIP reads and identified coverage regions (dark blue bars) for control, EwS and U2OS cells with scrambled (siCtrl) or EWSR1 (siEWSR1) siRNA. RNH tracks represent the RNaseH treated samples and serve as negative controls. Digest#1 refers to the expected fragment size after restriction enzyme digestion of the genome before sequencing. Y-axis represents the read counts. Navy blue bars reflect the identified DRIP regions, with additional tracks towards the bottom representing either smallDRIP-regions being the seed DRIP region (smallest common region across all samples) or DRIP-regions to include the longest contiguous region detected around the seed regions.

Qualitatively, an increased propensity for RNAPII binding the same regions as R-loops compared to surrounding genomic regions was observed, indicating increased RNAPII stalling (FIG. 24), which was confirmed by RNAPII ChIP-qPCR at well-established R-loop sites (FIGS. 26B-26C). Again, R-loops decreased in the 6 hours following damage, but less so in EwS. DRIP-seq data was validated using independent DRIP-PCR (FIG. 26D). FIG. 26A is a graphical representation of the quantification of DRIP (coverage of DRIP region multiplied by reads in that region) across all the samples. Y-axis is graphed in logarithmic scale. FIGS. 26B-26C are graphical representations of the fold enrichment of quantitative-PCR (qPCR) product from ChIP experiments done with RNAPII antibody in control and EwS cell lines. The primers target regions within APOE gene and EGR1 both of which contain R-loops. Statistical significance is relative to IMR90 no treatment (NT) sample. ***P<0.0005, bar graphs depict means with SE, n=3. FIG. 26D is a photograph of representative agarose gel blots testing validity of DRIPs by PCR at a well-known DRIP site in the target gene APOE. SNRPN serves as a negative control. NT: Not treated; Etop: Etoposide treated (6 hours); RNH: RNaseH treated.

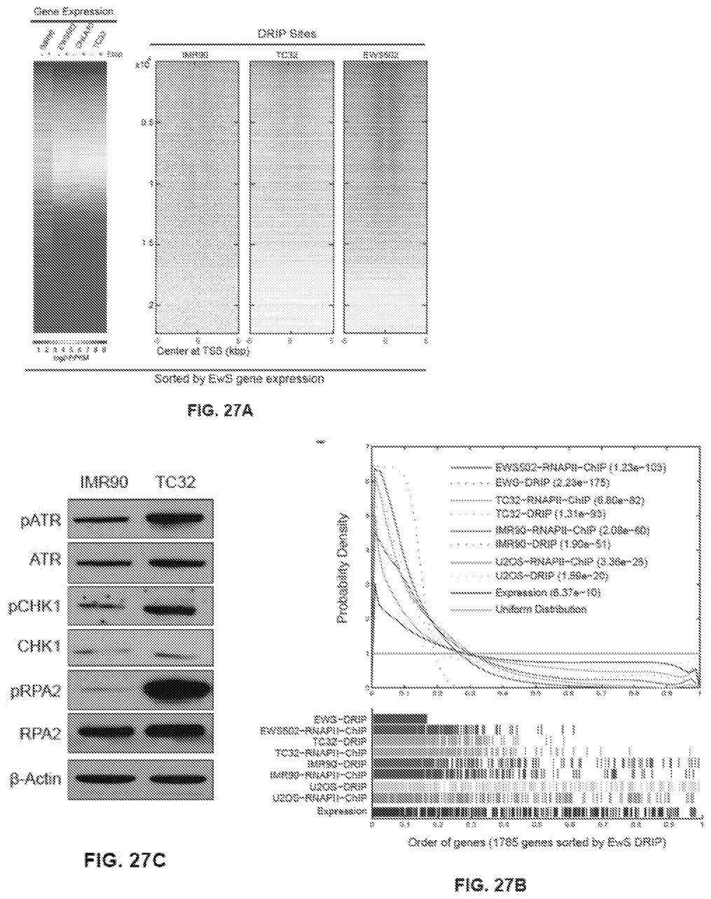

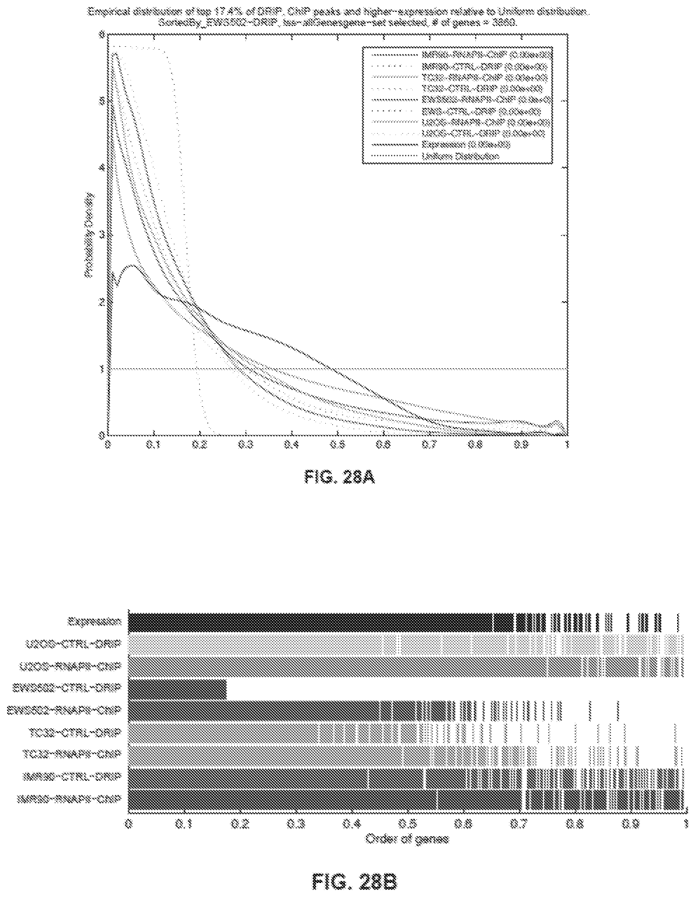

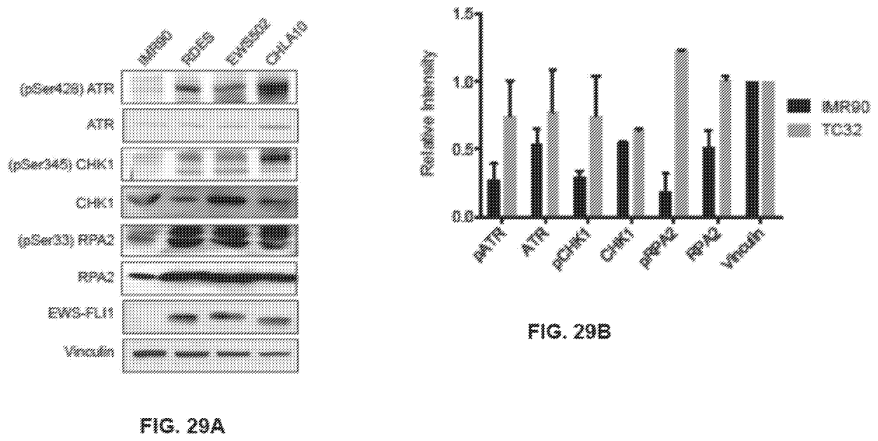

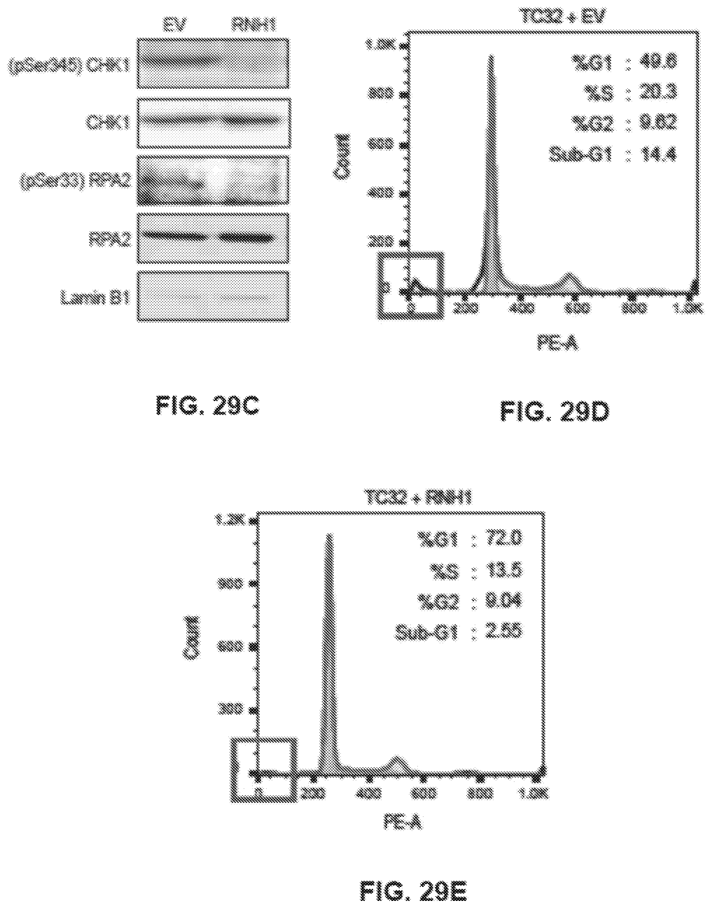

There were no specific differences in gene ontology associations among genes with R-loops in control versus EwS cells, suggesting that the R-loops in EwS were largely stochastic events resulting from altered transcription. A majority of the DRIPs mapped to intronic regions (FIGS. 25C-25E), with subtle changes to proportions in EwS cells. Comparing DRIP-Seq and RNA-Seq indicated a predominance of R-loops in highly expressed genic regions (FIG. 27A and FIGS. 28A-28B). FIG. 27A is a representative heatmap of genes (whole genome) centered around transcription start site (TSS) ordered by average expression of EwS cells. Profiles show a correlation between expression and presence of R-loops. EWS-FLI1 binding sites were extracted to determine co-occurrence with R-loops. Very strong enrichment was found between EWS-FLI1 binding and DNA:RNA hybrids (FIG. 27B), especially in the strongly expressed genes (top 16%). FIG. 27B is a probability density graph plotted with a Gaussian smoothing kernel of the distribution of DRIP peaks and EWS-FLI1 ChIP peaks at EWS-FLI1 bound genes relative to uniform distribution. The KS plot demonstrated enrichment of DRIP regions with gene expression at EWS-FLI1 binding sites. Number of genes represented=281 (top 16%). P-values depicting significance of enrichment for each sample is indicated in the inset boxes. FIG. 27C is a photograph of immunoblots showing increased levels of phospho-ATR, phospho-CHK1 and phospho-RPA2 in TC32 compared to IMR90.

FIG. 28A is a probability density plot of the distribution of DRIP peaks and expression profile at the corresponding peaks across all genes relative to uniform distribution. The KS plot indicated significant correlation (deviated from uniform distribution by Kolmogorov-Smirnov test) between actively transcribed genes and DRIP loci. There was also enrichment with RNAPII binding sites within this gene set. In FIG. 28B, the data was sorted by DRIP sites in EWS502. Number of genes used in analysis=3860 (top 17%). P-values depicting significance of enrichment for each sample is indicated in the inset boxes.

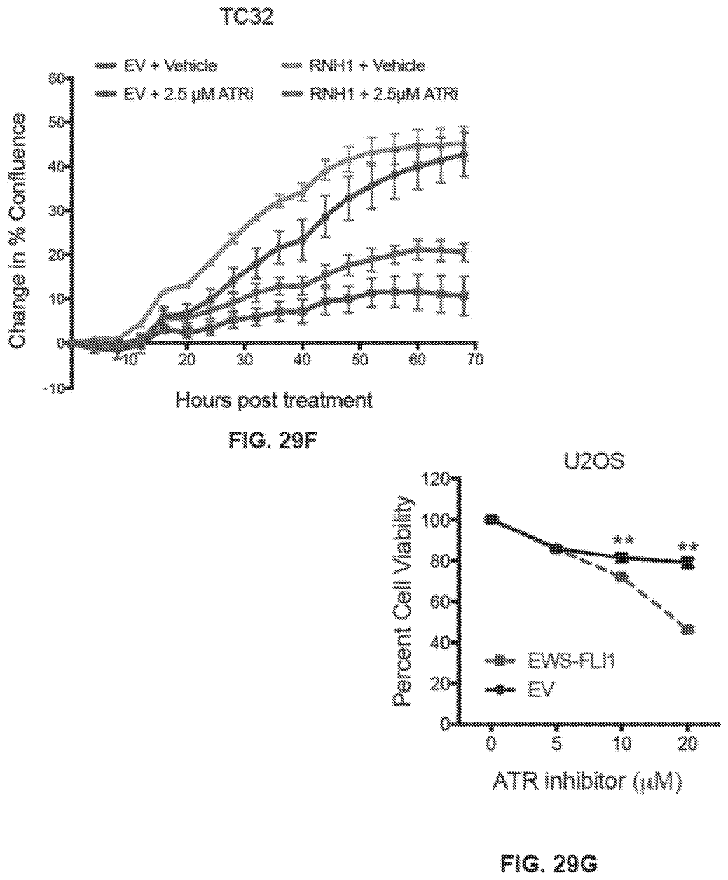

Unresolved R-loops are deleterious to the cell as they potentially block replication machinery progression and result in stalled or collapsed replication forks. We therefore hypothesized that EwS cells would display increased replication stress. Analysis of well-known markers of replication stress indicated elevated basal levels of activated (phosphorylated) ATR, CHK1 and RPA2 in EwS (FIG. 27C and FIGS. 29A and 29B). FIG. 29A is a photograph of immunoblots depicting a panel of EwS cell lysates evaluated for proteins that are activated (phosphorylated) in response to replication stress (ATR/CHK1/RPA2). Activation of this pathway was measured under basal conditions (no drug treatment). FIG. 29B is a graphical representation of the quantification of the immunoblots from two independent experiments of FIG. 29A.

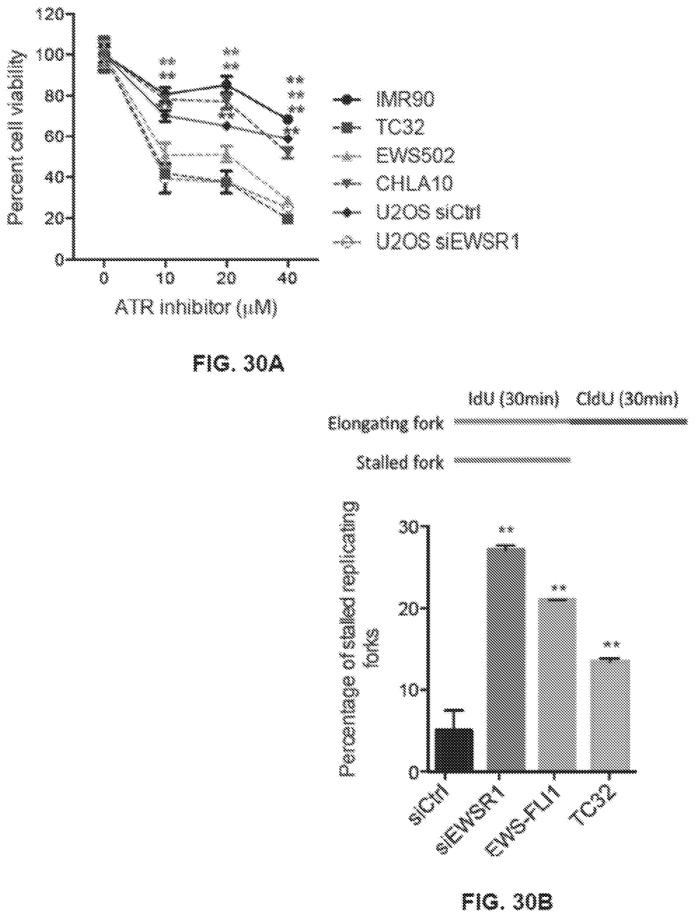

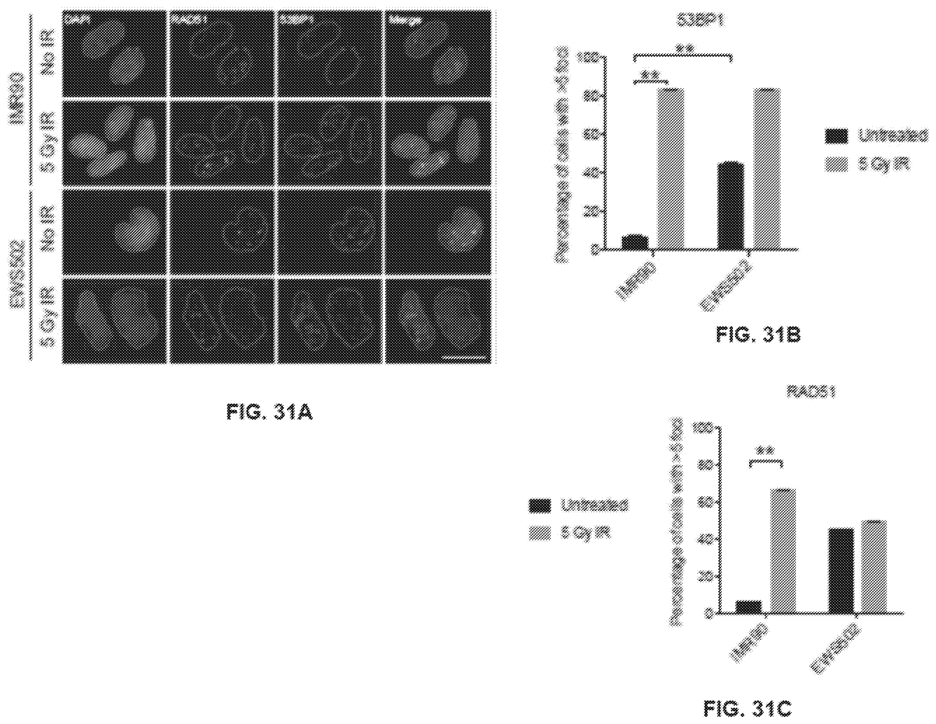

Overexpression of RNASEH1 was able to suppress the activation of this pathway (FIG. 29C) confirming the direct involvement of R-loop accumulation in augmenting replication stress. RNASEH1 overexpression also reduced the number of apoptotic TC32 cells (sub-G1 population, FIGS. 29D-29E) and improved cell proliferation rate (FIG. 29F). FIG. 29C is a photograph of representative immunoblots evaluating decrease in the ATR kinase pathway activation upon overexpression of RNASEH1 in TC32 cells. FIGS. 29D and 29E are representations of DNA content analysis using flow cytometry on TC32 cells with and without RNASEH1 overexpression respectivly. Percentages of cells in each phase of the cell cycle are indicated in the top right of the graph. Sub-G1 is highlighted by a red box. FIG. 29F is a graphical representation of the cell viability plot displaying proliferation rate of TC32 cells with and without RNASEH1 overexpression over time after treatment with either vehicle or an ATR inhibitor (VE-821, ATRi). The Y-axis represents change in confluence from the starting point: each cell line's initial confluence was normalized to zero and the difference in confluence at subsequent time points was plotted. EV: Empty vector. The data was collected from Incucyte Live Cell Imaging system. EV: Empty vector. With increased replication stress, EwS cell lines, as well as U2OS cells with EWSR1 knockdown or EWS-FLI1 expression, were significantly more sensitive to ATR inhibition (FIG. 29G and FIG. 30A). FIG. 29G is a graphical representation of the cell viability plot based on confluence measurements by IncuCyte showing increased sensitivity to ATR inhibitor in U2OS cells transfected with EWS-FLI1 compared to empty vector (EV) transfected cells. **P<0.005, line graph represents means with SE, n=3. EwS sensitivity to ATR inhibition was reduced by RNASEH1 overexpression (FIG. 29F).

FIG. 30A is a graphical representation of the cell viability response to ATR inhibitor (VE-821) in control, EwS and EWSR1 depleted U2OS cells. Finally, using DNA fiber spreading technique, there was a loss of EWSR1 function, either by silencing in U2OS or expression of EWS-FLI1 (U20S/EwS cell lines) that resulted in an increased number of stalled forks. FIG. 30B is a graphical representation of the replication stress as quantified by evaluating number of stalled replication forks (Schematic, IdU label only) using DNA fiber spread method. *P<0.05; **P<0.005, the colors of the * match that used for the cell line in the cytotoxicity plots and was tested against IMR90. In case of U2OS siEWSR1, P-value was calculated relative to U2OS siCtrl. Bar and line graphs display means with SE, n=4. In conclusion, accumulation of R-loops and replication stress in the absence of EWSR1 function, either by knockdown or due to interference from EWS-FLI1. Loss of EWSR1 function results in impaired recombination. Another consequence of accumulated R-loops is increased DNA damage and elevated HR. FIG. 31A is a set of immunofluorescence images taken at 40× magnification to detect number of DSBs (53BP1 foci) and IR-induced RAD51 foci. Scale bar (white line) provided at the bottom right represents 25 μm. FIG. 31B is a graphical representation of the quantification of number of cells with >5 53BP1 foci and FIG. 31C is a graphical representation of the quantification of number of cells with >5 RAD51 foci. n=3 sets of 80 nuclei each. EwS cell lines exhibited high levels of DNA damage as measured by 53BP1 foci (FIGS. 31A and 31B) compared to IMR90. EwS cells were evaluated for hyper-activation of HR. There was a surprising lack of IR-induced RAD51 foci (FIGS. 31A and 31C). Interestingly, basal levels of RAD51 foci were higher in EwS than with IMR90 cells (FIG. 31C), though this may reflect the increased replication stress.

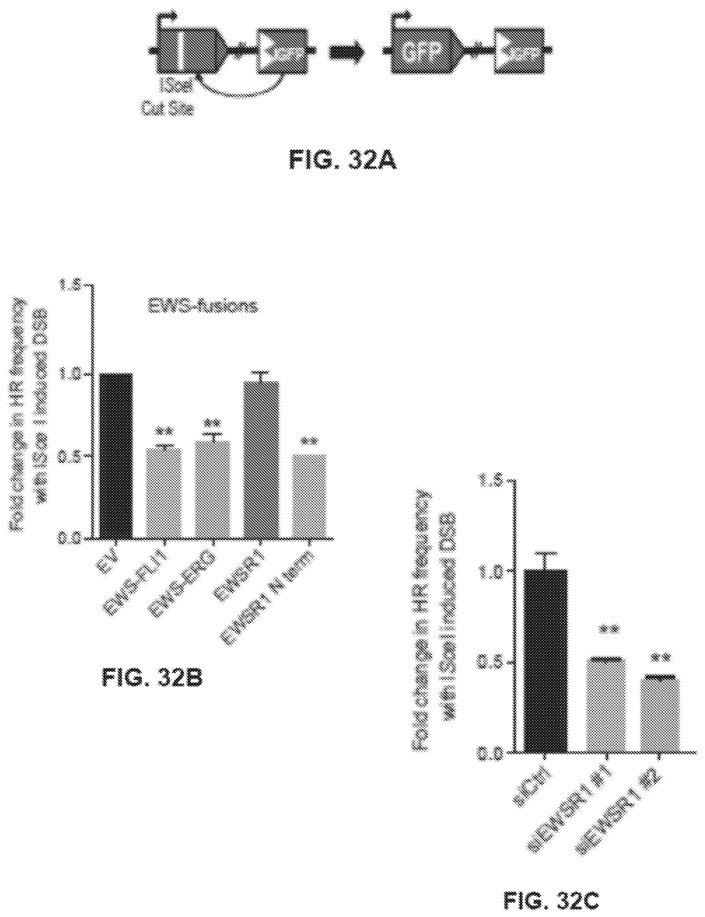

To confirm the induced HR defect, a well-established direct-repeat GFP assay was used (DR-GFP) integrated into U2OS cells. FIG. 32A is a schematic illustration of the DR-GFP construct integrated into U2OS cells used to measure HR. FIG. 32B is a graphical representation of HR frequency, measured as number of GFP positive cells (flow cytometry) with transient transfection of EWS-FLI1, EWS-ERG (two most common translocations in EwS), EWSR1-full length (EWSR1) and EWSR1 N terminal domain only (EWSR1 N term) relative to empty vector (EV) DR-GFP U2OS cells. DR-GFP U2OS cells transfected with EWS-FLI1 displayed significantly reduced HR capacity (FIG. 32B). Similar results were obtained with the next most common EWSR1-translocation (EWS-ERG; found in ˜10% of patients) associated with EwS, suggesting that the observed phenotype is not specific to EWS-FLI1. EWS-FLI1 is known to bind EWSR1 through its N terminal domain and impede its function. Then, the HR suppression was evaluated as an effect of a loss of EWSR1 functionality. FIG. 32C is a graphical representation of the results from a DR-GFP assay used to evaluate effect of silencing EWSR1 with two independent siRNA on HR. As suspected, either expression of the EWSR1 N-terminal domain alone (FIG. 32B) or two independent siRNA against EWSR1 (FIG. 32C) also resulted in a reduction of HR frequency.

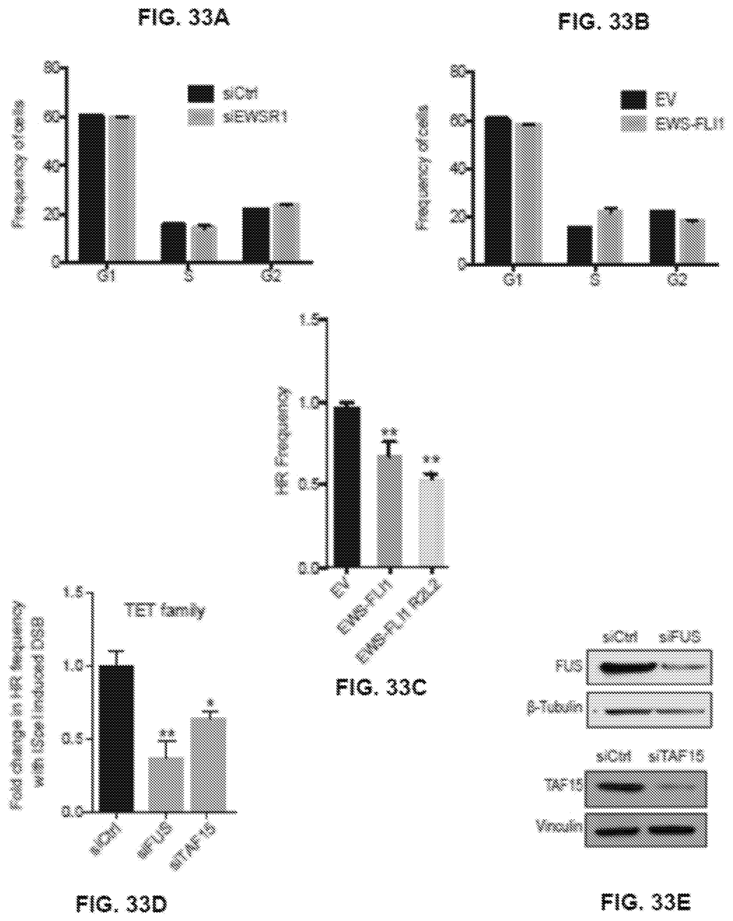

Cell cycle differences upon EWSR1 knockdown or EWS-FLI1 expression did not account for the differences in HR frequency (FIGS. 33A and 33B). FIGS. 33A-33B are graphical representations of quantification of percentage of cells in the different phases of cell cycle with either knockdown of EWSR1 (FIG. 33A) or expression of EWS-FLI1 (FIG. 33B). The DNA binding mutant of EWS-FLI1 (R2L2) also demonstrated suppression of HR, confirming that this phenomenon is independent of its DNA binding activity (FIG. 33C). FIG. 33C is a graphical representation of quantification of HR frequency following expression of either EWS-FLI1 or DNA binding mutant EWS-FLI1 R2L2. Bar graphs represent fold change relative to empty vector (EV) transfected DR-GFP U2OS cells. Schematic of EWS-FLI1 R2L2 was presented in FIGS. 17J-17K. Since all members of the TET family are known to interact with each other, FUS and TAF15 were depleted and this demonstrated that both proteins were also important for HR (FIGS. 33D and 33E). FIG. 33D is a graphical representation of quantification of HR frequency in DR-GFP U2OS cells transfected with either scrambled siRNA (siCtrl), FUS siRNA (siFUS) or TAF15 siRNA(siTAF15). FIG. 33E is a photograph of an immunoblot showing the levels of knockdown of FUS siRNA (siFUS) or TAF15 siRNA(siTAF15). *P<0.05, **P<0.005, bar graphs represent means with SE, n=3.

Given the similarity in phenotypes between EwS and BRCA1/2 mutant breast cancer (Table 7), BRCA1 function was evaluated in EwS.

Table 7. Comparison of EwS and BRCA-mutant cancers. The table enumerates characteristic hallmarks of BRCA1-mutant breast cancers and whether these phenotypes are similarly observed in EwS.

TABLE 7

| | BRCA1/2 mutant | Ewing |
|---|---|---|
| Phenotype | cancers | sarcoma |
| Sensitivity to DNA damaging agents | ✓ | ✓ |
| Sensitivity to PARP1 inhibitors | ✓ | ✓ |
| Hyperactivation of PARP1 | ✓ | ✓ |
| Impaired homologous recombination | ✓ | ✓ |
| Accumumation of R-loops | ✓ | ✓ |
| High expression of EZH2 | ✓ | ✓ |
| Affects alternative splicing | ✓ | ✓ |
| Genome Instability | ✓ | X |

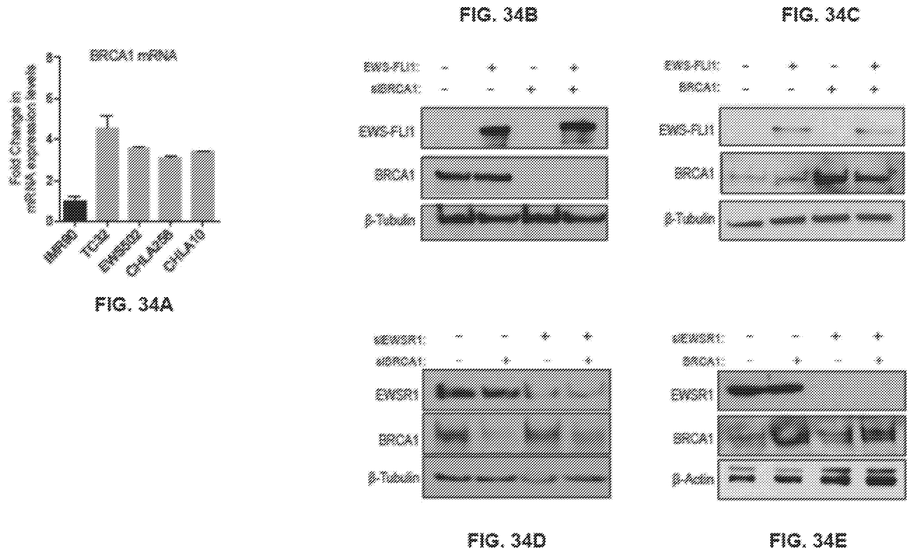

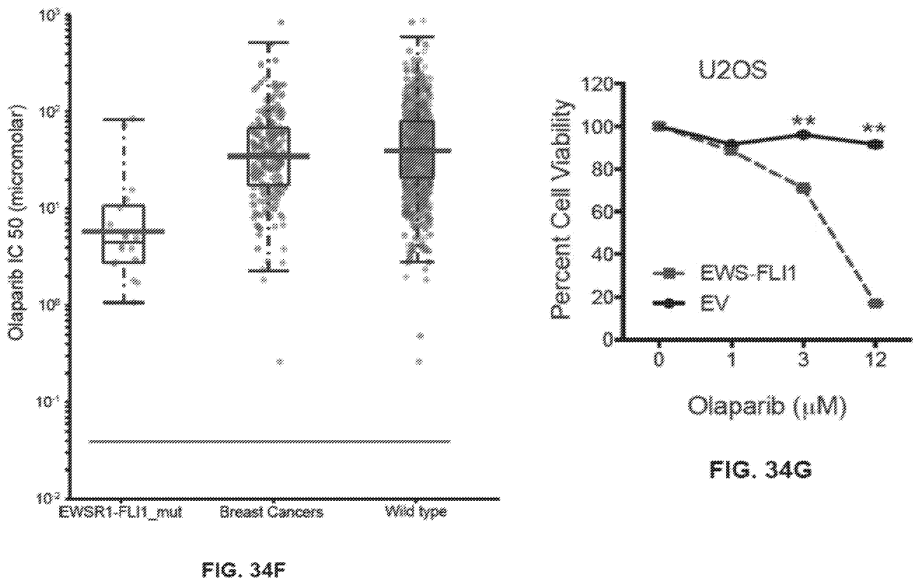

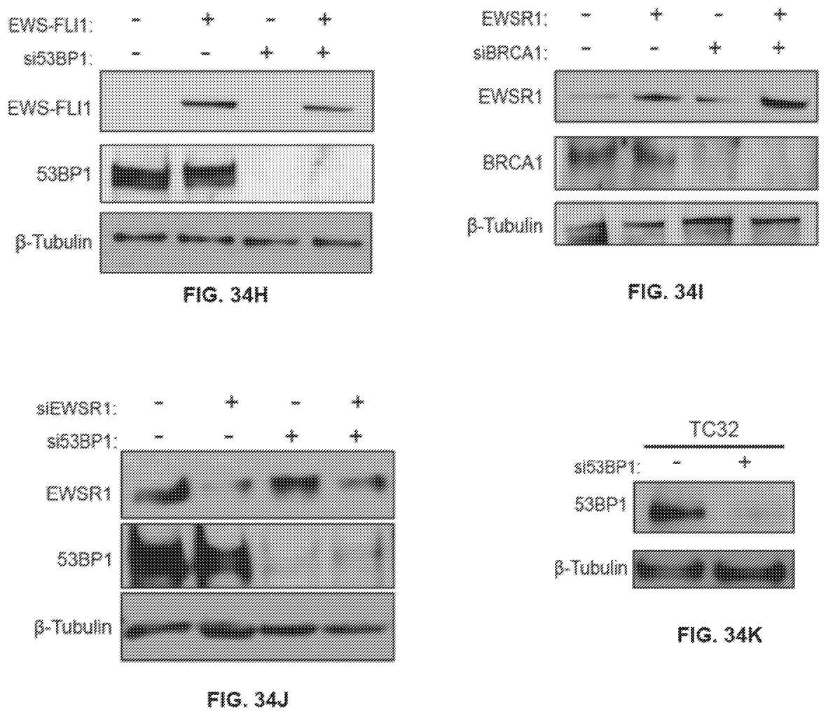

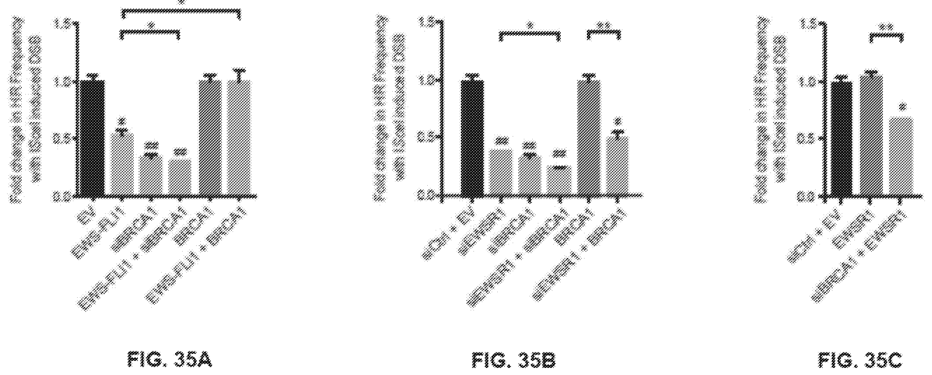

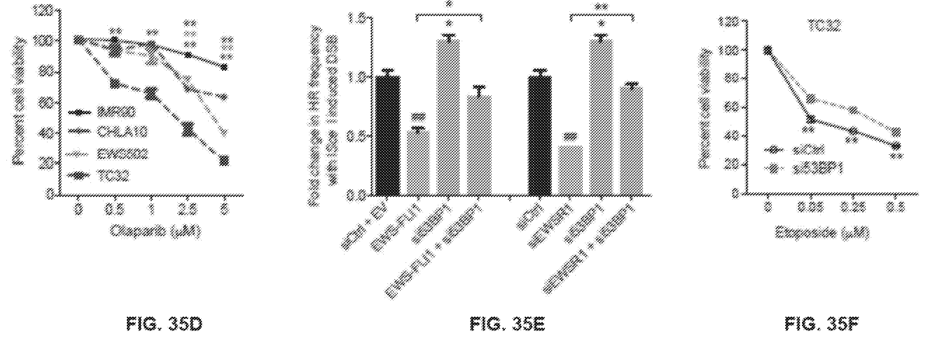

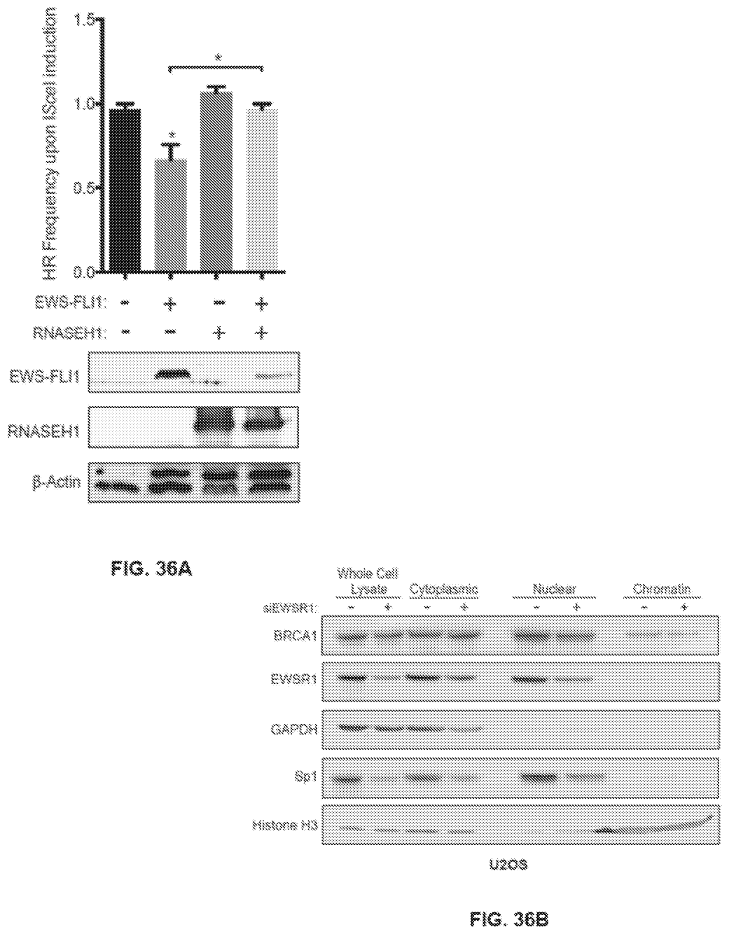

Interestingly, gene expression analysis comparing EwS and control cells identified significant enrichment for a breast cancer with BRCA1-mutated gene set (FIGS. 6C-6F). EwS cell lines were found to have robust expression of BRCA1 (FIG. 34A) with no known mutations. Alternatively, BRCA1 could be functionally unavailable for HR. In order to test this, BRCA1 was overexpressed and restoration in HR activity was observed in the context of EWS-FLI1 expression (FIG. 35A).

FIG. 34A is a graphical representation of RNA-seq data of BRCA1 transcript levels in EwS cell lines compared to IMR90. FIGS. 34B, 34C, 34D, and 34E are photographs of immunoblots demonstrating level of transfection of various siRNA and expression constructs used in FIGS. 35A-35C. EwS cells are acutely sensitive to PARP1 inhibitor, olaparib. FIG. 34F is a box-and-whisker plot depicting the IC50 levels of olaparib in EWS-FLI1 mutant versus just breast cancers or pan-cancer cell lines. The EWS-FLI1 mutant group contains 17 cell lines, breast cancers group contains 13 cell lines and the pan-cancer group contains 147 cell lines. FIG. 34G is a graphical representation of cell viability demonstrating the role of EWS-FLI1 in mediating exquisite sensitivity to olaparib in U2OS cells transfected either with the oncogene or empty vector (EV). FIGS. 34H, 34I, and 34J are photographs of immunoblots demonstrating level of transfection of various siRNA and expression constructs used in FIG. 35E. FIG. 34K is a photograph of an immunoblot depicting level of 53BP1 knockdown in TC32 for cell viability studies in FIG. 4l. **P<0.005, Line and bar graphs represent means with SE, n=4.

FIG. 35 A is a graphical representation of measurements of HR frequency in DR-GFP U2OS cells expressing EWS-FLI1 with either siRNA against BRCA1 or overexpression of BRCA1 (transfection efficiency demonstrated in FIGS. 34B-34E). Bars represent fold change in HR relative to empty vector (EV) transfected cells. FIGS. 34B-34E are photographs of immunoblots demonstrating level of transfection of various siRNA and expression constructs.

Intriguingly though, BRCA1 overexpression was unable to restore HR in combination with EWSR1 depletion (FIG. 35B). FIG. 35B is a graphical representation of the fold change in HR frequency in DR-GFP U2OS cells with EWSR1 depletion in combination with either siRNA against BRCA1 or BRCA1 overexpression. Fold change was calculated relative to HR frequency measured in cells transfected with siCtrl+EV (transfection efficiency demonstrated in FIGS. 34B-34E). Overexpression of EWSR1 in the context of BRCA1 knockdown was also unable to rescue the HR defect (FIG. 35C), indicating that EWSR1 and BRCA1 cooperate functionally for efficient HR. PARP1 inhibition leads to accumulation of substrates for the BRCA1-dependent HR pathway and in cells lacking BRCA1, the use of PARP1 inhibitors leads to synthetic lethality. The aforementioned BRCA1 unavailability and subsequent HR deficiency provides a molecular basis for the exquisite sensitivity observed for EwS to PARP1 inhibitors such as Olaparib (FIG. 35D and FIGS. 34F and 34G) as well as other damages that can be repaired by HR, including etoposide induced DNA breaks. FIG. 35C is a graphical representation of the fold change in HR frequency in DR-GFP U2OS cells with EWSR1 overexpression in the presence of BRCA1 knockdown relative to siCtrl+EV transfected cells (transfection efficiency demonstrated in FIGS. 34H-34J). FIG. 35D is a cytotoxicity plot of IMR90 and EwS cells exposed to increasing doses of Olaparib. Cell viability was measured 72 hours post treatment. Therefore, to confirm functional deficiency of BRCA1 in EwS, we evaluated whether loss of 53BP1 could similarly rescue the HR defect in the EwS context. 53BP1 depletion restored HR in DR-GFP U2OS cells with either EWSR1 knockdown or EWS-FLI1 expression (FIG. 35E). FIG. 35E is a graphical representation of the depletion of 53BP1 partially rescued the HR defect conferred by either EWS-FLI1 expression or EWSR1 knockdown in the DR-GFP assay (transfection efficiency demonstrated in FIGS. 34H-34J). Knockdown of 53BP1 also resulted in a moderate rescue of sensitivity to etoposide in EwS (FIG. 35F). FIG. 35F is a graphical representation of the 53BP1 knockdown improved EwS (TC32) survival in response to damage (transfection efficiency demonstrated in FIG. 34K). The symbol # indicates statistical significance relative to control (black bar) #,*P<0.05; ##,**P<0.005 the colors of the * match that used for the cell line in the multi-cell line cytotoxicity plots and was tested against control. Bar and line graphs display means with SE, n=3. It has been shown that secondary mutations in 53BP1 can circumvent the need for BRCA1 in HR, partially restoring HR and providing some resistance to various chemotherapeutics. Taken together, EwS phenocopies BRCA1-deficient tumors (Table 7 and FIGS. 6C to 6F) and illustrates secondary mutations in 53BP1 as a potential EwS chemoresistance mechanism.

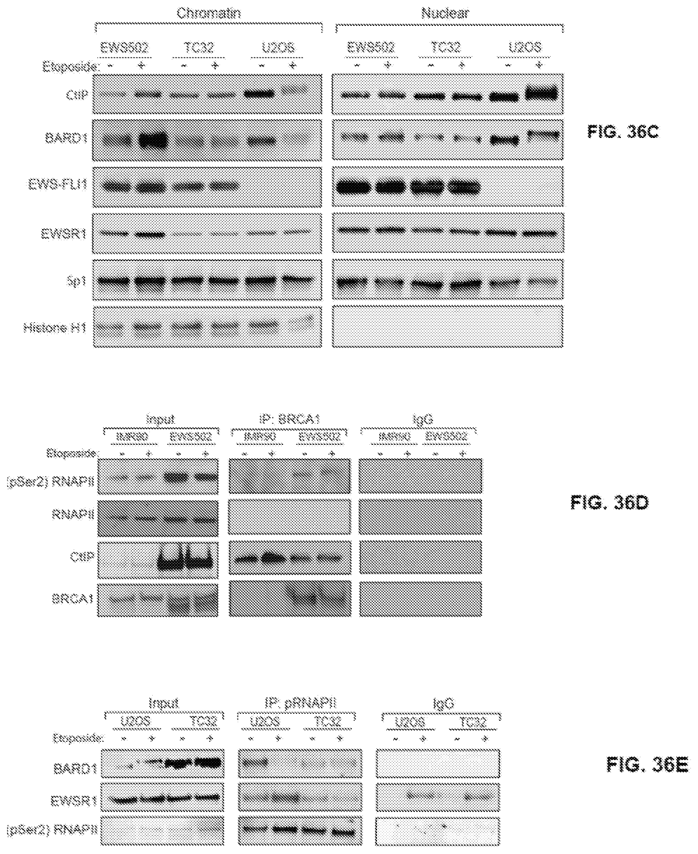

BRCA1 is sequestered with transcription complexes. R-loops cause replication stress and consequently induce HR at stalled replication forks. Co-expression of RNASEH1 rescued the HR suppression by EWS-FLI1 at a double strand break (DSB, FIG. 36A), suggesting that the accumulation of R-loops across the genome might be responsible for the impaired HR capability at DR-GFP. FIG. 36A is a graphical representation of quantification of HR frequency in DR-GFP U2OS cells transfected with EWS-FLI1, RNASEH1 or both. Overexpression RNASEH1 in the context of EWS-FLI1 restored HR. All values were normalized to the frequency of GFP positive cells observed in empty vector transfected cells. Representative immunoblots indicating transfection efficiency are presented below the graph. FIG. 36B is a photograph of immunoblots of whole cell lysates and various subcellular fractions from U2OS cells with and without EWSR1 depletion. Data indicated lack of change in BRCA1 levels with EWSR1 knockdown. Loading controls for the various fractions (GAPDH for cytoplasm, Sp1 for nuclear and Histone H3 for chromatin) as well as level of knockdown (EWSR1) are also displayed. FIG. 36C is a photograph of immunoblots of chromatin and nuclear fractions of EwS and U2OS cell lines with and without etoposide treatment (2 hours). FIG. 36D is a photograph of immunoblots of IMR90 and EWS502 nuclear lysates with and without exposure to etoposide (2 hours). The left panel indicates 10% of the input used for IP. BRCA1 antibody was used for IP in the middle panel and the rightmost panel indicates specificity of interaction against IgG IP. FIG. 36E is a photograph of immunoblots following co-immunoprecipitation with phosphorylated (Ser5) RNAPII in nuclear lysates from U2OS and TC32 with and without exposure to etoposide (2 hours). *P<0.05, bar graph represents means with SE, n=4.

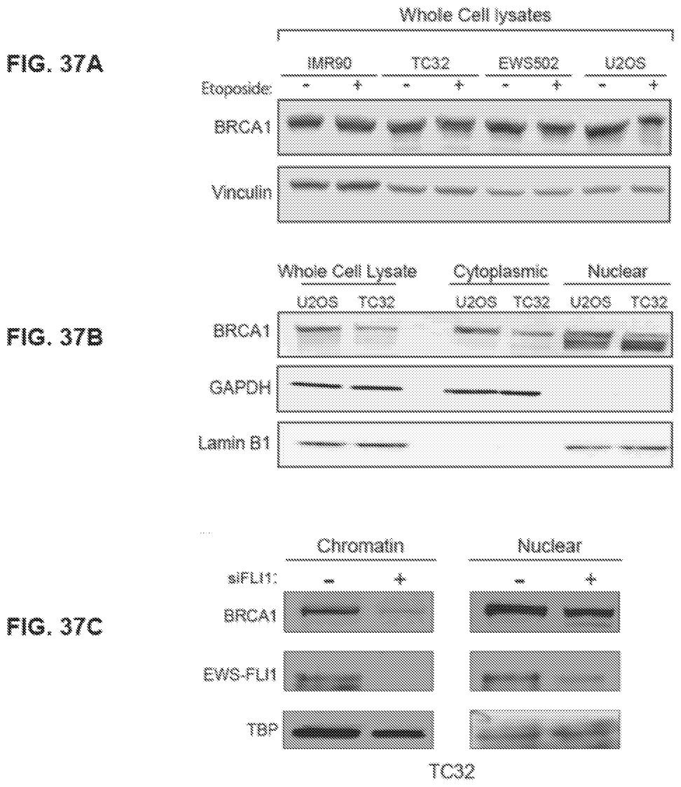

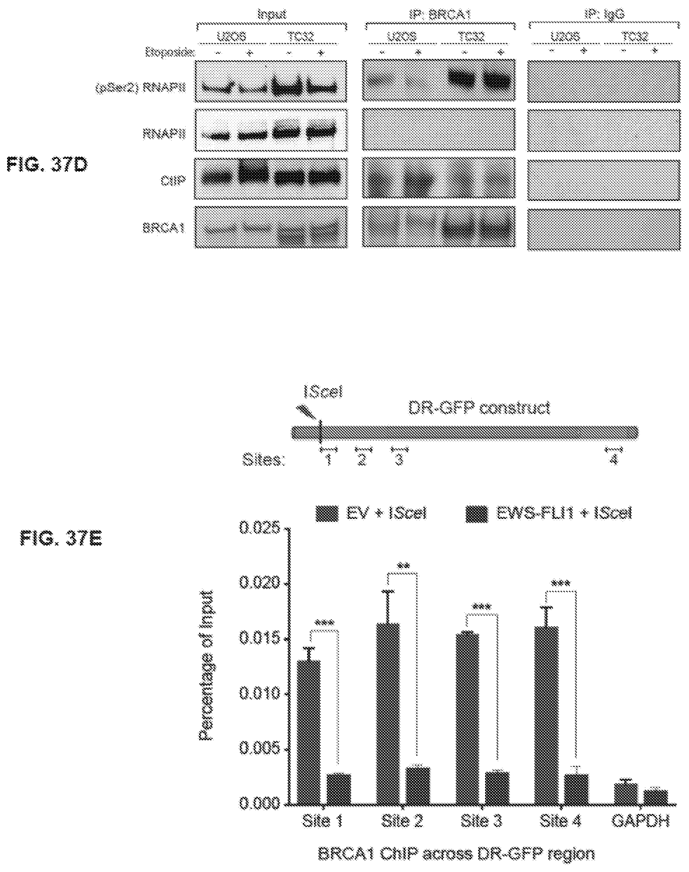

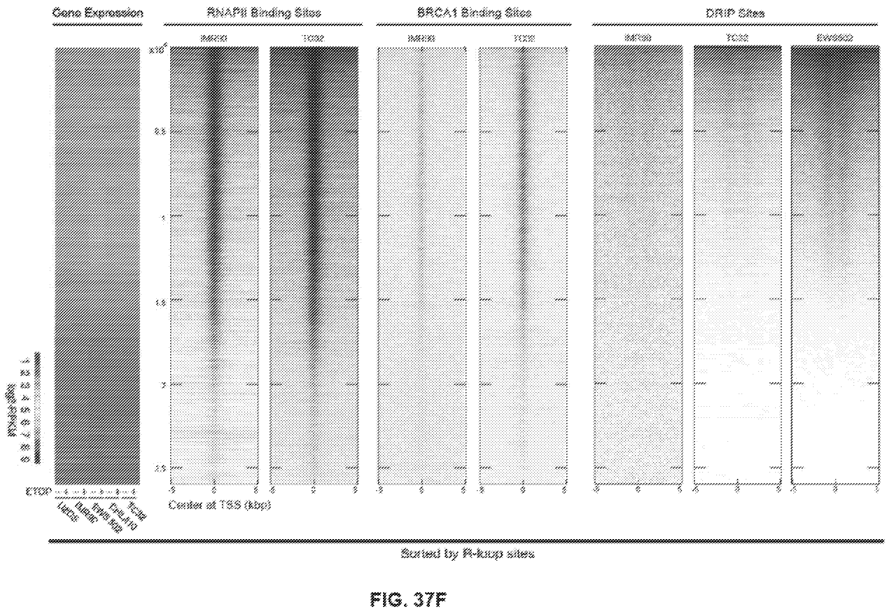

BRCA1 is associated with the elongating transcription complex and has been associated with sites of R-loops. It may be possible to alter BRCA1 function in EwS, being sequestered at transcription complexes and unable to respond to damage. It is important to note that overall levels of BRCA1 protein were similar between EwS and control cells (FIG. 37A).

FIG. 37A is a photograph of immunoblots showing equivalent levels of BRCA1 in whole cell lysates from control and EwS cells with and without etoposide treatment (2 hours). However, it appears that there is a redistribution of BRCA1 in subcellular fractionations of U2OS versus TC32 lysates with more nuclear BRCA1 in the EwS cells (FIG. 37B). FIG. 37B is a photograph of immunoblots showing the differences in BRCA1 redistribution in subcellular fractions of U2OS or TC32 cells. GAPDH and Lamin B1 were used as loading controls for the cytoplasmic and nuclear fractions respectively.

Interestingly, further evaluation of subcellular fractionation of EwS lysates exposed enrichment in chromatin-bound BRCA1 (FIG. 37C) that was substantially reduced upon knockdown of EWS-FLI1. Loss of EWSR1 did not result in a similar sequestration of BRCA1 in the chromatin fraction (FIG. 36B). FIG. 37C is a photograph of immunoblots showing BRCA1 and FLI1 in the nuclear and chromatin fractions of TC32 with and without EWS-FLI1 knockdown.

To further ascertain the DNA damage-dependent redistribution in repair proteins, we examined subcellular fractions of EwS and U2OS cells exposed to etoposide (FIG. 36C). U2OS displayed a dramatic decrease in BARD1 and CtIP post damage in the chromatin fraction with a concomitant increase in the soluble nuclear fraction along with a change in migration rate indicating post-translational modifications. This alteration was lacking in both EwS cell lines tested suggesting a lack of damage-dependent activation of these key proteins. To better understand the relationships between phosphorylated RNAPII and BRCA1 in control and EwS cells, their interaction was examined using co-immunoprecipitation (co-IP) experiments (FIG. 37D) using endogenous proteins from nuclear lysates. FIG. 37D is a photograph of immunoblots showing immunoprecipitations with antibodies against BRCA1 or IgG in nuclear lysates of U2OS and TC32 with or without exposure to etoposide (2 hours). Input represents 10% of the amount used for immunoprecipitation. BRCA1 is known to associate preferentially with the hyperphosphorylated RNAPII in undamaged cells and as previously noted, EwS displayed high levels of phospho-RNAPII (FIG. 11A). BRCA1 immunoprecipitated phospho-RNAPII in our experiment as well, however, correlating with the high levels of phospho-RNAPII, an increased proportion of BRCA1 was involved in this interaction in the EwS cells. This result highlights the redistribution of BRCA1 to transcription complexes compared to control cells. Further, this interaction has been shown to diminish following damage, as was confirmed in our control cell lines (FIG. 37D). However, there was still considerable interaction between BRCA1 and phospho-RNAPII post-damage in the EwS cells. Total RNAPII was detected in the input, but not in the IP samples since the antibody preferentially recognizes the hypophosphorylated form of RNAPII. Subsequent to release from the transcription machinery, BRCA1/BARD1 association with CtIP increases to promote 53BP1 removal and DSB repair via HR. The BRCA1-CtIP interaction was examined, which increased in response to damage in our control cells. However, this increase in damaged-induced interaction was clearly abolished in the EwS cells. The above interactions were confirmed in endogenous proteins from independent control and EwS cell lines (FIG. 36D). Interestingly, pull-down with phospho-RNAPII indicated a decrease in interaction with BARD1 and a concomitant increase in interaction with EWSR1 upon damage in U2OS (FIG. 36E). Again, these damage-dependent alterations were not observed in EwS. In order to confirm the lack of damage-dependent BRCA1 interaction with repair factors, BRCA1-ChIP was conducted in U2OS cells after ISceI induction of a DSB at the DR-GFP site. The data clearly revealed a lack of BRCA1 recruitment to the break site in cells transfected with EWS-FLI1 compared to vector transfected controls (FIG. 37E). FIG. 37E is a graphical representation of the relative abundance of BRCA1 occupancy in DR-GFP U2OS cells transfected with either empty vector (EV, grey bars) or EWS-FLI1 (red bars) at various sites near the IScel induced DSB. GAPDH was used as a negative control. The schematic above the bar graph depicts the positions of the primers used for qPCR relative to the ISceI break site. FIG. 37F is a heatmap representation of correlation between gene expression, RNAPII and BRCA1 binding sites, and DRIP locations across the entire genome of IMR90, TC32 and EWS502 cells ordered by DRIP loci centered around the transcription start site (TSS). ***P<0.0005, bar graph displays means with SE, n=3. These data mechanistically confirm that the HR deficiency in EwS is due to sequestration of BRCA1 in transcription complexes resulting in its functional unavailability for DSB repair. In order to perform an in-depth analysis of BRCA1 association with R-loops sites, genome wide BRCA1 binding sites were obtained in our control and EwS cell lines with and without exposure to damage.

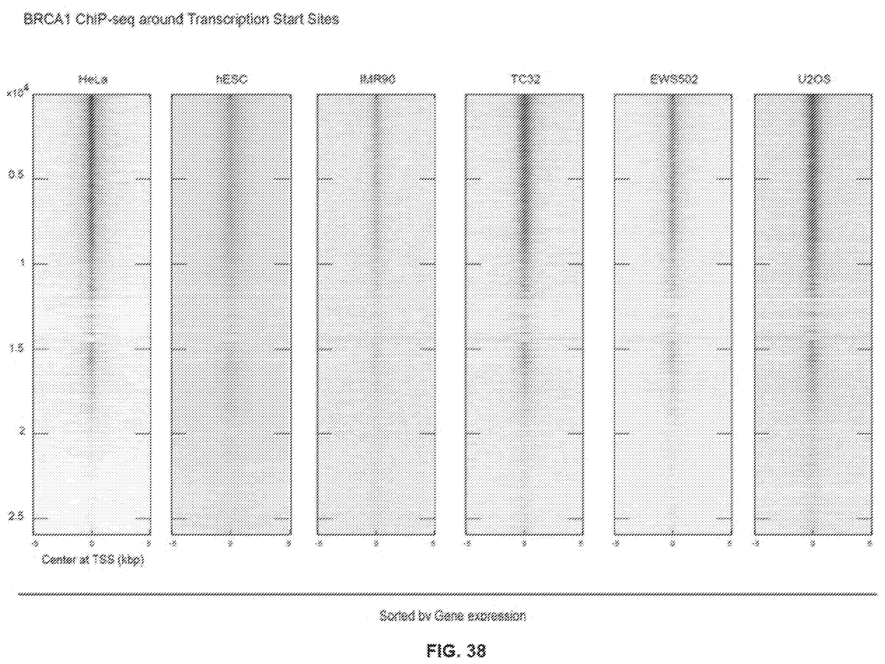

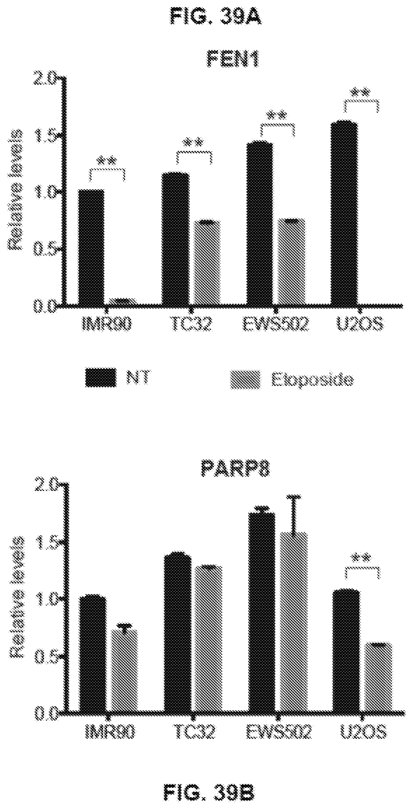

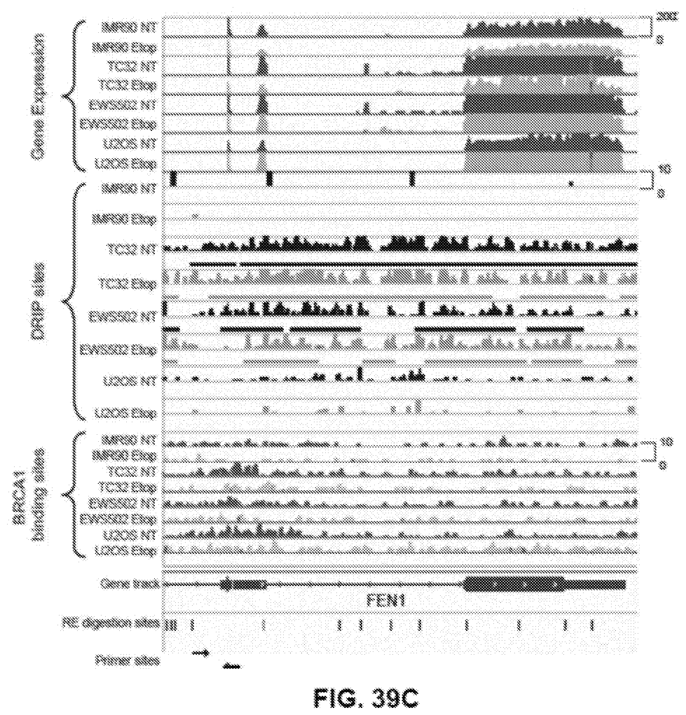

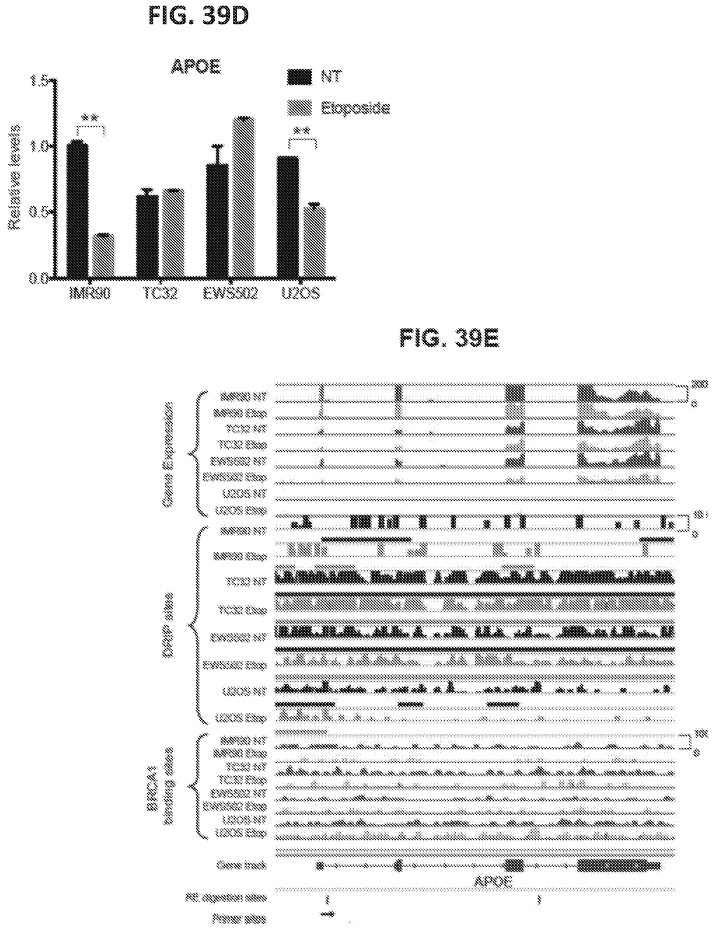

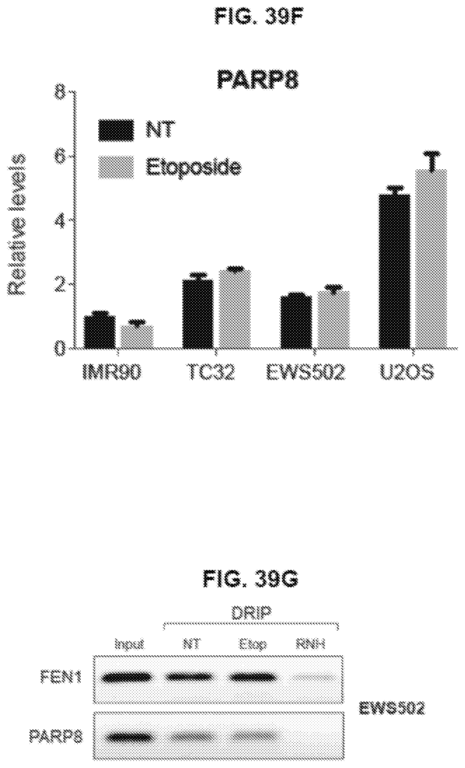

Comparison with BRCA1 ChIP data sets from ENCODE indicated a strong concordance of BRCA1 localization, confirming the quality of the chromatin immunoprecipitation (ChIP)-seq data. FIG. 38 is a heatmap showing a strong concordance between the ENCODE BRCA1 ChIP datasets from HeLa and human embryonic stem cells (hESC) and the ChIP data generated in-house (IMR90, TC32, EWS502 and U2OS). The data was sorted by gene expression across the entire genome and centered at the transcription start site (TSS).The data was validated with quantitative real-time PCR of ChIP DNA at two well-known BRCA1 binding sites (FIGS. 39A-39C). BRCA1 binding to these sites significantly decreased upon damage in IMR90 and U2OS cells, but the decrease was not as strong in the EwS cells. FIG. 39A is a graphical representation of results from real-time qPCR analysis of BRCA-ChIP samples processed from the different control and EwS cell lines with and without etoposide treatment, using primers within FEN1. FIG. 39B is a diagrammatic representation of gene expression (red tracks), R-loop sites (black and grey tracks) and BRCA1 binding sites (blue tracks) across the FEN1 gene clearly demonstrated the enrichment of R-loops and BRCA1 in the region amplified by the primers in FIG. 39A. FIG. 39C is a graphical representation of results from qPCR analysis similar to FIG. 39A with primers amplifying a region within the PARP8 gene.

As the data indicated that BRCA1 sequestration in EwS cells was R-loop dependent (FIG. 36A), a well-known R-loop region was further evaluated (FIGS. 39D and 39E) and BRCA1 binding was detected. FIG. 39D is a graphical representation of results from rqPCR analysis as in FIG. 39A with primers targeting a well-known R-loop region within APOE gene. FIG. 39E is a diagrammatic representation of gene expression (red tracks), R-loop sites (black and grey tracks) and BRCA1 binding sites (blue tracks) across the APOE gene again demonstrated the enrichment of R-loops and BRCA1 in the region amplified by the primers in FIG. 39D. Again, there was a significant damage-dependent decrease in BRCA1 binding in IMR90 and U2OS at this R-loop site, which was absent in the EwS cell lines. Finally, the presence of both RNAPII (by ChIP-qPCR) and R-loops at BRCA1 binding sites was confirmed by DRIP-PCR (FIGS. 39F and 39G). FIG. 39F is a graphical representation of results from qPCR analysis as in FIG. 39C but using RNAPII ChIP samples indicating levels of RNAPII occupancy among the different cell lines with and without treatment. FIG. 39G is a photograph of agarose gel blots evaluating amplicons generated using EWS502 DRIPs with primers against FEN1 and PARP8 genes. The data demonstrated that DRIP sites exist across regions that have BRCA1 binding. NT: No treatment. Etop: etoposide treated (6 hours) and RNH: RNaseH treated samples. **P<0.005, bar graphs represent means with SE, n=3.

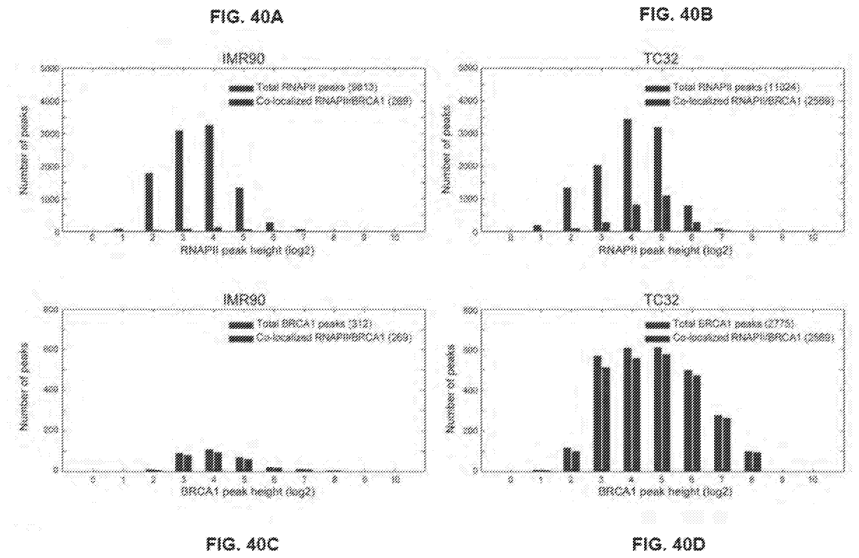

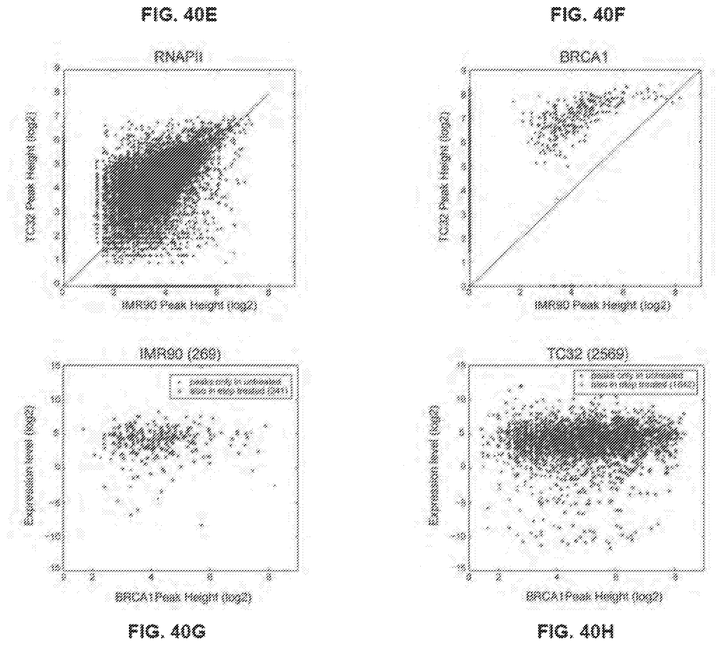

Genome-wide maps of BRCA1 sites centered around the transcription start sites indicated that highly active gene loci (gene expression and RNAPII localization) were associated with both BRCA1 and RNAPII localization (FIG. 37F). The ChIP-Seq data was analyzed to examine the relationship between BRCA1- and RNAPII-bound transcription start sites, and many more sites in the genome were bound by both in EwS (2569 genes) compared to IMR90 (269 genes) (FIGS. 40A-40H) corroborating the co-IP data that there is an enhanced interaction between RNAPII and BRCA1 in EwS.

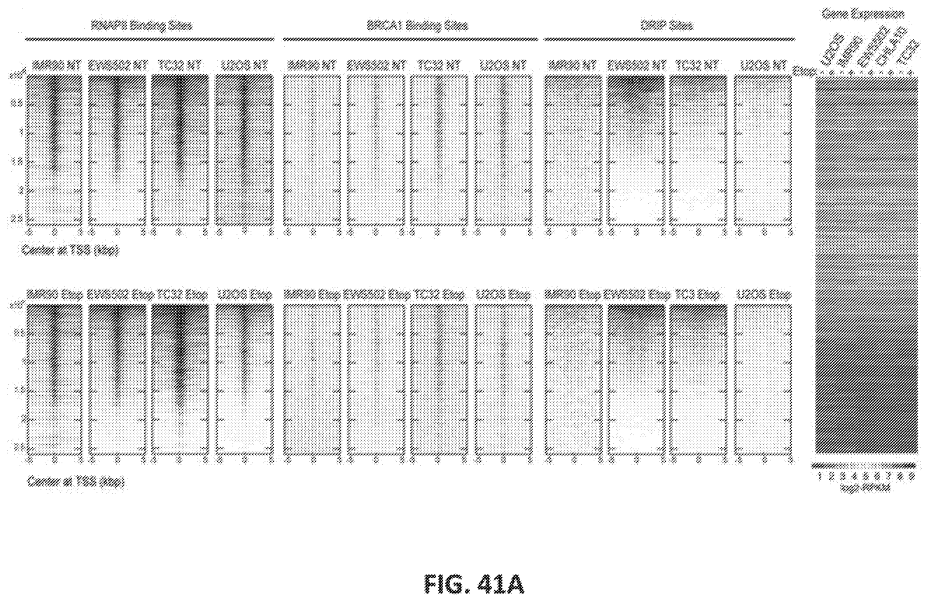

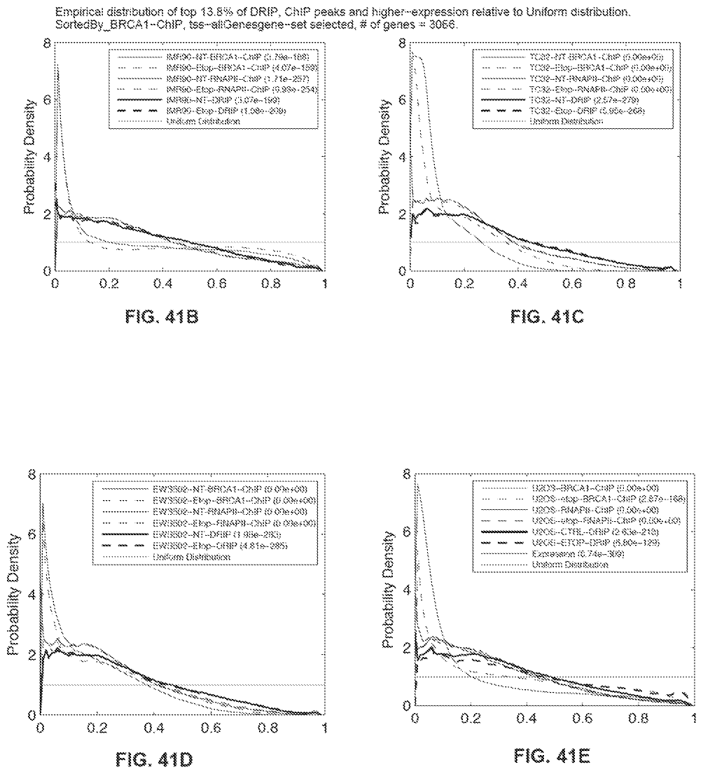

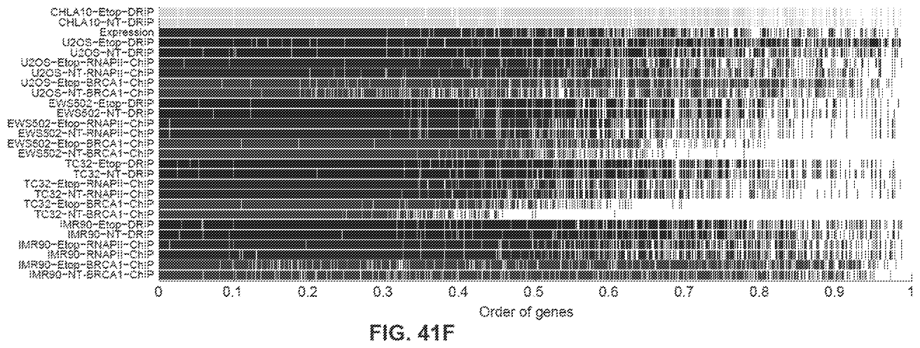

FIGS. 40A and 40B are graphical representations of the distribution of RNAPII abundance bound to DNA across the genome for IMR90 and TC32, respectively. The bars depict number of RNAPII bound sites as a function of number of peaks (Y-axis) and the relative abundance of RNAPII (peak height, log-transformed) within these peaks (X-axis). The blue bars indicate total number of peaks determined from RNAPII ChIP-seq and the red bars represent the peaks that co-localize with BRCA1 peaks obtained from BRCA1 ChIP-seq. The data indicated a similar number of RNAPII peaks for TC32 (11024) compared to IMR90 (9813), but a greater amount of DNA bound at these peaks implying increased RNAPII binding. Further, a higher proportion of RNAPII bound loci also co-localized to BRCA1 binding sites (red bars) in TC32 compared to IMR90 (23% compared to 2.7%) and there was a clear increase in RNAPII abundance at these sites. FIGS. 40C and 40D are graphical representations of the distribution of BRCA1 abundance across the genome for IMR90 and TC32, respectively. Similar to FIGS. 40A and 40B, the bars depict amount of BRCA1 binding sites as a function of number of peaks (Y-axis) and the relative abundance of BRCA1 (peak height, log-transformed) at these peaks (X-axis). The blue bars indicate total number of peaks determined from BRCA1 ChIP-seq and the red bars represent the peaks that co-localize with RNAPII peaks obtained from RNAPII ChIP-seq. The data indicated a significantly higher number of total BRCA1 peaks in TC32 as well as a significantly higher enrichment of BRCA1 within these peaks in TC32 compared to IMR90. The data also suggested that a majority of the BRCA1 peaks were co-localized with RNAPII. FIGS. 40E and 40F are scatter plots representing the correlation of RNAPII (FIG. 40E) and BRCA1 (FIG. 40F) peak heights between TC32 and IMR90. Data was plotted after it was normalized to read count and log-transformed to make comparisons. Loci that are unique to each cell line map to the axes while common loci are scattered around the diagonal. The data clearly suggested an increase in enrichment of both RNAPII and BRCA1 in TC32 compared to IMR90. FIGS. 40G and 40H are scatter plots representing the relationship between co-localized BRCA1 and RNAPII peaks as a function of BRCA1 peak height (X-axis) and level of expression of the gene associated with these binding sites (Y-axis) in IMR90 and TC32 cells, respectively. TC32 had a greater than 5-fold increase in the number of BRCA1 peaks that are associated with RNAPII at highly expressed genes. Further, like in FIG. 40B, there was a greater abundance of BRCA1 (peak height) at these highly expressed genes in TC32 compared to IMR90. There was a strong enrichment for BRCA1 and RNAPII binding at R-loops, particularly in the EwS cells (FIG. 37F and FIG. 41).

An in-depth analysis of the association of BRCA1 with R-loops was performed in control and Ewing sarcoma cell lines with and without exposure to damage (FIG. 41A). BRCA1 binding decreased significantly upon damage in control cells, but not as much in Ewing sarcoma cells. FIG. 41A is a heatmap representing genome-wide localization of RNAPII Binding, BRCA1 binding and R-loops centered around the transcription start site (TSS). The data was sorted by DRIP sites. The upper panel represents untreated (NT) samples and the lower panel represents cell lines exposed to etoposide (Etop) for 6 hours. Conversely, the presence of R-loops at BRCA1 binding sites was also confirmed. Genome-wide maps of BRCA1 sites centred on transcription start sites (TSS) indicated that highly expressed gene loci were associated with both BRCA1 and RNAPII localization (FIGS. 41B-41E). FIGS. 41B-41E are KS plots to demonstrate enrichment of BRCA1, RNAPII and R-loops at highly expressed genes. These KS plots demonstrate empirical distribution of the top 13.8% of DRIP and ChIP peaks and higher expression relative to uniform distribution. FIG. 41F is an illustration of the data as sorted by BRCA1 ChIP, n=3,066 genes. There was strong enrichment for BRCA1 and RNAPII binding at R-loops, particularly in the Ewing sarcoma cells (FIG. 41F). Increased co-localization was found between BRCA1- and RNAPII-bound transcription start sites in Ewing sarcoma (2,569 genes) compared to IMR90 cells (269 genes) (FIGS. 40A-40H), corroborating the co-immunoprecipitation data. An increase in enrichment (peak height) of these two proteins was found in TC32 compared to IMR90 cells. Collectively, the above results indicate that in Ewing sarcoma, BRCA1 is retained at stalled transcription complexes associated with R-loops, presumably to mitigate associated damage.

In comparison to IMR90, U2OS was not statistically significant in terms of enrichment, while the EwS cell lines were (Table 8). The data was generated by comparing RNAPII ChIP and R-loop probability distributions for all cell lines against IMR90 DRIP data centered around the transcription start site. The top 27% of DRIP-seq peaks corresponding to 6127 genes were used for the analysis and data was sorted by BRCA1 binding sites. The results clearly demonstrated enrichment of EwS R-loops and RNAPII binding over BRCA1 compared to IMR90 or U2OS.

TABLE 8
KS test for estimating enrichment of RNAPII
and R-loop binding at BRCA1 sites.

| | P-values of enrichment |
|---|---|
| Phenotype | over IMR90-DRIP |
| IMR90 DRIP | 1.00E+00 |
| IMR90 RNAPII | 2.59E−12 |
| U2OS DRIP | 0.9959 |
| U2OS RNAPII | 1.90E−06 |
| TC32 DRIP | 4.94E−08 |
| TC32 RNAPII | 4.87E−65 |
| EWS502 DRIP | 1.48E−10 |
| EWS502 RNAPII | 3.60E−44 |

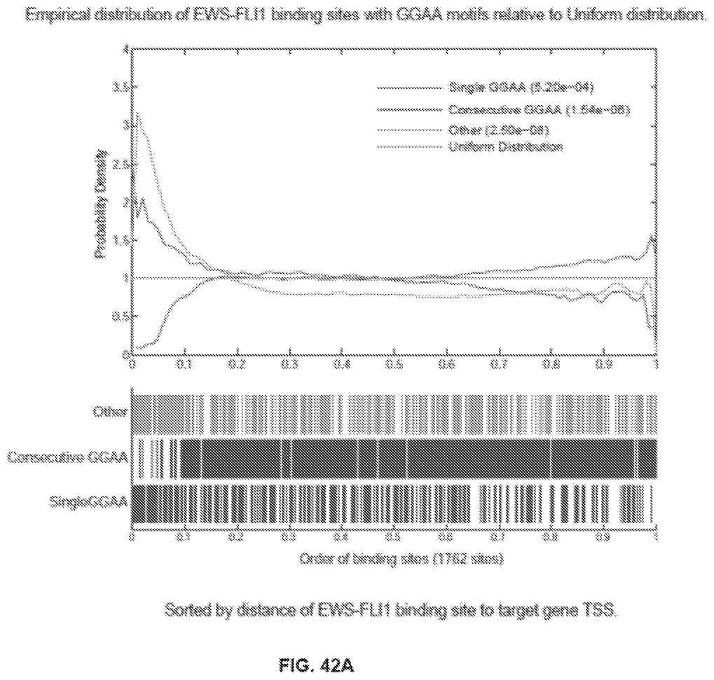

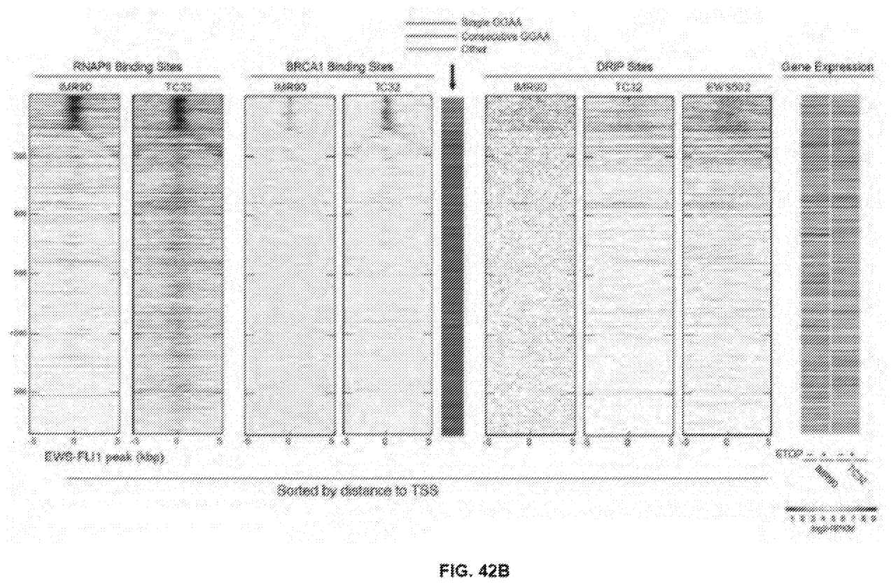

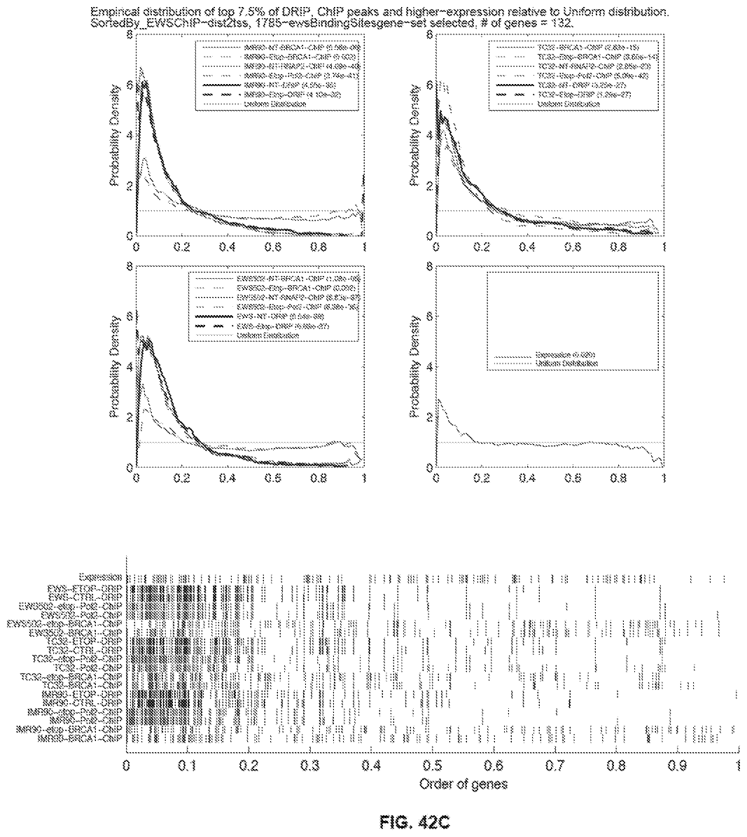

As EWS-FLI1 is a transcription factor, the genes associated with EWS-FLI1 binding sites were examined, observing that the greatest impact on gene expression, BRCA1 binding and R-loop levels was for binding sites more proximal to gene TSS (FIGS. 42A-42C). FIG. 42A is a probability density plot of the distribution of EWS-FLI1 binding peaks as a function of underlying sequence characteristic across all the 1762 unique genes (associated with the EWS-FLI1 binding sites) relative to uniform distribution. The data was ordered according to the distance of the binding peak to transcription start site (TSS). The KS plot indicated that the majority of canonical ETS transcription factor binding sites (GGAA; Single GGAA; blue) are located proximal to the TSS while the Consecutive GGAA (multiple consecutive copies of GGAA; red) are more distal. EWS-FLI1 binding sites not identified as being Single GGAA or Consecutive GGAA were classified as “Other” (green). Number of genes used in analysis=1762. P-values depicting significance of enrichment for each sample is indicated in the inset boxes. FIG. 42B presents heatmaps of gene expression, RNAPII, BRCA1 and R-loop sites at EWS-Fill bound genes (1762) centered on EWS-FLI1 peaks. Data was ordered by the distance to target gene TSS. The blue/red/green bars in the middle indicate the distribution of type of underlying sequence of the EWS-FLI1 binding sites. There was a strong enrichment of BRCA1 and DRIP sites in EwS at EWS-FLI1 sites that are around the TSS. FIG. 42C is a set of KS plots depicting enrichment of BRCA1, RNAPII and R-loops within the top 7.5% of EWS-FLI1 binding sites. Data was ordered by distance to TSS. P-values depicting significance of enrichment for each sample is indicated in the inset boxes.

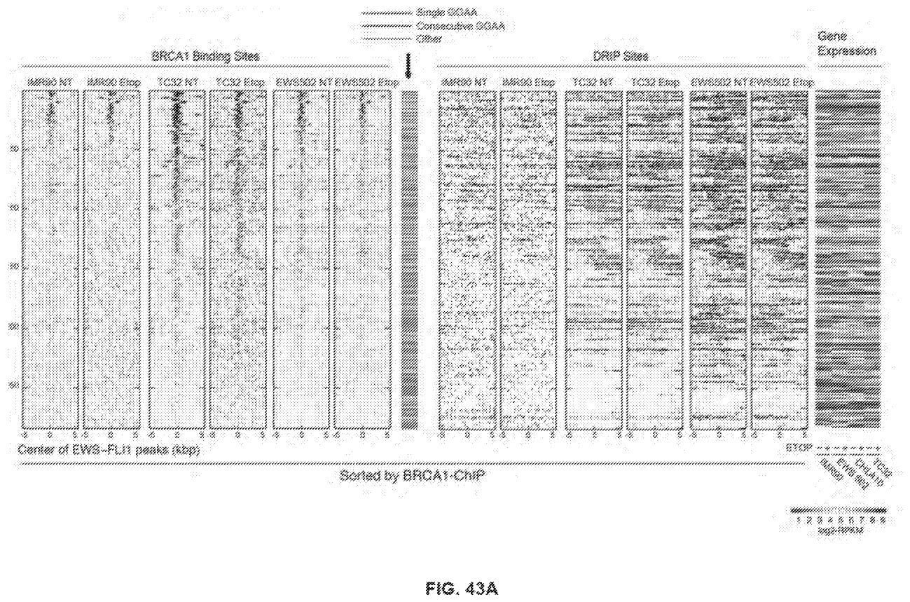

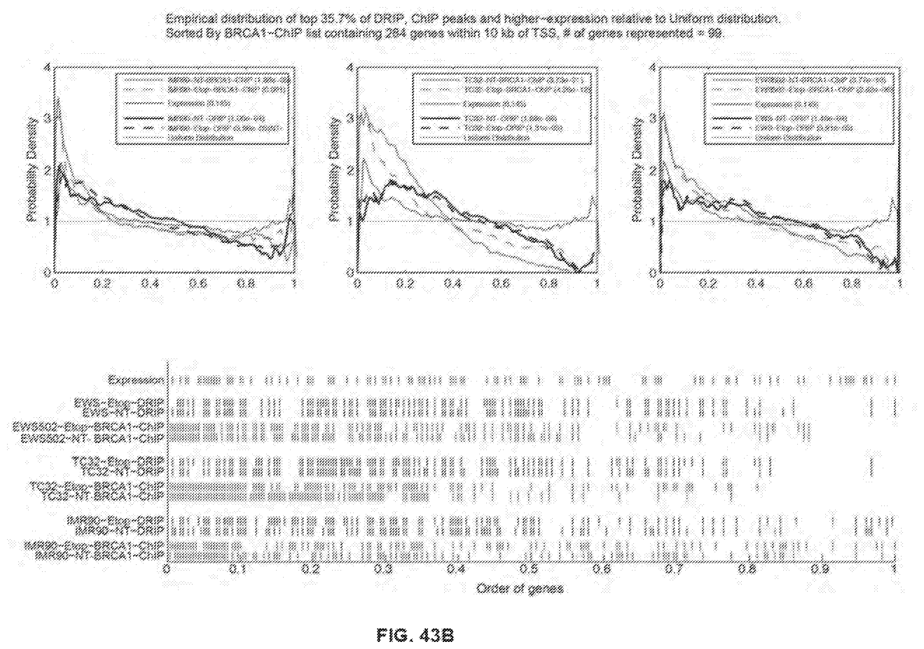

For those genes with EWS-FLI1 binding site within 10 kbp of a gene TSS, more BRCA1 binding was observed in EwS than in IMR90 cells and this binding did not reduce in response to etoposide as much in EwS compared to IMR90 (FIGS. 43A-43B). FIG. 43A is a heatmap of gene expression, BRCA1 and R-loop sites at genes associated with EWS-FLI1 binding proximal (<10 kb) to its TSS (n=284 sites or 280 unique genes; Table 12). Data was ordered by BRCA1 ChIP. The blue/red/green bars in the middle indicate the distribution of type of underlying sequence of the EWS-FLI1 ChIP. EwS cells demonstrated BRCA1 binding to a significantly higher proportion of genes than IMR90. Following damage (Etop), BRCA1 localization at IMR90 promoters was diminished, whereas this decrease was less apparent in EwS. FIG. 43B is a set of KS plots depicting enrichment of BRCA1 and R-loops within the top 35% of promoter-proximal EWS-FLI1 binding sites. Data was ordered by BRCA1 ChIP. P-values depicting significance of enrichment for each sample is indicated in the inset boxes.

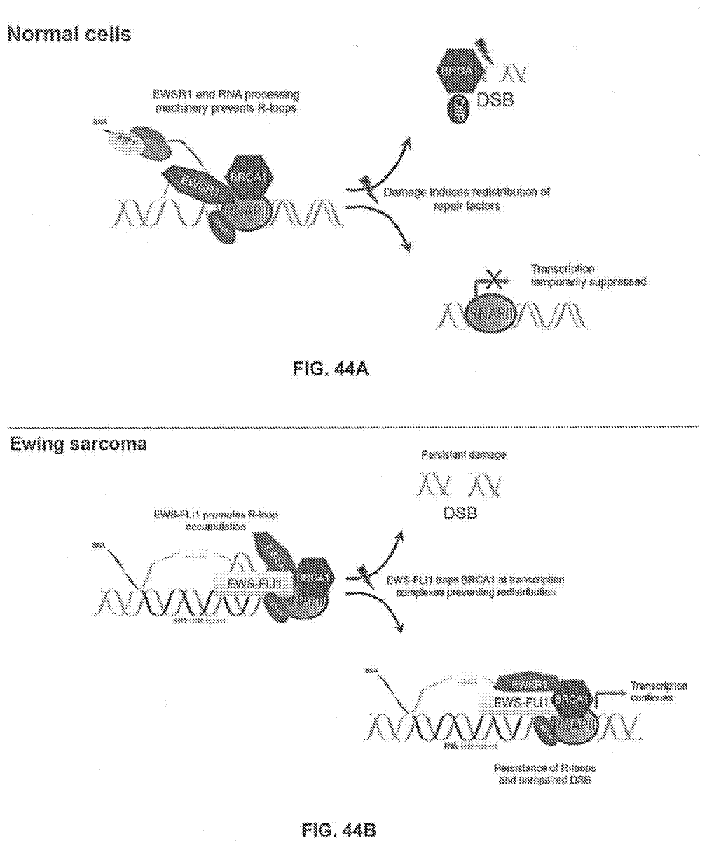

Collectively, the above results indicate that BRCA1 is retained at stalled transcription complexes associated with R-loops (FIGS. 44A-44B), presumably to mitigate associated damage. FIG. 44A is a diagrammatic representation of the BRCA1 interactions in normal cells. Normally, BRCA1 interacts with the hyperphosphorylated RNAPII. Upon genotoxic stress, BRCA1 is released from this complex and associates with several repair complexes and proteins (including CtIP) to ensure faithful repair of damaged DNA. FIG. 44B is a diagrammatic representation of the BRCA1 interactions in Ewing sarcoma cells. The BRCA1/phosphorylated RNAPII interaction is highly augmented due to EWS-FLI1 dependent aberrant transcription. BRCA1 is unable to dissociate from the transcription complex upon damage thereby rendering EwS cells HR deficient.

In an embodiment of a method for diagnosing and treating cancer in a subject, the tissue sample from the subject is analyzed for phosphorylation levels of RNA polymerase II. If an increase in phosphorylation levels of RNA polymerase II is detected, the subject is diagnosed with the cancer. The method further includes the step of administering such a subject with a therapeutically effective amount of an inhibitor of ATR and an inhibitor of PARP1 or pharmaceutical acceptable salts thereof to the diagnosed subject. The method further includes the step of administering such a subject with a therapeutically effective amount of an inhibitor of CHK1 and an inhibitor of PARP1 or pharmaceutical acceptable salts thereof to the diagnosed subject. Disclosed here are methods of stratifying patients by immunohistochemical assays to assess relative levels of R-loops and phosphorylated RNA polymerase II.

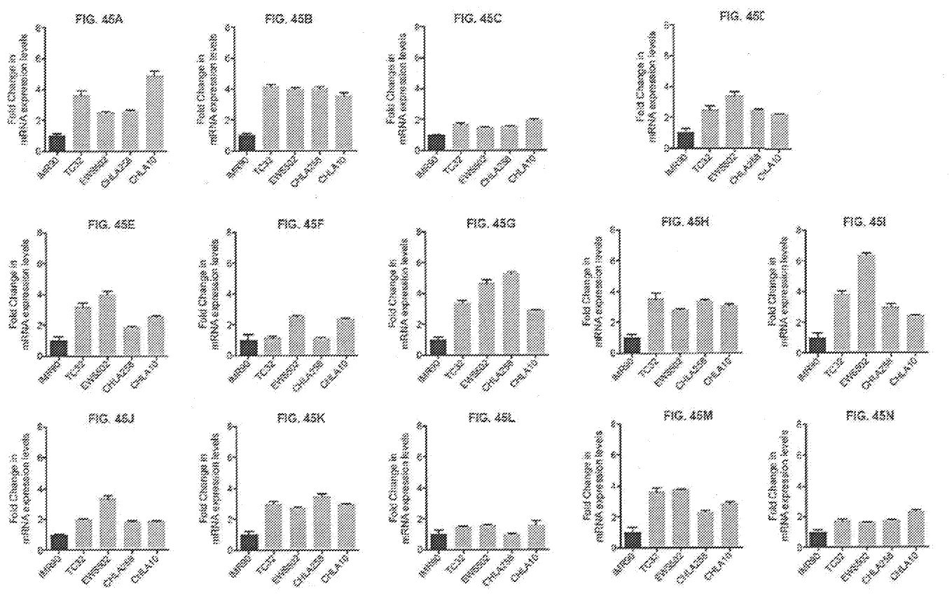

EwS tumors reveal upregulated processes that counteract “genomic stress” EWS-FLI1 expression is known to be toxic to most cells in which it is introduced. The high levels of R-loops, replication stress and repair defects observed with EWS-FLI1 expression provide an explanation for this, but suggest the presence of compensatory mechanisms present in EwS and possibly in its highly debated cells-of-origin. The RNA-seq data was mined and increased expression of several pathways was identified that could counteract either the presence of R-loops or the resultant replication stress. Most interestingly, all EwS cell lines had an over two-fold increase in the expression of RNASEH2 complex subunits compared to IMR90 (FIG. 45A-45C). FIG. 45A is a graphical representation of the upregulation at transcript levels of genes in the RNASEH2A complex. FIG. 45B is a graphical representation of the upregulation at transcript levels of genes in the RNASEH2B complex. FIG. 45C is a graphical representation of the upregulation at transcript levels of genes in the RNASEH2C complex. FIG. 45D is a graphical representation of the upregulation at transcript levels of FEN1. FIGS. 45E-45N are graphical representations of the upregulation at transcript levels of genes in the Fanconi Anemia pathway: FANCA (FIG. 45E); FANCB (FIG. 45F); FANCC (FIG. 45G); BRCA2 (FIG. 45H); FANCD2 (FIG. 45I); FANCE (FIG. 45J); FANCG (FIG. 45K); FANCL (FIG. 45L); FANCI (FIG. 45M); and FANCM (FIG. 45N).

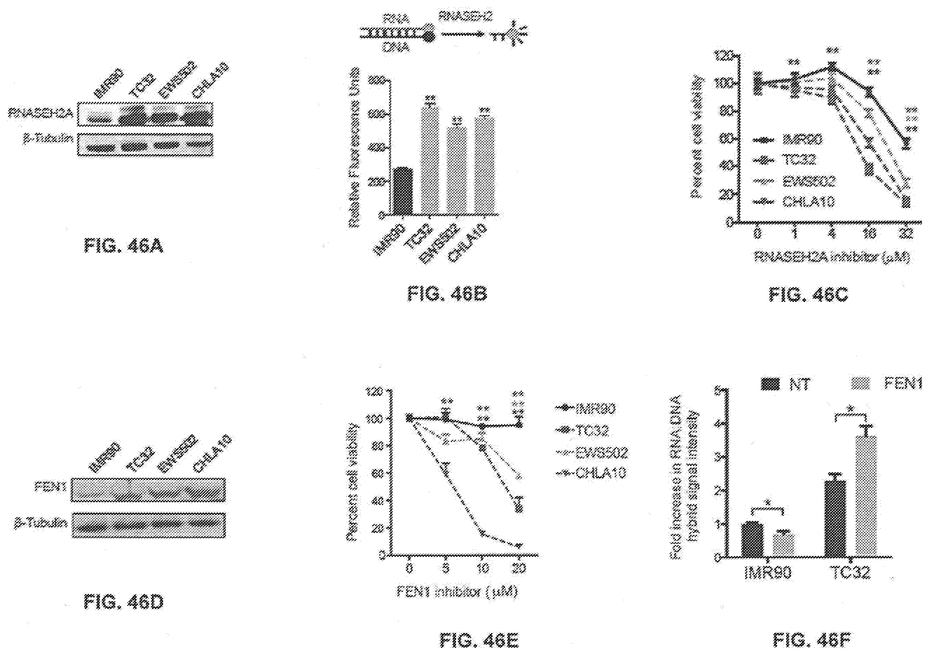

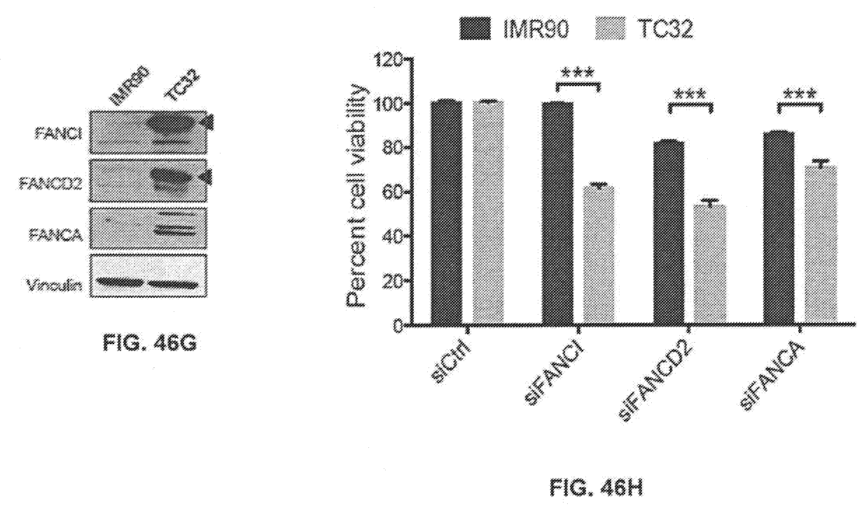

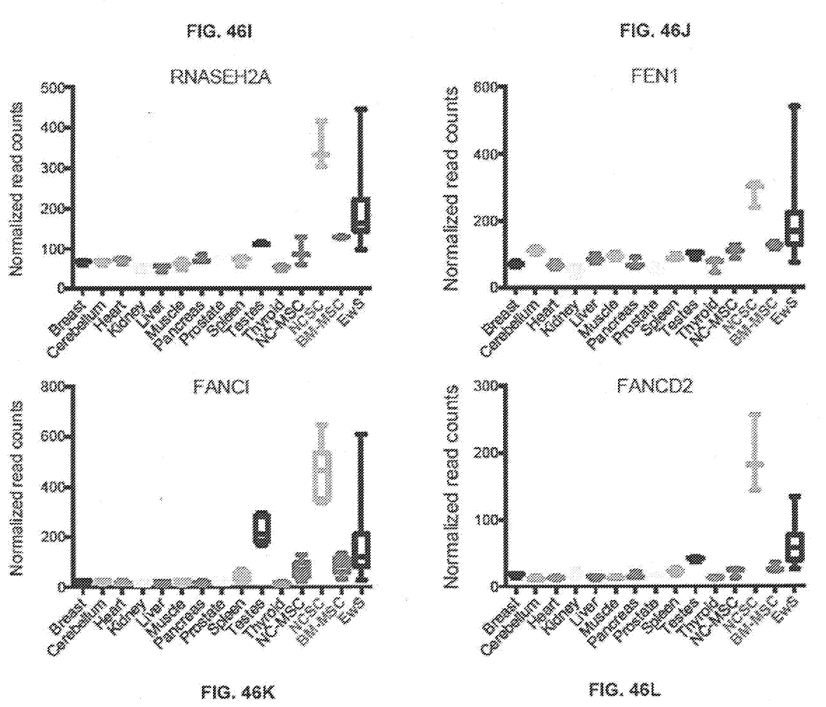

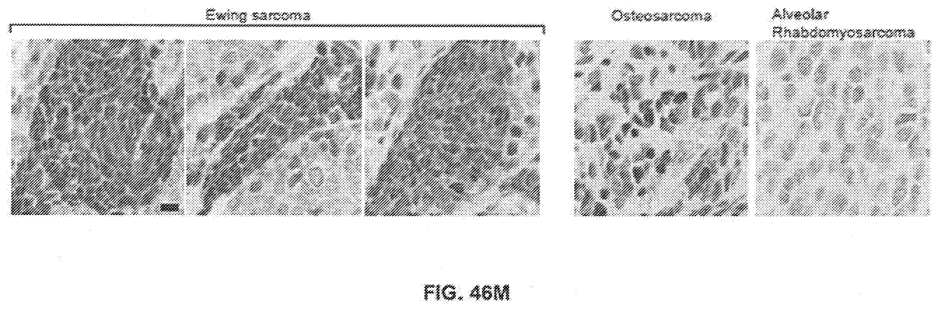

We confirmed not only an increase in RNASEH2A protein levels (FIG. 46A), but also increased activity (FIG. 46B). FIG. 46A is a photograph of an immunoblot showing increased basal expression of RNASEH2A in EwS cell lines. FIG. 46B is a graphical representation of the levels of RNASEH2A activity in hydrolyzing RNA in RNA:DNA hybrids measured using a fluorometric assay with whole cell lysates and substrate as indicated in the schematic. Signals were normalized relative to IMR90 activity. FIG. 46C is a graphical representation of cell viability curves in response to increasing doses of RNASEH2 inhibitor R11 measured 72 hours after addition of the drug. EwS cell lines also demonstrated significantly higher sensitivity to an RNASEH2A inhibitor compared to IMR90 (FIG. 46C). Flap structure-specific endonuclease 1 (FEN1) is an essential enzyme that removes 5′ overhangs during DNA repair and replication. Recently, FEN1 was implicated in processing RNA:DNA hybrids at telomeres to limit telomere fragility. FEN1 was upregulated in EwS cell lines (FIG. 46D and FIG. 45D), and its chemical inhibition resulted in severe toxicity in EwS (FIG. 46E). FIG. 46D is a photograph of an immunoblot showing increased levels of FEN1 in EwS cells. FIG. 46E is a graphical representation of cell viability measured 72 hours after incubation with an inhibitor of FEN1 endonuclease activity caused significant cell death in EwS. R-loops were evaluated as a potential substrate for FEN1. This seemed to be the case in EwS, as unlike in IMR90, FEN1 inhibition (FIG. 46F) led to further accumulation of R-loops in TC32. FIG. 46F is a graphical representation of R-loop signal intensity in genomic DNA of cells that were treated with 10 μM FEN1 inhibitor (FEN1i) for 6 hours, as measured by dot blot assay and quantified using ImageJ software. Fold change was calculated relative to IMR90 untreated. NT: No treatment.

Another pathway upregulated across EwS cell lines was the Fanconi Anemia (FA) pathway (FIGS. 45E-45N). FA genes play a critical role in resolution of crosslink damage, R-loop resolution and maintaining replication fork integrity to preserve genome stability. Mono-ubiquitination of FANCD2 and FANCI are critical steps in activating the core ubiquitinating complex to stabilize the stalled fork. FIG. 46G is a photograph of an immunoblot showing levels and activation of proteins involved in FA pathway in whole cell lysates of control and EwS (TC32); the mono-ubiquitination of FANCI and FANCD2 are indicated by black triangles. FIG. 46H is a graphical representation of the knockdown of several FA genes induced significantly more cell death in TC32 compared to IMR90 measured 72 hours after knockdown. FIGS. 46I-46L are box and whisker plots depicting gene expression levels of upregulated factors across normal tissues (triplicate) and 32 primary EwS tumor samples. FIG. 46M is a set of representative immunohistochemistry images showing R-loops (S9.6) staining across a sarcoma tissue microarray. Scale bar (thick black line in the first image) represents 10 μm. *P<0.05, **P<0.005, the colors of the * match that used for the cell line in the multi-cell line cytotoxicity plots and were tested against control. Line and bar graphs display means with SE, n=4.

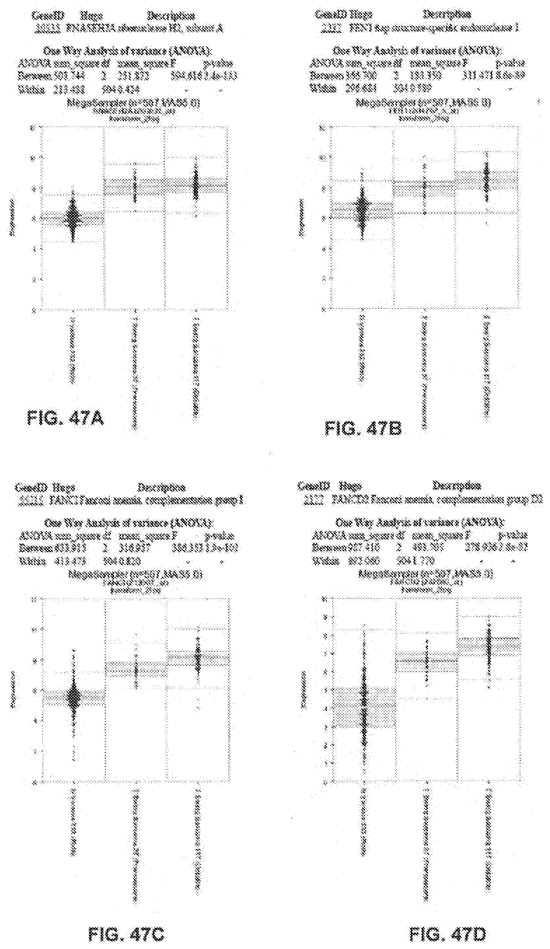

Dramatic hyperactivation of FANCD2 and FANCI was observed in EwS cells (black triangles, FIG. 46G) and a marked increase in cell death with knockdown of multiple FA genes (FIG. 46H) suggesting a dependence on this pathway for EwS survival. If these pathways are critical for EwS survival in the context of EWS-FLI1 induced oncogenic stress, the following issues were evaluated1) the upregulation of these pathways could be detected in primary tumors, and 2) these pathways were similarly upregulated in putative cells-of-origin tissues (either neural crest stem cells (NCSC)68 or bone marrow derived mesenchymal stem cells (BM-MSC). A similar increased expression level was found for RNASEH2A, FEN1 and FA genes for 32 EwS tumors (pre-treatment) compared to 14 other tissues, with the exception of NCSCs and to a lesser extent BM-MSCs (FIGS. 46I-46L). The results clearly demonstrate that these two progenitor stem cell populations provide the appropriate cellular context to tolerate EWS-FLI1 expression. The R2 database containing gene expression data of 353 normal tissue and 154 EwS tumor samples from independent studies was mined and upregulation of the afore-mentioned pathways was confirmed (FIGS. 47A-47D) in EwS tumors.

FIG. 47A is a graphical representation of gene expression of RNASEH2A across independently generated datasets. FIG. 47B is a graphical representation of gene expression of FEN1 across independently generated datasets. FIG. 47C is a graphical representation of gene expression of FANCI across independently generated datasets. FIG. 47D is a graphical representation of gene expression of FANCD2 across independently generated datasets. Data analysis was done using the R2 database portal. Roth: A panel of 353 normal tissues, Francesconi: 37 Ewing sarcoma tumors, Delattre: 117 Ewing sarcoma tumors.

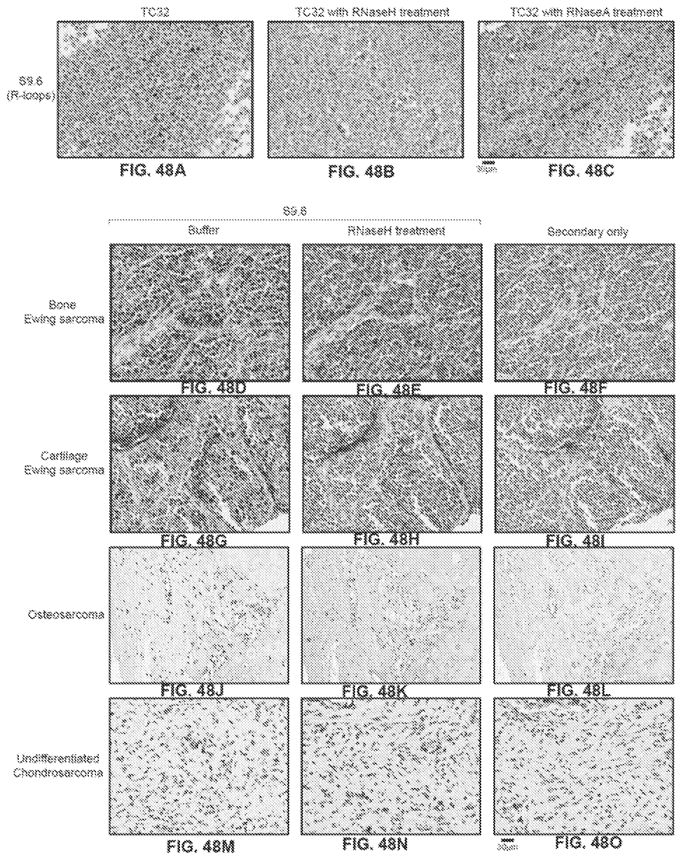

Finally, immunohistochemical analysis was performed on a sarcoma tissue microarray stained with the S9.6 antibody (FIG. 46M and FIG. 48A-48O) and confirmed the higher accumulation of R-loops in EwS primary tumors compared to other types of sarcomas. Taken together, the above results indicate that EwS counteracts the high levels of genomic stress (replication, recombination and transcription defects) by upregulating R-loop/replication stress resolution pathways. Most importantly, the similarity in upregulated pathways between our cell lines and the primary tumor samples provide clinical relevance to our study.

FIGS. 48A-48C are photographs of immunohistochemical images depicting R-loop staining by S9.6 antibody on sections derived from fixed TC32 cell pellets. Sections were either treated with buffer (FIG. 48A), RNaseH (FIG. 48B) or RNaseA (FIG. 48C) after antigen retrieval. The slides demonstrated loss of R-loop signal after treatment with RNaseH as expected. RNaseA treatment which at higher salt concentrations specifically cleaves single-stranded RNA did not result in a significant loss of R-loop signal confirming the specificity of S9.6 antibody in detecting RNA:DNA hybrids. FIGS. 48D-48O are photographs of Representative IHC images from a pan-sarcoma tissue microarray. The left and center panels were probed with S9.6 antibody with or without RNaseH treatment. The right panel was stained with secondary antibody alone and serves as a non-specific control. Each row represents images from one tumor indicated on the left. Images were scanned at 40× and the bar at the bottom right denotes the resolution.

The exacerbated transcription and replication stress caused by EWS-FLI1 is a contributing factor to the exquisite chemosensitivity of these tumors. The EWS-FLI1 fusion protein is known to drive an oncogenic transcriptome, but here it ha been demonstrated that it also interferes with EWSR1 function, thereby contributing to EwS pathogenesis. EWSR1 inhibits the activation of RNAPII whereas EWS-FLI1 promotes the same, even in the presence of wild type EWSR1. This data provides evidence of a potential dominant-negative function of EWS-FLI1 over EWSR1.

EWSR1 inhibits R-loop accumulation and facilitates HR. EWSR1 may be important for the release of BRCA1 from those transcribed genes with which it associates, and the concerted involvement of both proteins is essential for efficient recombination upon damage. The widespread occurrence of R-loops in conjunction with impaired HR led us to examine BRCA1 effect on R-loop accumulation; neither knockdown nor overexpression of BRCA1 significantly altered R-loop levels in EwS. This suggests that BRCA1 participates in reducing the downstream consequences of R-loop formation. BRCA1 retention at transcription complexes in EwS precludes its participation in HR, thereby explaining the acute sensitivity to PARP1 inhibitors. Therefore, this study also reveals a new class of BRCA1-mutant-like tumors that could benefit from PARP1 inhibitor treatments. However, chemoresistance mechanisms prevalent in breast carcinomas would also likely confer PARP1 inhibitor resistance in EwS, which may explain the lack of response observed in single agent PARP1 inhibitor clinical trials with relapsed patients. DNA damage is known to induce profound changes to the transcriptional landscape. Evidence of an active link between transcription stress and HR has been provided; EWSR1 dependent release of BRCA1 from transcription complexes following damage is necessary for efficient HR. However, the mechanism by which this occurs remains an open question. BRCA1 predominantly forms a heterodimer with BARD1 and both EWSR1 and EWS-FLI1 can interact with BARD1. Therefore, one possibility is that EWS-FLI1's association with BARD1 at transcription complexes indirectly prevents BRCA1 release upon damage. Alternatively, BRCA1 association with RNAPII could be a licensing mechanism to regulate damage-associated transcriptional events, which may be abrogated in EwS. C ells undergo a transient decrease in transcription post-damage, and considering the dependency upon transcription, there is also a decrease in R-loops, which is largely absent in EwS. Therefore, EWS-FLI1 may mediate BRCA1 retention at a subset of genes to alter their normal response to damage.

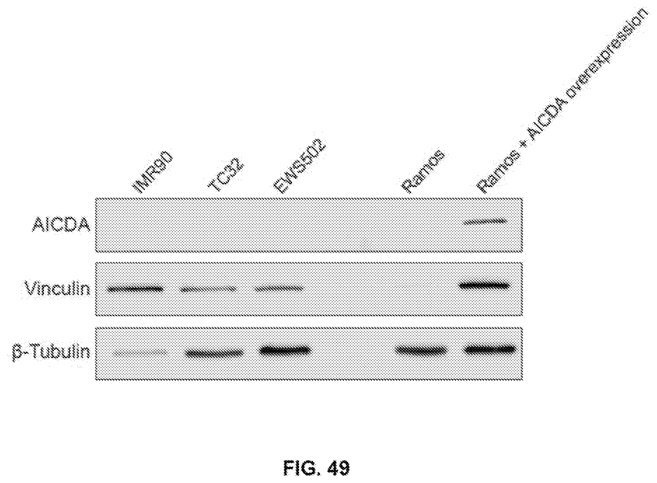

R-loops were thought to promote mutagenesis, but genomic instability is largely absent in EwS except post treatment. One explanation could be the upregulation of various DNA repair pathways in EwS, which was observed via gene expression analysis. EwS (both cell lines and primary tumors) has negligible expression of the enzymes involved in inducing R-loop-dependent mutations—the APOBEC genes (data not shown) and c-AID (FIG. 49). FIG. 49 is an immunoblot showing levels of c-AID (AICDA gene) enzyme across a panel of control and EwS whole cell lysates. Lysates from Ramos cells and Ramos cells overexpression AICDA were purchased from Novus Biologicals. Vinculin and β-Tubulin are provided as alternative loading controls. Finally, inhibitors targeting these EWS-FLI1 compensatory pathways can function either as single agents or in combination with chemotherapy to augment therapeutic window. The chemoresistant EwS cell line CHLA10 (obtained from a heavily pre-treated recurrent patient tumor) is highly sensitive to FEN1 inhibitor. A significant aspect of EwS tumorigenesis and chemosensitivity can be attributed to interference with wild type EWSR1 function. In fact, mutations in TET proteins are associated with several therapeutically challenging cancers. Agents that induce transcription or replication stress can be potentially effective treatment strategies in various tumors associated with EWSR1-translocations.

### Methods

Cell culture and transfections. Ewing sarcoma cell lines (TC32, CHLA10 and CHLA258) were obtained from Children's Oncology Group and EWS502 was obtained from Dr. Lessnick. Of note, TC32 is highly chemosensitive and CHLA10 was derived from a post-treatment recurrent chemoresistant tumor (Table 1). As controls, IMR90 (primary human neonatal fibroblast) and U2OS (pediatric Osteosarcoma) cell lines were obtained from ATCC. IMR90 and U2OS were cultured in DMEM (Corning), TC32, EWS502, RDES in RPMI-40 (Corning), SKES1 in McCoys (Corning) and CHLA10, CHLA258 in IMDM (HyClone); all cultured media were supplemented with 10% FBS (Atlanta Biologicals) and 1% antibiotic/antimycotic solution (Corning). Cells were maintained at 37° C. in a humidified atmosphere with 5% CO2 and tested for mycoplasma contamination.

All transfections were carried out using Lipofectamine RNAiMax, Lipofectamine 3000 (Invitrogen) following manufacturer's instructions. For RNASEH1 transfections, 3×10⁶ TC32 cells were transfected with 13 μg of plasmid DNA using Amaxa nucleofection (program X-001). Gene knockdowns were performed by reverse transfection whereas plasmid transfections were performed 24 hours after seeding. The siRNAs used in this study include: BRCA1 and EWSR1 (Life Technologies), EWSR1, BRCA1, FLI1, FUS, TAF15, 53BP1, FANCA, FANCD2 and FANCI (Santa Cruz). For some experiments, lentiviral transduction of TC32 with shCtrl (clone RHZ4743, Life Technologies) or shFLI1 (clone V2THS227524 or V3THS414176, Life Technologies) was performed. EWS-FLI1, EWS-FLI1 R2L2, EWS-ERG, EWSR1 (full length) and shEWSR1 in pMSCV vector were obtained from Dr. Stephen Lessnick. EWSR1 N terminal domain in pLX304 vector was purchased from DNASU. GFP-RNASEH1 and FLAG-RNASEH1 plasmids were a kind gift from Dr. Robert Crouch (Eunice Kennedy Shriver National Institute of Child Health and Human Development). HA-BRCA1 was obtained from Dr. Yuzuru Shiio (UTH-SA). ISceI in pCAGGS vector was a kind gift from Dr. Maria Jasin (Memorial Sloan Kettering Cancer Center) and Dr. Jeremy Stark (City of Hope Cancer Center). All siRNA and plasmid transfections were accompanied with control siRNA or empty vector respectively.

Cell viability. Cells were seeded at 30% confluence in 96 or 384 well plates with or without reverse transfection with siRNA. For plasmid expression, cells were transfected in 60 mm dishes first and then split into 96 or 384 well plates. The following day, the cells were treated to different drugs/inhibitors and cell viability was evaluated after 48 to 72 hours using Celltiter-Glo (Promega). Each condition was tested in triplicate. Chemicals used in cell viability experiments include etoposide, camptothecin, 5,6-dichloro-1-β-D-ribofuranosyl-1H-benzimidazole (DRB), methyl methane sulfonate (MMS), hydroxyurea, Mitomycin C, VE-821 (ATR inhibitor), FEN1 inhibitor (RF00974SC, Maybridge Chemicals), RNASEH2A inhibitor (R11, AG-690/40083036, Specs Labs), Olaparib (PARP1 inhibitor, Selleck Chemicals) and zeocin. Drug screening data from an independently published study was obtained from http://www.cancerrxgene.org/.

Immunoblotting and Immunoprecipitation. Whole cell lysates were prepared using RIPA buffer according to standard protocols. Subcellular protein fractionation kit for cultured cells (ThermoFisher Scientific) was used to extract nuclear and chromatin fractions. Cell lysates were separated on 3-8% or 4-12% gradient gels (Invitrogen) and transferred onto nitrocellulose membrane. All blots were incubated with primary antibody overnight and developed using enhanced chemiluminescence (Super ECL, ThermoFisher). Antibodies used in this study include FLI1 (ab15289, Abcam), EWSR1 (ab133288 and ab54708, Abcam), RNAPII phospho Ser2 (MMS-129R-200, Covance), RNAPII phospho Ser5 (61085, Active Motif), RNAPII (ab817, Abcam), RNA:DNA hybrids (S9.6, ENH001, Kerafast), single stranded DNA (ssDNA, MAB3034, Millipore), RNASEH1 (15606-1-AP, Proteintech), Nucleolin (sc-13057, Santa Cruz), 53BP1 (A300-272A, Bethyl labs), RAD51 (70-005-EX, Cosmo), FUS (cs4885, Cell Signaling), TAF15 (sc-81121, Santa Cruz), ATR phospho Ser428 (cs28539, Cell Signaling), ATR (sc-1887, Santa Cruz), CHK1 phospho Ser317 (cs2344, Cell Signaling), CHK1 (cs2345, Cell Signaling), RPA2 phospho Ser4/8 (A300-245A, Bethyl labs), RPA2 (ab2175, Abcam), BRCA1 (sc642, Santa Cruz and 07-434, Millipore), CtIP (cs9201, Cell Signaling), BARD1 (ab50984, Abcam), RNASEH2A (sc-101112, Santa Cruz), FEN1 (sc-13051, Santa Cruz), FANCA (sc-28215, Santa Cruz), FANCD2 (NB100-182, Novus), FANCI (A301-254A, Bethyl labs), FLAG-tag (ab1162, Abcam), Tata Binding protein (TBP, ab818), β-Actin (ab16039, Abcam), β-Tubulin (cs2128, Cell Signaling) and Vinculin (cs13901, Cell Signaling).

All co-immunoprecipitation experiments were done with endogenous proteins. Briefly, cells from nearly confluent 15 cm plate that was either treated with vehicle or LD35 dose of etoposide for 2 hours were harvested and initially lysed in cytosolic extraction buffer (low salt buffer—20 mM Hepes, pH 7.4, 0.5% Nonidet P-40, 10 mM NaCl supplemented with Halt protease and phosphatase inhibitors) for 30 min on ice. The nuclei were subsequently extracted in a high salt buffer (20 mM Hepes, 0.5% Nonident P-40, 1.5 mM MgCl₂, 0.5 M NaCl) for 45 min on a rocker at 4° C. The lysates were then diluted to physiological salt concentration (150 mM NaCl) and incubated for another 45 min. The nuclear lysates (0.5-0.75 mg) were pre-cleared using protein A/G beads and incubated overnight with 2 μg antibody on a rocker at 4° C. 25 μl of settled equilibrated protein A/G beads (or protein L for IgM antibody) were then added to the antibody:lysate mixture and incubated for further 6 hours. Bound complexes were washed three times in lysis buffer before elution with Invitrogen NuPage loading buffer. Eluted proteins were evaluated by immunoblotting. Co-IP experiments were repeated at least three times.

In vitro RNAPII phosphorylation assay. The following purified recombinant proteins were purchased: RNAPII CTD fragment (POLR2A-1149H, Creative BioMart), EWSR1 (TP303709, Origene), FUS (TP301808, Origene), CDK9/cyclin T1 (PV4131, ThermoFisher Scientific) and CDK7/cyclin H/MNAT1 (ab64303, Abcam). The in vitro kinase assay was performed using the Adapta kinase assay (PV5099, ThermoFisher Scientific) according to manufacturer's instructions. The template used for kinase activity was either 50 μM CDK7/9tide provided in the kit (PV5090, ThermoFisher Scientific), or 12 ng RNAPII CTD fragment. 1 μM EWSR1 or FUS was added to the substrate and 10 μM ATP kinase buffer and pre-incubated for 15 min. 1.77 μg/ml CDK9/cyclin T1 or 5.6 μg/ml CDK7/cyclin H/MNAT1 were then added and the kinase reaction was allowed to proceed for a further 45 minutes. 10 μM ADP and 10 μM ATP served positive and negative controls. Finally, Eu-labeled antibody and Alexa Fluor-647-labeled tracer added and the level of ATP consumption was measured 30 minutes later using BMG Labtech Pherastar microplate reader. Each condition was tested in quadruplicate and the overall experiment was repeated at least 3 times for independent validation.

Transcription Recovery Assay. Cells grown on fibronectin-coated coverslips were treated with LD50 doses of etoposide for 0, 2 and 16 hours, followed by incubation in medium containing 0.5 mM 5-Ethynyl Uridine (EU, ThermoFisher Scientific) for 3 0 minutes. Post-incubation, cells were fixed, permeabilized and subjected to Click-iT RNA reaction (Click-iT RNA Alexa Flour 598 Imaging kit, ThermoFisher Scientific) according to manufacturer's instructions. Washed coverslips were mounted onto glass slides using ProLong Antifade Mounting Solution with DAPI (ThermoFisher Scientific) and imaged using a Zeiss microscope at 63×. A minimum of 100 cells were analyzed for each condition. Image analysis was performed using ImageJ.

Dot Blot for R-loops. 0.5 μg of restriction enzyme digested genomic DNA was loaded on to pre-wet H⁺ nylon membrane. The membrane was washed twice with dH₂O, rinsed in 2×SSC buffer and then left to air-dry at room temperature. For ssDNA, an additional denaturation step (incubation in 0.5N NaOH, 1.5M HCl for 10 minutes), followed by a 10 minute incubation in neutralization buffer (1M NaCl, 0.5M Tris-HCl pH 7) was performed. The membrane was then blocked with 1× TBS containing 5% non-fat dry milk and incubated with the primary antibody (R-loops S9.6 antibody and ssDNA) in blocking buffer overnight. The blots were analyzed using ImageJ software or LI-COR Image Studio™ to measure signal intensity from genomic DNA, RNaseH treated DNA and ssDNA. ssDNA signal was used to normalize R-loop signal. Dot blot experiments were done in quadruplicate and repeated for validation.

Immunofluorescence. Cells were seeded on fibronectin-coated coverslips. Following knockdown and/or etoposide treatment for 6 hours, cells were fixed with 4% paraformaldehyde. For R-loops, a pre-extraction step was carried out by treating the cells with 0.1% triton-X 100 in PBS for 30 seconds at room temperature. After permeabilization with triton-X 100 for 10 min, cells were blocked for 1 hour with 1% BSA, 4% goat serum followed by overnight incubation with primary antibodies in blocking buffer. Coverslips were then incubated with Alex Fluor 488/568 conjugated secondary antibodies (Life Technologies). The cells were then stained with DAPI and coverslips were mounted on slides using Vectashield (Vector labs, Burlingame, Calif.). Cells were imaged using a Zeiss microscope at 40× or 63×. At least 100 nuclei per data set were sampled and a minimum of 80 nuclei per condition was used for quantification of immunofluorescence intensity or foci counting. Image analysis was done using Adobe Photoshop software.

DNA fiber analysis. Cells were pulse-labeled with IdU, then CldU for 30 min each. DNA fiber spreads were prepared as described previously. Cells were harvested, and lysed (50 mM EDTA, 0.5% SDS, 200 mM Tris-HCl pH 7.5) directly on a glass slide. DNA fibers were stretched out on a glass surface by placing it at an angle and then fixed with methanol:acetic acid (3:1) for 10 min and denatured with 2.5 M HC1 for 80 min. Nascently incorporated nucleotides were detected using primaries Anti-BrdU (BD Biosciences) and Anti-BrdU (AbD Serotec) followed by Alexa fluor 488 and 568 conjugated secondaries, respectively. Images were acquired using a 40× oil immersion objective and analyzed using Adobe Photoshop. A minimum of 80 fibers was examined for each experimental group performed in triplicate.

Immunohistochemistry. The sarcoma tissue microarray (T264) was purchased from US Biomax Inc. Tissue microarrays were treated with 1 mM EDTA pH 8 for 40 minutes at 95° C. followed by a 20 min cool down step. To confirm antibody specificity, slides were incubated with RNaseH (Cat #M0297, New England Biolabs) and RNaseA (Cat #EN0531, Thermo Fisher Scientific) enzymes 1 h and 24 h at 37° C. respectively as previously described by Chédin's Lab. Slides were then rinsed in 1× Tris Buffered Saline (TBS) three times. Following endogenous peroxidase blocking, the slides were incubated with S9.6 (1:20,000) for 2 hours at room temperature in a moist humidity chamber. Anti-mouse Powervision-HRP Conjugated Polymer from Leica (Cat #PV6114) for 30 minutes was used for detection. Slides were then developed with DAB for 5 minutes, rinsed with TBS and counterstained with hematoxylin, dehydrated, cleared and mounted with a synthetic mounting medium. Images were taken on the Olympus sc-100 at 60× magnification or the Motic Digital Slide Scanning System at 40× magnification.

Repair Assays. The DR-GFP reporter assay was carried out as previously described. U2OS cells with stably integrated DR-GFP construct and the endonuclease ISceI expression vector were obtained from Dr. Maria Jasin (Memorial Sloan Kettering Cancer Center) and Dr. Jeremy Stark (City of Hope Cancer Center). Briefly, cells were seeded in 24-well plate and transfected with siRNAs or expression vectors or combinations thereof. The following day, cells were transfected with IScel expression vector. After 72 hours, cells were harvested and GFP positive cells were evaluated by flow cytometry on a BD flow cytometer. Appropriate controls were used and all experiments were performed in triplicate.

BRCA1 ChIP-qPCR at DR-GFP sites. The protocol described in was followed for this assay. Briefly, DR-GFP U2OS cells were transfected with EWS-FLI1 or the empty vector control. Twenty-four hours later, a DSB was induced by transfection with IScel expression vector. Cells were fixed with 1% formaldehyde 16 h later and then washed with ice-cold 0.5% BSA/PBS. Fixed cells were resuspended in lysis buffer (1% SDS, 10 mM EDTA, 0.5 mM EGTA, 50 mM Tris-HCl pH 7.5, 0.2% Triton X-100, protease and phosphatase inhibitor cocktail), and sonicated to achieve a desired median fragment length of 200 bases. Lysates were then diluted in IP buffer (1% Triton X-100, 2 mM EDTA, 150 mM NaCl, 20 mM Tris-HCl pH 7.5, protease and phosphatase inhibitor cocktail). 5% of the solution was reserved as input. The remaining pre-cleared lysates were used for overnight immunoprecipitation with either BRCA1 antibody (A300-000, Bethyl Labs) or IgG control (ab37415, Abcam) and pre-washed beads. The following day, beads were washed with SDS-free RIPA/LiCl buffer (50 mM HEPES pH 7.5, 1 mM EDTA, 0.7% Sodium deoxycholate, 1% NP-40, 0.5 M LiCl, protease and phosphatase inhibitor cocktail) followed by washes in 1× TE buffer. Immunoprecipitated beads were resuspended in elution buffer (1% SDS, 0.1 M Sodium bicarbonate) and incubated at 65° C. to reverse the crosslinks. Eluates were further subjected to proteinase K treatment for 2 hours at 37° C. and purified using Qiagen. 820 qPCRs were conducted according the protocol below along with the following primers:

TABLE 9

| Target | | |
|---|---|---|
| Gene | Forward Primer | Reverse Primer |
| DR-1 | 5′-GAGCAAGGGCGAGGAGCTGT-3′ | 5′-CCGTAGGTCAGGGTGGTCAC- |
| DR-2 | 5′- | 5′- |
| DR-3 | 5′-TGCTGCCCGACAACCACTAC-3′ | 5′-GTACAGCTCGTCCATGCCGA-3′ |
| DR-4 | 5′-GAGGGCGAGGGCGATGCC-3′ | 5′-GCCTTCGGGCATGGCGGA-3′ |
| GAPDH | 5′-GCTTGCCCTGTCCAGTTAAT-3′ | 5′-TAGCTCAGCTGCACCCTTTA-3′ |

Cell Cycle Analysis. Unsynchronized DR-GFP U2OS were transfected with either siRNA control and siRNA against EWSR1 by reverse transfection or empty vector and EWS-FLI1 by forward transfection 24 hours after seeding. 48 hours post-transfection, cells were pulse-labeled with 10 mM BrdU for 30 min and fixed with 70% ice-cold ethanol for 30 min on ice. After centrifugation, the cell pellet was treated with RNase A and resuspended in PBS containing 50 μg/ml Propidium Iodide (PI). DNA content was analyzed on a BD flow cytometer and FlowJo software. Similar procedure was conducted for TC32 cells transfected with either Empty Vector or RNASEH expression constructs.

RNASEH Activity assay. Enzyme activity was assessed using a FRET-based assay as previously described. 10 μM fluorescein-labeled oligonucleotides were annealed with complementary 5′-dabcyl-labeled oligonucleotides in 60 mM KCl, 50 mM Tris-HCl pH 8.0. Assays were conducted in 100 μl volume in 96 well plate format. Each reaction consisted of 60 mM KCl, 50 mM Tris-HCl (pH 8.0), 10 mM MgCl₂, 0.25 μM oligonucleotide duplex and 10 μl whole cell lysates. 2.5 units of E. coli RNase H (NEB) was used as positive control. The assay plate was incubated at 37° C. for 3 hours with intermittent shaking and fluorescence was read at 480 nM excitation and a 535 nM emission filter. Fluorometric substrates: Incorporated RNA substrate (GATCTGAGCCTGGGaGCT upper strand, AGCTCCCACiGCTCAGATC lower strand; uppercase denotes DNA; lowercase. RNA), DNA:RNA substrate (gatctgagcctgggagct upper strand, DNA complementary lower strand), ds RNA substrate (gatctgagcctgggagct upper strand, RNA complementary lower strand), dsDNA substrate (GATCTGAGCCTGGGAGCT upper strand, DNA complementary lower strand).

Quantitative PCR. Real-time quantitative PCR (qPCR) was performed on DNA samples obtained either after chromatin immunoprecipitation or DNA:RNA immunoprecipitation using SYBR qPCR Mix (Applied Biosystems) according manufacturer's protocol. The following primers were used for qPCR:

TABLE 10

| Target | | |
|---|---|---|
| Gene | Forward Primer | Reverse Primer |
| APOE | 5′-CCGGTGAGAAGCGCAGTCGG-3′ | 5′-CCCAAGCCCGACCCCGAGTA-3′ |
| EGR1 | 5′-TTCGGATTCCCGCAGTGT-3′ | 5′-TCACTTTCCCCCCTTTATCCA-3′ |
| SNRPN | 5′-TGCCAGGAAGCCAAATGAGT-3′ | 5′-TCCCTCTTGGCAACATCCA-3′ |
| PARP8 | 5′-GGGTGTCCTTAGGCAGAACA-3′ | 5′-ATGGAAACCTGTTTGGCTTG-3′ |
| FEN1 | 5′-CCTCTCGCCCTTAGAAATCG-3′ | 5′-TAGACGCTCCTGGAACCTC-3′ |

Statistical Analysis. P-value for comparing cell viability, R-loop intensity, fold change in repair assays and enzyme assays were derived using Student's t test (two-tailed, GraphPad Prism 6) or non-parametric Mann-Whitney rank sum t-test, unless otherwise specified. When performing multiple comparisons, an FDR of 1% was used as cutoff as evaluated by Benjamini-Hochberg method. P-value<0.01 or 0.001 was considered significant changes (marked as *, #, or **, ## respectively).

RNAi screens. The RNAi screens with etoposide, bleomycin and MMS were performed as described previously. 1.2×10⁴ Kc67 cells (Drosophila melanogaster) were seeded in 384-well plates with Schneider medium and grown at 22° C. in a humidified chamber. Each well of a 384-well plate contained 0.25 μg of double-stranded RNA (dsRNA) with 22,915 dsRNA representing the whole library. The top 5% “survival” hits for each damaging agent was calculated (Table 6) by a previously published method.

RNA-seq and gene expression analysis. IMR90, U20S, TC32, EWS502, CHLA258 and CHLA10 cell lines were grown in 10 cm dishes to 90% confluence. Samples were collected after 0, 6, 12, 18, 24 hours of etoposide exposure (equitoxic doses leading to 65% viability after 72 hours) and RNA extracted using Qiagen RNeasy kit. The quality of RNA samples was analyzed by Agilent 2100 BioAnalyzer. Sequencing libraries were prepared from total RNAs according to Illumina's RNA sample preparation protocol. Samples were barcoded, and pooled and sequenced with a HiSeq 2000 system with the 50 bp paired-end protocol, and with targeted read counts around 30 million reads. Only U2OS samples were sequenced with 50 bp single-end protocol. We used TopHat2 aligner to map paired reads to UCSC hg19 genome build. For gene expression level quantification, we used HTSeq to obtain raw read counts per gene and then converted to RPKM (Read Per Kilobase of gene length per Million reads of the library) according to gene length and total mapped read count per sample. Log2-transfromed RPKM measurement was used as gene expression level. Differential expression analysis and functional annotation classification was conducted using Gene Set Enrichment Analysis (Broad Institute) and DAVID. Publicly available gene expression datasets were mined from the R2 database.

Chromatin Immunoprecipitation (ChIP). Cells were grown to ˜80% confluence and treated with etoposide (LD65) for six hours. Chromatin was harvested after fixing with 1% formaldehyde and sheared to average length of 200-1500 bp using a Branson sonicator. 400 μg of sheared chromatin was added to 50 μl of Protein G beads that were pre-incubated with 10 μg of antibody—RNAPII (GAH-111, Qiagen), BRCA1 (sc-646, Santa Cruz) and IgG (GAH-111, Qiagen or ab37415, Abcam), for overnight incubation. ChIP DNA was eluted by incubating beads in elution buffer (50 mM Tris pH 8.0, 10 mM EDTA and 1% SDS) overnight at 65° C., followed by sequential treatments with RNaseA and Proteinase K. The final ChIP DNA was purified by phenol/chloroform method and ethanol precipitation. These samples were further sheared using Covaris sonicator to get an average length of 350 bp. Library construction and purification was done following manufacturer's protocol (MicroPlex Library Preparation kit, Diagenode and AgenCourt Ampure XP, Beckman Coulter). Two control and two EwS cell lines were used for sequencing.

DNA:RNA Immunoprecipitation (DRIP). DRIP was performed according to previously published protocol. Briefly, DNA from a nearly confluent 10 cm dish was obtained using Proteinase K followed by phenol/chloroform extraction and ethanol precipitation. DNA was subjected to overnight digestion using a cocktail of restriction enzymes (HindIII, EcoRI, BsrGI, XbaI and SspI, NEB). Post-cleanup with phenol/chloroform/ethanol, 4 μg of digested DNA with or without RNaseH pre-treatment was used as input for immunoprecipitation using S9.6 antibody (Kerafast). DNA:Antibody complex was incubated for 16 hours and allowed to bind protein A/G beads for a further 4 hours. Bound DNA fragments were recovered in the elution buffer by incubating with Proteinase K at 55° C. for 45 min. Recovered DNA was cleaned using phenol/chloroform/ethanol and resuspended in 10 mM Tris-HCl pH 8.0. Each IP was run in triplicate and samples were pooled for sequencing after sonication.

ChIP and DRIP Sequencing and Peak Identification. Sonicated and size selected DNAs (immunoprecipitated DNAs that was not treated, etoposide treated or RNase H treated, and input DNA) were processed according to Illumina Genome DNA library preparation protocol, and sequenced with a HiSeq 2000 or a HiSeq 3000 system with 50 bp single-read sequencing protocol. On average, 30-40 million reads were generated for each DNA sample, and then aligned to UCSC hg19 genome build by using BWA. Peak calling was performed by using MACS2 algorithm. Similar to Ginno, P A et al., we use peak calling parameter with 5-fold up to 30-fold enrichment over corresponding input DNA as control (MACS2 parameter: -g 2.7e9 -q 0.05 -B -m 5 30).

Determination of consensus ChIP and DRIP regions. The block diagram of the algorithm for determination of consensus DRIP regions is shown in FIG. 22A. The methodology for analysis has been described in detail in Liu R, et al. Briefly, DRIP regions were first stacked according to their genomic position (within each chromosome), and then regions that were present in at least 3 DRIP samples (under any condition) were selected. If the next adjacent region is less than 200 bp away, we treated them as being contiguous. Regions smaller than 200 bp were eliminated. With each seed region, we extended it to the longest run within the stack, followed by another merging step between adjacent regions that are less than 200 bp apart. A total of 33,121 DRIP regions were detected. SmallDRIP regions (FIG. 23) were determined by removing samples with strong DRIP peaks (EWS502, CHLA10, and TC32), and then performing the consensus peak detection algorithm again.

ChIP and DRIP region coverage. We quantified DRIP regions by its coverage and sequence read count. Clearly, DRIP region with greater coverage and higher enrichment (large sequence read count), were indicative of a true DRIP peak (FIG. 22A and FIG. 24). After obtaining the consensus regions, coverage of each sample was defined as: In other words, the coverage of a given sample is the percent of consensus peaks that are covered by the original DRIP peaks obtained by the MACS algorithm.

Coverage=Σ_{all peaks} Overlap of detected DRIP peaks with Consensus regions (bp)/Σ_{all Consensus Peaks} Consensus Regions (bp)

Normalized read counts. To quantify the read counts within the DRIP regions, we first counted short sequence reads within 100 bp tiling bins equally across entire genome using BedTools/CoverageBed command. And then the normalized read count of a given DRIP region was quantified as:

$$ReadCount = \frac{\begin{matrix}( 1 / N ) {\sum }_{all bins overlap with consensus DRIP regions} \\ reads in each bin * 50 / 100\end{matrix}}{Total number of mapped reads / 10 , 000 , 000}$$

where N is the total number of DRIP peaks. ReadCount per DRIP region was normalized to 10 million reads per library. By considering read length (50 bp) and bin size (100 bp), the ReadCount unit essentially defines the normalized depth of coverage per 10 million reads per library. Therefore, if we use consensus DRIP regions for all samples, ReadCount of a given DRIP region becomes comparable between samples.

ENCODE ChIP-seq Data. BRCA1 ChIP-seq data was obtained from ENCODE project website (https://www.encodeproject.org/, ENCSR000EDB and ENCSR000EBX). Data was downloaded in order of priority listed below: (1) BAM files were converted to read count by using CoverageBed command in BedTools. (2) UniformlyProcessedPeakCalls files were used as is to indicate corresponding binding peak sites. If not, we used narrowPeaks.peak annotation were performed using Cis-regulatory Element Annotation System(CEAS) package (v1.0.2). (3) If peak calling file was not available, we used BAM file and it's corresponding input BAM file to generate peak calls by using MACS2 algorithm, followed by CEAS for annotation. (4) If BAM file does not exist, we downloaded fastq files, utilized BWA aligner to generate a BAM file, and then followed step 3 to obtain ChIP binding sites.

Gene sets derived from EWS ChIP-seq or other profiling techniques. EWS-FLI1 targeted genes determined by ChIP-seq were extracted from from Riggi et al. 1785 EWS-FLI1 binding sites were reported, which we mapped to 1314 unique genes (Table 11).

TABLE 11
Breakdown of EWS-FLI1 binding sites based on sequence characteristics.
The set of 1785 EWS-FLI1 binding sites obtained from Riggi
et al. was classified according to type of sequence - canonical
ETS binding site (Single GGAA), novel repetitive binding
site (Consecutive GGAA) or other.

| | Consecutive | Single | | |
|---|---|---|---|---|
| EWS-FLI1 Binding sites | GGAA | GGAA | Other | Total |
| Number of Binding sites reported | 1011 | 330 | 444 | 1785 |
| Number of unique genes mapping | 768 | 309 | 424 | 1314 |
| to the binding sites | | | | |
| Number of genes shared with | | 72 | 101 | |
| Consecutive GGAA | | | | |

Two types of sequences were discussed—the canonical GGAA motif (Single GGAA) that is recognized by all ETS DNA binding domain proteins and the novel repeat GGAA sequences motif (Consecutive GGAA) that were bound exclusively by EWS-FLI1. As our BRCA1-ChIP data indicated an overwhelming majority of BRCA1 binding sites were in the transcription start site (TSS) we further divided these categories into binding sites that were within 10 kb or outside 10 kb of the TSS (Table 12).

Table 12. Breakdown of EWS-FLI1 binding sites as a function of distance to transcription start site. The sub-groups (Single GGAA, Consecutive GGAA and Other) were used for this analysis and the number of unique genes that correspond to these loci is also presented.

TABLE 12

| Distance of EWS-FLI1 | | Unique | Consecutive | Single | |
|---|---|---|---|---|---|
| binding sites | Genes | genes | GGAA | GGAA | Other |
| Within 10 kb of TSS | 284 | 280 | 100 | 74 | 139 |
| Greater than 10 kb | 1400 | 1012 | 652 | 231 | 280 |
| from the TSS | | | | | |

DRIP-seq and ChIP-seq heatmap. For any given gene set, a heatmap of DRIP data, ChIP data and gene expression data was generated for correlation examination. Gene set was provided in BED format that contains at least 5 columns: 1) chromosome; 2) genomic start position, 3) genomic end position, 4) Unique ID, 5) Gene Symbol. All genomic data are derived from UCSC hg19 genome build. If TSS, the Columns 2 and 3 will be +/−100 bp around TSS defined by the farthest transcription start sites, when multiple transcripts are defined in UCSC hg19 “refFlat” table. If the genes were derived from ChIP-seq data, then the genomic positions are simply the DNA binding sites from a given pull-down target. Heatmap selects 50 bins (5,000 bp) to the left and right (x-axis) for a given gene set (y-axis), then plot the ReadCount value from white (read count 0) to dark read. Gene set (y-axis) may be sorted by the peak value defined in following sections. DRIP-seq: raw sequence read counts around +/−5,000 bp (50 bins to each side, with bin of 100 bp) were extracted around the center of each site defined in the gene set. Each gene was also represented by mean read count over 100 bins. If a specific order for DRIP data was requested, it was sorted again by read counts. To generate color map, we use a color scheme as follows: white—no reads, magenta—with detectable read counts, and black/dark magenta—height peaks. DRIP-seq data was normalized to 20 million reads per library equivalent.

ChIP-seq: similar to DRIP-seq data, raw read counts +/−5,000 bp were extracted around the center of each site defined in the gene set. Given that BRCA1 binding sites are mostly narrow peaks, we calculated the mean peak height over 7 consecutive bins (700 bp) windows, and then took maximum height over all moving 7 bin windows, or

$$peak height = {max}_{j = 3 to 47} ( \frac{1}{7} {\sum }_{k = - 3}^{3} {RC}_{j + k} )$$

where RC is the read count of a given bin. By doing so, we allow the ChIP-seq peak to be within the region, but not necessary situated at the center, particularly when TSSs or TTSs were requested. Heatmap was generated using the same color scheme as DRIP-seq. Code for rendering the heatmaps was written in Matlab and will be made available upon request.

Gene-expression: Since gene expression data were extracted by using the gene symbol matching. Log2-transfromed RPKM was used to represent expression level. Heatmap color scheme is as follows: magenta—higher expression, and blue—lower expression.

Kolmogorov-Smirnov (KS) Test for enrichment. A flow diagram detailing the steps of the KS test algorithm is presented in FIG. 22B and FIG. 23. For a given gene list, we sorted ChIP data, gene expression data, DRIP, RNAPII and BRCA1-ChIP data (as shown in FIG. 27B, FIGS. 28A and 28B, FIG. 41B, FIGS. 42A and 42C, and FIG. 43B) according to either the ChIP-seq peak height, gene expression level, or DRIP-seq mean read count. To demonstrate the concordance of ChIP peaks and DRIP peaks, for example, we hypothesized that if concordance does not exist, then the DRIP peaks will be uniformly distributed within the gene list. Therefore, we first determined the ChIP peak threshold (Peak height>9), DRIP peak threshold (ReadCount>m+MAD*1.89) and expression>5. Depending on the number of peaks we got from ChIP-seq (for example), we determined the threshold for expression and DRIP-seq such that both will have the same positive number of peaks/genes, and we then examined there location within gene list and examine whether the are uniformly distributed using KS test. Code for performing the statistical test was written in MATLAB and will be made available upon request.

Further modifications and alternative embodiments of various aspects of the compositions and methods disclosed here will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described here are to be taken as examples of embodiments. Elements and materials may be substituted for those illustrated and described here, parts and processes may be reversed or omitted, and certain features of the embodiments may be utilized independently, all as would be apparent to one skilled in the art after having the benefit of this description of the embodiments. Changes may be made in the elements described here without departing from the spirit and scope of the embodiments as described in the following claims.

The foregoing descriptions of methods, compositions, and results obtained using them are provided merely as illustrative examples. Descriptions of the methods are not intended to require or imply that the steps of the various embodiments must be performed in the order presented. As will be appreciated by one of ordinary skill in the art, the steps in the foregoing embodiments may be performed in any order. Words such as “then” are not intended to limit the order of the steps; these words are simply used to guide the reader through the description of the methods. Many of the operations may be performed in parallel or concurrently. In addition, the order of the operations may be re-arranged. A process may correspond to a method, a function, a procedure, a subprogram, etc. Various modifications to these embodiments will be readily apparent to those skilled in the art, and the generic principles defined here may be applied to other embodiments without departing from the spirit or scope of the disclosure.

What is claimed is:

1. (canceled)

2. A method for diagnosing and treating cancer in a subject comprising:
analyzing a tissue sample from the subject for the presence or absence of a plurality of R-loops, wherein the subject is diagnosed with the cancer if the plurality of R-loops is detected; and
administering the subject with a therapeutically effective amount of an inhibitor of Ataxia Telangiectasia and Rad3 related Serine/Threonine protein kinase and an inhibitor of poly (ADP-ribose) polymerase 1 or pharmaceutical acceptable salts thereof to the diagnosed subject.

3. A method for diagnosing and treating cancer in a subject comprising:
analyzing a tissue sample from the subject for phosphorylation levels of RNA polymerase II, wherein the subject is diagnosed with the cancer if an increase in phosphorylation levels of RNA polymerase II is detected; and
administering the subject with a therapeutically effective amount of an inhibitor of Ataxia Telangiectasia and Rad3 related Serine/Threonine protein kinase and an inhibitor of poly (ADP-ribose) polymerase 1 or pharmaceutical acceptable salts thereof to the diagnosed subject.

4. (canceled)

5. A method for diagnosing and treating cancer in a subject comprising:
analyzing a tissue sample from the subject for the presence or absence of a plurality of R-loops, wherein the subject is diagnosed with the cancer if the plurality of R-loops is detected; and
administering the subject with a therapeutically effective amount of an inhibitor of Checkpoint kinase 1 and an inhibitor of poly (ADP-ribose) polymerase 1 or pharmaceutical acceptable salts thereof to the diagnosed subject.

6. (canceled)
